(19) United States
(12) Patent Application Publication
(10) Pub. No.: US 2024/0423255 A1
(43) Pub. Date: Dec. 26, 2024

(54) METHODS FOR MANUFACTURING SMALL MOLECULE BIOACTIVES

(71) Applicant: Brightseed, Inc., South San Francisco, CA (US)

(72) Inventors: Gul Konuklar, Alameda, CA (US); Armando Alfonseca, American Canyon, CA (US); Sophia Pinton, San Francisco, CA (US)

(21) Appl. No.: 18/826,672

(22) Filed: Sep. 6, 2024

Related U.S. Application Data
(63) Continuation of application No. PCT/US2023/063874, filed on Mar. 7, 2023.
(60) Provisional application No. 63/420,231, filed on Oct. 28, 2022.
(60) Provisional application No. 63/317,708, filed on Mar. 8, 2022.

(51) Int. Cl. A23L 25/00; A21D 2/02; A21D 2/18; A21D 2/26; A21D 2/36; A23C 9/152; A23C 9/154; A23C 9/156; A23L 29/00; A23L 33/21; A23P 30/20; A23P 30/30; A61K 31/00; A61K 31/165

(52) U.S. Cl. CPC A23L 25/30; A21D 2/02; A21D 2/181; A21D 2/266; A21D 2/36; A23C 9/1522; A23C 9/1526; A23C 9/1544; A23C 9/156; A23L 29/015; A23L 33/21; A23P 30/20; A23P 30/30; A61K 31/165; A61K 31/658; A61K 36/3482

(57) ABSTRACT

Aspects of the disclosure relate, inter alia, to purer extracts of a bioactive compound from minimal processing. In general, when developing ingredients that comprise small molecule bioactive compounds, the processing traditionally may require combined consecutive multiple process unit operations that in turn might require high shear and high pressure. Aspects of the disclosure relate to processing food ingredients with minimal distress imposed on the bioactive compounds.

### CROSS-REFERENCE TO RELATED APPLICATIONS

This application is a continuation of U.S. National Phase of International No. PCT/US2023/063874, filed Mar. 7, 2023, and claims the benefit of U.S. Provisional Application Nos. 63/420,231, filed Oct. 28, 2022 and 63/317,708, filed Mar. 8, 2022, which are incorporated by reference in their entirety.

### FIELD OF THE DISCLOSURE

The present disclosure relates to food products and their methods of preparation. More particularly, the present disclosure relates to whole food ingredients and extracts with one or more small molecule bioactives and their methods of manufacture and preparation.

### BACKGROUND

Various natural products are produced, processed, and used in large quantities and in a number of applications. Often such materials are used once or discarded as waste, or are simply considered to be waste material. However, these materials can be can transformed into exceptional nutritional or pharmaceuticals. Yet, there remains a need for ways of producing whole food ingredients or extracts that retain bioactives.

### SUMMARY OF THE DISCLOSURE

Aspects of the disclosure relate to an integrated extruder. In some embodiments, the integrated extruder includes a twin screw barrel assembly, and two co-rotating screws housed within the twin screw barrel assembly. In some embodiments, the twin screw barrel assembly further comprises one or more feeders, compounders, mixers, reactors, or a combination thereof. In some embodiments, wherein the twin screw barrel assembly further comprises a heater or cooling system. In some embodiments, the twin screw barrel assembly further comprises a mesh filter. In some embodiments, the twin crew barrel assembly comprises a plurality of barrels which are connected to each other. In some embodiments, the plurality of barrels may comprise a combi barrel with a hole in the side allowing for an additional input or an additional output. In some embodiments, the twin screw barrel assembly comprises a first feeder positioned adjacent to a barrel configured to allow material to be input into the twin barrel assembly. In some embodiments, the two screw barrel assembly comprises a second feeder positioned adjacent to a combi barrel configured to allow for the input of feed material into the twin barrel assembly. In some embodiments, the integrated extruder further includes a die plate positioned at the end of the barrel assembly. In some embodiments, the integrated extruder further includes a pressure release opening valve attached to the twin screw barrel assembly. In some embodiments, the integrated extruder further includes a water injection element attached to the twin screw barrel assembly.

Aspects of the disclosure relate to a process for preparing a food composition. In some embodiments, the method includes roasting hemp hulls to form roasted hemp hulls, milling roasted hemp hulls to form milled roasted hemp hulls, and feeding milled roasted hemp hulls into an integrated extruder as described herein to form a hemp hull extract ingredient. In some embodiments, the process further includes pre-treating the milled roasted hemp hulls with water, enzymes, acid, base, or a combination there. In some embodiments, the process further includes high shear mixing the milled roasted hemp hulls. In some embodiments, the method further includes applying a high pressure extraction to the milled roasted hemp hulls. In some embodiments, the process further includes separating one or more solids and hemp hull extracts from the milled roasted hemp hulls. In some embodiments, the process further includes roasting hemp hulls is at a temperature range from about 225° C. to about 275° C. In some embodiments, the milling roast hemp hulls is at speed from about 150 rpm to about 250 rpm.

Aspects of the disclosure relate to a process for preparing a food composition. In some embodiments, the process includes roasting hemp hulls to form roasted hemp hulls, milling roasted hemp hulls to form milled roasted hemp hulls, and feeding milled roasted hemp hull into an integrated extruder as described herein to form a hemp hull whole food ingredient or hemp hull puff or crisp. In some embodiments, the process further includes pre-treating the milled roasted hemp hulls with water, enzymes, acid, base, or a combination there. In some embodiments, the process further includes high shear mixing the milled roasted hemp hulls. In some embodiments, the process further includes puffing, pelletizing or shaping the milled roasted hemp hulls. In some embodiments, the process further includes vacuum drying the milled roasted hemp hulls. In some embodiments, the process further includes granulating the milled roasted hemp hulls. In some embodiments, roasting hemp hulls is at a temperature range from about 225° C. to about 275° C. In some embodiments, the milling roast hemp hulls is at speed from about 150 rpm to about 250 rpm.

Aspects of the disclosure relate to a food composition. In some embodiments, the food composition includes extruded hemp hulls, wherein the extruded hemp hulls retain one or more bioactives from an extrusion process as described herein. In some embodiments, the food composition is a liquid product. In some embodiments, the food composition is an extract ingredient. In some embodiments, the food composition is a whole food ingredient. In some embodiments, the food composition is a pressed cake. In some embodiments, the food composition is a cracker. In some embodiments, the food composition is a cereal. In some embodiments, the food composition is an extruded puff. In some embodiments, the one or more bioactives is a tyramine containing hydroxycinnamic acid amide. In some embodiments, the tyramine containing hydroxycinnamic acid amide is selected from the group consisting of N-trans-caffeoyltyramine, N-cis-caffeoyltyramine, N-trans-feruloyltyramine, N-cis-feruloyltyramine, p-coumaroyltyramine. In some embodiments, the food composition further includes one or more micronutrients. In some embodiments, the food composition further includes one or more acceptable excipient or carrier for oral consumption.

Aspects of the disclosure relate to a method for making a liquid product. In some embodiments, the method includes adding hemp hulls to an extruder as described herein at a speed from about 500 rpm to about 700 rpm, wherein the hemp hulls are added at a rate from about 3 lb/h to about 8 lb/h, wherein water is added from about 5 lb/h to about 10 lb/h, wherein the temperature is from about 130° C. to about 150° C., and screening the hemp hulls with a mesh screen, thereby collecting the liquid product.

Aspects of the disclosure relate to a method for making an expanded food product. In some embodiments, the method includes forming a dough by admixing water and hemp hulls, extrusion cooking the dough to form a cooked dough, extruding the cooked dough through an extruder as described herein to form cooked dough pieces, and cutting the cooked dough pieces, wherein the cooked dough pieces retain one or more bioactives.

Aspects of the disclosure relate to a method of producing a hemp hull containing composite food product. In some embodiments, the method includes extruding hemp hulls with an extruder as described herein, thereby forming a milled hemp hull, and adding the milled hemp hull to addition food ingredients, thereby producing a hemp hull containing composite food product.

### BRIEF DESCRIPTION OF THE DRAWINGS

The features and advantages of the compositions and methods described herein will become apparent from the following description, taken in conjunction with the accompanying drawings. These drawings depict certain aspects of the compositions and methods described in the present application, and thus, are not to be considered limiting. In the drawings, similar reference numbers or symbols typically identify similar components, unless context dictates otherwise. The drawings may not be drawn to scale.

FIG. 1 shows a perspective view of the twin screw extruder in accordance with an aspect of the disclosure.

FIG. 2 shows a perspective view of an extruder in accordance with a second aspect of the disclosure.

FIG. 3 shows a perspective view of twin screws of an aspect of the disclosure.

FIG. 4 illustrates an embodiment of a process and product pathways for whole food ingredient or extract ingredient.

FIG. 5 illustrates a flow chart of an embodiment of the disclosure to produce a hemp hull extract powder.

FIG. 6 illustrates a flow chart of an embodiment of the disclosure to produce hemp hull whole food ingredient powder or hemp hull puff/crisp.

FIG. 7 illustrates a flow chart of an embodiment of the disclosure to produce black pepper whole food ingredient powder or black pepper puff/crisp.

FIG. 8 illustrates a bar graph depicting bioactive production from an extruder in accordance with an aspect of the disclosure.

FIG. 9 illustrates a bar graph depicting bioactive production from an extruder in accordance with an aspect of the disclosure.

FIG. 10 illustrates a photo of a puffed crisp product in accordance with an aspect of the disclosure.

FIG. 11 illustrates images of cereal products in accordance with an aspect of the disclosure.

FIG. 12 illustrates a photo of a puffed product in accordance with an aspect of the disclosure.

FIG. 13 illustrates a photo of a pressed cake product in accordance with an aspect of the disclosure.

FIG. 14 illustrates a photo of liquid products in accordance with an aspect of the disclosure.

FIG. 15 illustrates a photo of a whole food ingredient powder in accordance with an aspect of the disclosure.

FIG. 16 illustrates a photo of a whole food ingredient solid in accordance with an aspect of the disclosure.

FIG. 17 illustrates a photo of a whole food ingredient product in accordance with an aspect of the disclosure.

FIG. 18 illustrates a photo of a roasted whole food ingredient product in accordance with an aspect of the disclosure.

FIG. 19 illustrates a table describing the microbiological results of inoculated Enterococcus surrogated in Hemp Hull Fiber process extruder trial.

FIG. 20 illustrates a table describing the microbiological results of inoculated Enterococcus surrogated in Hemp Hull Fiber process extruder trial.

### DETAILED DESCRIPTION OF THE DISCLOSURE

Whole food ingredients may include beneficial bioactive compounds, and are beneficially added to food products after minimal processing. Aspects of the disclosure relate, inter alia, to purer extracts of the bioactive compound from minimal processing. In general, when developing ingredients that comprise small molecule bioactive compounds, the processing traditionally may require combined consecutive multiple process unit operations that in turn might require high shear and high pressure. Aspects of the disclosure relate to processing food ingredients with minimal distress imposed on the bioactive compounds within the feedstock. Aspects of the disclosure relate to extruders that, unlike some other equipment, may be advantageously designed to accommodate many unit operations in a continuous fashion, rather than individual batch processes, and still achieve high shear and high-pressure levels while still being gentle to the materials. Aspects of the disclosure relate to processing that maintains the natural state of these bioactive compounds. Aspects of the disclosure relates to processing with very short processing times. Aspects of the disclosure relate to methods of processing whole food ingredients while simultaneously (or in the alternative) preserving rich fiber content and bioactive ingredients within the whole food ingredients.

Aspects of the disclosure relate, inter alia, to small molecule bioactives, extrudable bioactive compositions, methods for manufacturing a bioactive product, and methods for delivering the bioactive contained in the product to a user. Bioactives, as described herein, may be small molecule compounds produced by plants, fungi, and microbes that grow in and adapt to environments that present a wide range of challenges to their survival. Bioactives also benefit the health of humans—for example, the caffeine in tea, the powerful antioxidant lycopene in tomatoes, and the potent anti-inflammatory curcumin. Bioactives also may inform active ingredients in medicines like Taxol, Aspirin, and Metformin—a first-line treatment for Type 2 diabetes, derived from the French lilac flower.

Aspects of the disclosure relate, inter alia, to a food product. In some embodiments, the food product contains a whole food ingredient. A whole food ingredient may comprise one or more bioactive products. Aspects of the disclosure relate to methods for manufacturing a food product. A food product may be a finished product ready for the consumer to eat directly and may also be a component, such as a powder, that a consumer or manufacturer may add to other foods before eating. In addition to the bioactive benefits on gut strength, whole food ingredients, according to the disclosure, may offer a high concentration of dietary fiber content and a facile method for manufacturers to easily achieve an excellent source of fiber content in their products by incorporating whole food ingredients according to methods described herein.

Aspects of the disclosure are directed to a food product comprising extrudable bioactive compositions. Food products of this disclosure may include all types of puffed products, including, for example, puffed crisps. Food products of this disclosure may also include crisps, cereals, bars, whole food ingredient powders, and bars. Advantageously, some food products of the disclosure comprising a whole food ingredient may have superior food attributes. For example, according to aspects of the disclosure, puffed products may have improved crunchiness compared to the same puffed product without a whole food ingredient. In some embodiments, puffed products, according to aspects of the disclosure, may have improved structural properties such that the puffed product is resistant to collapsing or deflating. In some embodiments, puffed products, according to aspects of the disclosure, may have improved structural properties, including a strong shell structure. In some embodiments, a strong shell structure may improve the puffed product's texture, structural integrity, and or longevity. In some embodiments, puffed products, according to aspects of the disclosure, may be resistant to crumbling, as compared to, for example, the same puffed product without a whole food ingredient.

Food products of this disclosure may advantageously be mixed with other ingredients that lack high structural integrity. In some embodiments, food products of this disclosure may be mixed with ingredients like pea protein, which may otherwise, on its own, create a weak structure. In some embodiments, food products of this disclosure may be mixed with ingredients containing amylose containing starches, which may otherwise, on its own, create a weak structure. Advantageously, whole food ingredients may improve the puffed products' drying process by either the rate of drying or the total amount of drying.

Unexpectedly, food products of this disclosure are observed to tolerate both high levels of a whole food ingredient and high levels of protein while retaining structural integrity. While a manufacturer may typically vary the concentrations of various ingredients in a food product, food products of this disclosure were observed to tolerate surprising levels of protein, including at levels outside of what was known in the art. Also, unexpectedly, even a high amount of a whole food ingredient produced good puffed products that did not have inhibited puffing. Additionally, whole food ingredients, according to this disclosure, were observed to be easily combined with most protein combinations, even with soy protein. Unexpectedly, a high amount of a whole food ingredient may be helping the puffing action (enhancing). In some embodiments, a food product may be a puffed product with a high amount of a whole food ingredient.

Advantageously, some food products of the disclosure comprising a whole food ingredient may have additional superior food attributes. Unexpectedly, puffed products, according to this disclosure, were observed to have enhanced melt characteristics in the mouth (saliva). In some embodiments, the food products, according to this disclosure, have delayed response to moisture uptakes when soaked in liquids (including, for example, milk, artificial milks, water, and yogurts). Accordingly, in some embodiments, the food products, according to this disclosure, stays crispier before and or during consumption. Puffed products according to this disclosure were also observed to decrease or cover some off notes and sensory defects coming from other ingredients. In some embodiments, the food product according to this disclosure ameliorated the sensory defects from ingredients including pea protein and soy protein.

Aspects of this disclosure are also directed to parameters for an optimal crisp formulation comprising a whole food ingredient and optimal methods for crisp formulation.

### Definitions

Additionally, the phrase “consisting essentially of” will be understood to include those elements specifically recited and those additional elements that do not materially affect the basic and novel characteristics of the claimed technology. The phrase “consisting of” excludes any element not specified. The transitional phrase “consisting of” excludes any element, step, or ingredient not specified in the claim.

Where a range of values is provided, it is understood that the upper and lower limit, and each intervening value between the upper and lower limit of the range is encompassed within the embodiments.

As used herein, and unless otherwise indicated, percentage (%) refers to total % by weight typically on a dry weight basis unless otherwise indicated.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value. Where particular values are described in the application and claims, unless otherwise stated the term “about” meaning within an acceptable error range for the particular value should be assumed.

As used herein, the term “antioxidants” refer to compounds that interrupt the free radical reaction chain.

As used herein, the term “agglomeration” refers to a process of transforming fine particles into larger particles by the addition of an agglomerating agent and introduction of an external force.

As used herein, the term “bioactive substances” broadly include any compound, or mixtures thereof, that can be delivered by the microparticles to produce a beneficial result in an organism to which the compound or mixture has been delivered. The bioactive substances can be living or dead microbes, bacteriophages and viruses, soluble or limited solubility compounds, such as an oil phase, powder, or other solid form.

As used herein, a “carrier” or “excipient” as used herein are broad terms, and each is to be given its ordinary and customary meaning to a person of ordinary skill in the art (and is not to be limited to a special or customized meaning), and refers without limitation to a compound that facilitates the incorporation of a compound into cells or tissues.

As used herein, the term “emulsifiers” refers to a molecule made up of a lipophilic component and a hydrophilic component. Typical examples of emulsifiers are lecithins, monoglycerides, diglycerides, phosphated monoglycerides, esters of monoglycerides, succinic acid esters of monoglycerides, diacetyl tartaric acid esters of monoglycerides, sucrose esters of fatty acids, propylene glycol esters of fatty acids, sorbitan esters of fatty acids, polyglycerol esters of fatty acids.

As used herein, “fibrous” refers to containing, consisting of, or resembling fibers, threads, filaments, or groups of filaments grouped into a continuous strand; wherein the strands can be easily separated.

As used herein, the term “high-shear mixer” refers to an apparatus that disperses or transports one phase or ingredient (liquid, solid, or gas) into a main continuous phase (liquid), with which it would normally be immiscible.

As used herein, the term “course screen” refers to a screen or mesh to separate solids, which can be used to produce solid materials, from the liquid pasteurized hydrolysate, and can include a variety of screening techniques.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration.

As used herein, the term “disintegrant” includes conventional disintegrants and other disintegrants known in the art as super-disintegrants.

As used herein, the term “extrudate” or “extruded product” refers to a product comprising cultivated animal cells, plant protein, at least one other ingredient, and optionally a peptide cross-linking enzyme, the extrudate is prepared by use of an extrusion machine. The extrudate can optionally comprise a peptide cross-linking enzyme.

As used herein, the term “fiber” is carbohydrate polymer obtained from plants that cannot be completely broken down by the human digestive system when consumed. Soluble fiber is soluble in water and insoluble fiber does not dissolve in water.

As used herein, the term “fibrous” or “fibrous structure” is used to refer to extrudates or other products in which macromolecules such as protein fibers and/or cultivated cells are substantially aligned in one direction.

As used herein, the term “fine screen” refers to a screen or mesh with pores having about 35 to 400 mesh (a diameter of about 500 to 27 microns).

As used herein, the term “food product” means any food, feed, snack, food supplement, treat, meal substitute, or meal replacement, whether intended for a human or an animal. Animal food includes food or feed intended for any domesticated or wild species. In preferred embodiments, a food for an animal represents a nutritionally complete food or dietary composition. Examples of such animal foods include extruded pet foods such as foods for canines and felines, e.g., dogs and cats.

As used herein, “hemp” includes all species of the Cannabis plant genus, including without limitation Cannabis sativa, Cannabis indica, and Cannabis ruderalis.

As used herein, the term “high-shear mixer” refers to an apparatus that disperses or transports one phase or ingredient (liquid, solid, or gas) into a main continuous phase (liquid), with which it would normally be immiscible.

As used herein, “metabolic micronutrients” refers to a mixture compounds that are inherently present in a peppercorn and/or a black pepper that modulate one or more physiological processes in a human or animal species. Metabolic micronutrients included, but are not limited to primary and secondary metabolites of plant biochemical pathways, small molecules, vitamins, minerals, amino acids, peptides, proteins, carbohydrates, lipids, fatty acids, sugars, starches, fibers, small molecules, and primary and secondary metabolites.

As used herein, “microparticle” refers to a dry particle in a size range between 50-5000 micron comprised of an agglomerated bioactive substance enrobed in a double layer of emulsifier and solid fats. The microparticles encompass all microparticles of the disclosure, whether they are granules, beads, strands, particles, or any other solid accumulation.

As used herein, “nutraceutical” generally refers to natural, bioactive chemical compounds that provide physiological benefits, including disease prevention and health promotion which may be used to supplement the diet. Nutraceuticals can be either purified or concentrated by using bioengineering methods and can be enhanced through genetic methods, which contain elevated levels of natural substances. Examples of nutraceuticals include isolated nutrients and herbal products and generally contain at least one of the following ingredients: a vitamin, a mineral, an herb or other botanical, an amino acid, a metabolite, constituent, extract, or combination of these ingredients. Common examples of nutraceuticals include beta-carotene, ephedra, Ginko biloba, goldenseal, valerian, ginseng, green tea extract, and echinacea. The nutraceuticals described herein may be useful for maintenance and support of, for example, healthy joints, skin, eye and brain function, heart and circulatory system, and general health.

As used herein, the term “pharmaceutical composition” refers to a mixture of one or more compounds disclosed herein with other chemical components, such as diluents or carriers. The pharmaceutical composition facilitates administration of the compound to an organism. Pharmaceutical compositions will generally be tailored to the specific intended route of administration.

As used herein, the term “shear” means a cutting action that reduces food particle size, increasing its surface area, and therefore, its interaction with enzyme molecules. In some embodiments, high shear is created by circulating the slurry through a high speed, high shear mixer throughout the digest at rates in the range of 105-106 sec- or more.

As used herein, “solvent” is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclic s, and water, among others.

As used herein, the term “weight percent,” when referring to a component, is the weight of the component divided by the weight of the composition that includes the component, multiplied by 100%. For example, the weight percent of component A when 5 grams of component A is added to 95 grams of component B is 5% (e.g., 5 g A/(5 g A+95 g B)×100%).

### Device

An aspect of the disclosure relates to an extruder, and more particularly, to an extruder for mixing, compound, kneading, and/or extruding of biomaterials. In some embodiments, the extruder may be an integrated single screw extruder. In some embodiments, the extruder may be a twin screw extruder.

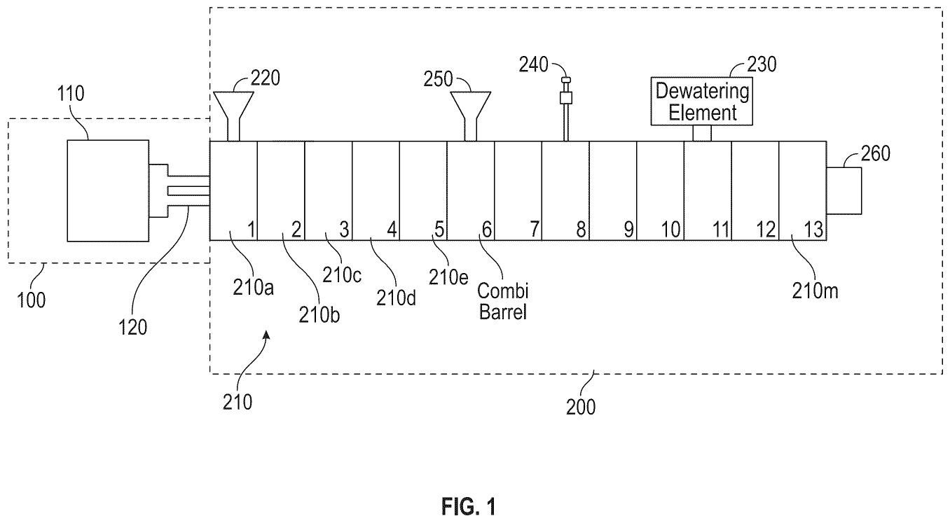

In a first embodiment of the disclosure, an extruder system is provided, including an integrated extruder 100 and a barrel assembly 200, as shown in FIG. 1. The integrated extruder 100 further comprises a twin screw extruder 110 and two co-rotating screws 120. The two co-rotating screws 120 include two co-rotating screws housed within the barrel assembly and having one or more sections. For example, a co-rotating screw may have a first threaded section and a second grinding section and a third shearing section. Another example of a co-rotating screw may have sections that assist in tasks including, but not limited to, mixing, shearing, kneading, grinding, and extracting. The barrel assembly 200 further comprises a plurality of barrels 210, which are connected to each other. For example, the barrel assembly may consist of 13 barrels, where a first barrel 210a may be attached to a second barrel 210b, and the second barrel 210b may be attached to a third barrel 210c, and so on. In some embodiments, the co-rotating screws are housed within the barrels of the barrel assembly 200. In some embodiments, one or more barrels may include heating elements and/or cooling elements. One or more of the barrels may comprise a combi barrel. In some embodiments, a combi barrel comprises a barrel with a hole in the side, which may allow for an additional input or an additional output. In some embodiments, the present extruder system combines a single screw configuration and twin screw configuration into a single extruder system. For example, a first barrel assembly is used to house a twin screw configuration. In some embodiments, the extruder system can be used as a compounder, mixer, reactor, or a combination thereof. In some embodiments, the extruder can be used for processing may types of biomaterials. These biomaterials may include plants, nuts, shells, seeds, biowaste, or combinations thereof. In some embodiments, the barrel assembly 200 can be intermeshed or non-meshed, depending on the specific configuration of the extruder system. In some embodiments, the barrel assembly 200 can further include a kneading block screw element. In some embodiments, the kneading block screw element can be rotated either clockwise or counter clockwise. In some embodiments, the extruder system may further include a conveying compartment. In some embodiments, the extruder system may be designed to convey material fed from a hopper and feed system to the twin screw extruder 110.

In some embodiments, the disclosed systems and methods may produce products with advantageous particle size. In some embodiments, the particle sizes may range from about 250-2000 μm. In some embodiments, the particle size may range from at least one of 500-2000 μm, 250-500 μm, 125-250 μm, and 63-125 μm. In some embodiments, the particle size may range from a high or low point independently selected from any of about 100 μm, about 105 μm, about 140 μm, about 149 μm, about 210 μm, about 354 μm, about 445 μm, about 581 μm, about 595 μm, and about 1000 μm.

In some embodiments, the extruder system may further include one or more heaters. In some embodiments, the extruder system may further include one more cooling systems. The heaters may be designed to heat material conveyed and kneaded within the barrel assembly 200. This may assist in the shearing of the material, as should be understood by those of skill in the art. An option vent and vacuum port can also be provided on the barrel assembly 200. In some embodiments, all or portions of the extruder may be heated in a temperature range from room temperature to 275° C. In some embodiments, temperature ranges according to the disclosure include from about 20° C. to about 275° C., from about 130° C. to about 200° C., from about 130° C. to about 150° C., from about 140° C. to about 165° C., and from about 225° C. to about 275° C. In some embodiments, temperatures according to the disclosure may range from about a high or about a low temperature from any of the following room temperature, 80° C., 85° C., 900 C, 100° C., 120° C., 130° C., 140° C., 150° C., 160° C., 165° C., 180° C., 200° C., 220° C., 225° C., 250° C., and 275° C. In some embodiments, particularly advantageous whole food ingredients and other products may be prepared within temperature ranges disclosed herein. The non-limiting examples of the disclosure provide description of advantageous temperature ranges and operating parameters. One of ordinary skill in the art will contemplate that the impact of temperature and shear on the extruding product may depend on residence time in the extruder, which is influenced by the length and flow rate within the extruder. In some embodiments, extruder screws may be rotated at a speed of rotations per minute (rpm) from a range of about 50 rpm to about 1000 rpm. In some embodiments, the extruder may be operated at any of about 50 rpm, about 70 rpm, about 100 rpm, about 150 rpm, about 160 rpm, about 200 rpm, about 250 rpm, about 300 rpm, about 325 rpm, about 350 rpm, about 400 rpm, about 450 rpm, about 600 rpm, and about 1000 rpm.

In some embodiments, the input to the system may be pre-treated. For example, input ingredients may be roasted or otherwise temperature treated prior to being introduced into the extruder. One of ordinary skill in the art would understand that associated parameters like temperature and RPM may be adjusted to accommodate pre-treatment. Additionally, one of ordinary skill in the art would understand that systems with multiple components may optionally combine or remove any of the disclosed components.

In a second embodiment of the disclosure, the barrel assembly 200 of the first aspect may further comprise a first feeder 220. For example, the first feeder 220 may include a first feeder 220 positioned adjacent to barrel 1 210a and allow material to be input into the barrel assembly 200 via barrel 1 210a.

In third aspect of the disclosure, the barrel assembly 200 of the second aspect may further comprise a second feeder 250. For example, the second feeder 250 may further comprise barrel 6 210f being a combi barrel used to side feed material (e.g., starch, flour, or other protein) into the barrel assembly 200 via a second feeder 250 (ZS-B25).

In a fourth aspect of the disclosure, the barrel assembly 200 of the first aspect may further include a dewatering element 230. For example, barrel 11 210k might be designated as the dewatering barrel. In a first example, the dewatering element 230 may further comprise a combi barrel, for example barrel 11 210k, along with a side feeder with an open barrel set upside down for the purpose of dewatering. In a second example, the dewatering element 230 may comprise using an open vacuum dome attached to a valve attached to barrel 11 210k. In a third example, the dewatering element 230 may comprise using an open vacuum dome attached directly to the open barrel 11 with a 40 mesh screen inserted between the open barrel and the open vacuum dome.

In a fifth aspect of the disclosure, the barrel assembly 200 of the first aspect may further comprise a die plate 260. For example, the die plate 260 may consist of an 8 mm×2 mm die insert placed at the end of the barrel assembly 200.

In a sixth aspect of the disclosure, the barrel assembly 200 of the first aspect may further comprise a pressure released opening (not shown). For example, the pressure release opening may consist of an opening, or some sort of pressure release valve, on or attached to barrel 4, that constrains the pressure inside barrel 4 to atmospheric pressure.

In a seventh aspect of the disclosure, the barrel assembly 200 of the first aspect may further comprise a water injection element 240. For example, the water injection element 240 may be attached to barrel 7, or to barrel 3, or to barrel 9.

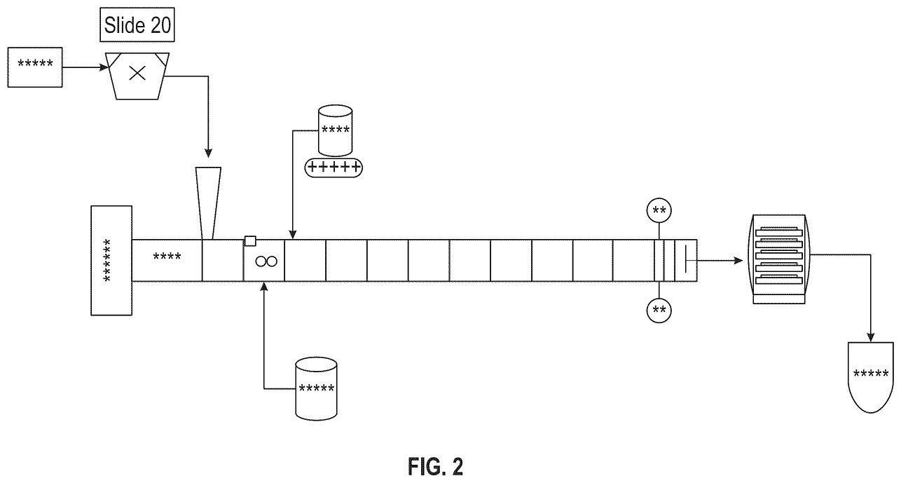

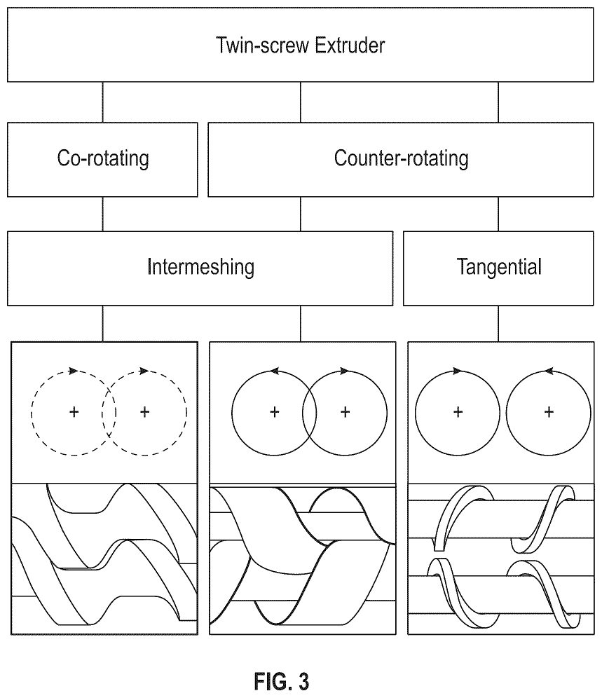

In another aspect of the disclosure, an extruder system is provided in FIG. 2. FIG. 3 illustrates a perspective view of twin screws of an aspect of the disclosure.

“Extrusion” is a process used to create objects of a fixed cross-sectional profile. A material may be pushed or pulled through a die of the desired cross-section. Two advantages of this process over other manufacturing processes include the ability to create very complex cross-sections, and to prepare products that are brittle, because the material passing through an extruder will generally only encounter compressive and shear stresses. High-moisture extrusion is also known as wet extrusion. Extruders might traditionally comprise an extruder barrel within which rotates a close-fitting screw. A screw may be made up of screw elements, some of which can be helical screw threads to move material through the extruder barrel.

During extrusion, the material is introduced into the extruder barrel toward one end, moved along the extruder barrel by the action of the screw, and may be forced out of the extruder barrel through a nozzle or die at the other end. The rotating screw mixes and works the material in the barrel and compresses it to force it through the die or nozzle. The degree of mixing and work to which the material is subjected, the speed of movement of the material through the extruder barrel and thus the residence time in the extruder barrel, and the pressure developed in the extruder barrel can be controlled by, for example, the pitch of the screw thread elements, the speed of rotation of the screw and the rate of introduction of material into the extruder barrel.

An extruder barrel may comprise multiple extruder barrel sections which are joined end to end. Multiple extruder barrel sections may be included to carry out different processes involved in extrusion, such as conveying, kneading, mixing, devolatilizing, and metering. Each extruder barrel section comprises a liner which is press fit into an extruder barrel casing, and heating and cooling elements are provided to regulate the temperature of the extruder barrel section within the permissible range. The total length of an extrusion process can be defined by the total length of the modular extrusion barrel(s). An extruder barrel may be quantified by its diameter. A “cooling die” may be used to controllably cool the extruded product to a desired temperature, such as room temperature or a freezing temperature.

By way of an example, cold extrusion may be used to gently mix and shape dough, without direct heating or cooking within the extruder. In food processing, cold extrusion is used mainly for producing raw pasta and dough. These products can then be subsequently processed: dried, baked, vacuum-packed, and or frozen.

Hot extrusion may be used to thermomechanically transform raw materials in a short time with high temperature conditions and pressure. In food processing, hot extrusion may be used to cook biopolymer-based raw materials to produce textured food and feed products, such as ready-to-eat breakfast cereals, snacks (savory and sweet), pet foods, feed pellets, and other similar food products. Hot extrusion may include, for example, melting and/or plasticization of the ingredients, gelatinization of starch and denaturation of proteins. Heat can be applied through, for example, steam injection, external heating of the barrel, and or mechanical energy. The material going through the extruder can be pumped, shaped and also expanded. When expanding or puffing, a porous and fibrous texture is usually formed, and usually, at the same time, can partially dehydrate the food product. The shape and size of the final product may be varied by using different die configurations. Extruders can be used to make products with little expansion (such as pasta), moderate expansion (such as shaped breakfast cereal, meat substitutes, breading substitutes, modified starches, pet foods (soft, moist, and dry)), or a great deal of expansion (such as puffed snacks, puffed curls and balls).

### Processes

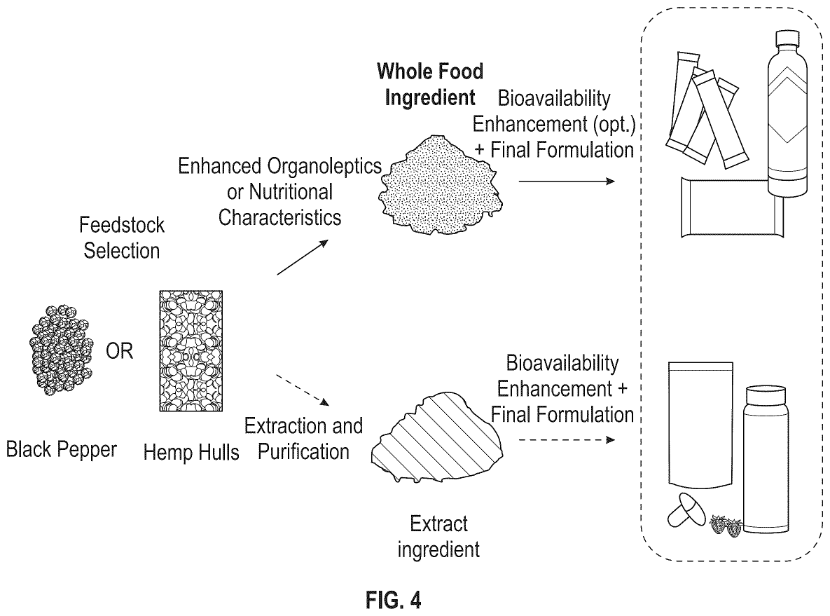

An aspect of the disclosure provides for a process of producing food products and formulations. FIG. 4 illustrates an embodiment of a process and product pathways for a whole food ingredient or an extract ingredient.

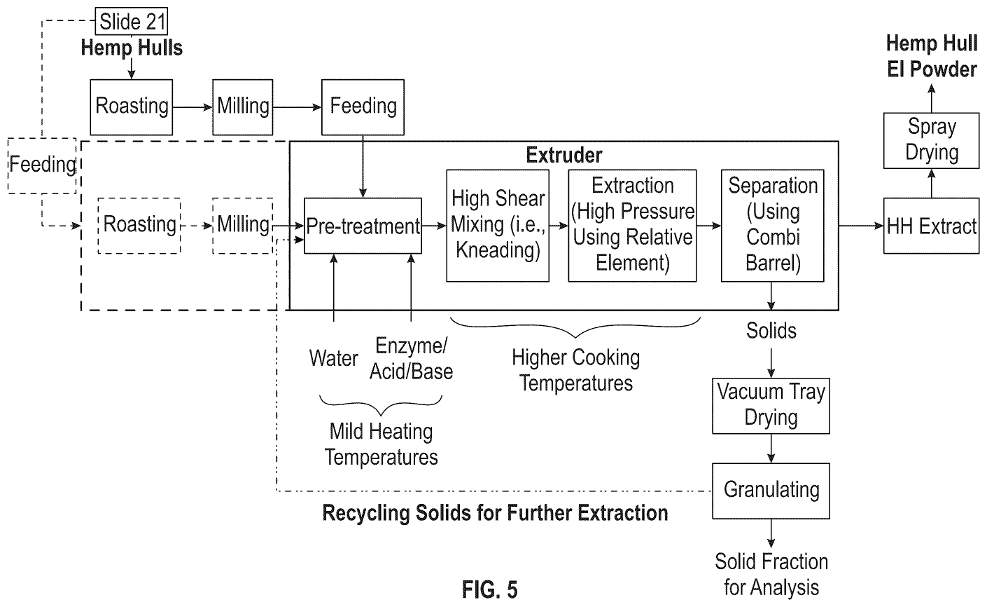

FIG. 5 illustrates a flow chart of an embodiment of the disclosure to produce a hemp hull extract powder. In some embodiments, hemp hulls are first subjected to roasting, followed by milling, followed by feeding into an extruder of the disclosure. In some embodiments, the hemp hulls in the extruder may be subjected to a pre-treatment of water, enzymes, acid, base, heat, or a combination thereof. In some embodiments, the hemp hulls may be subjected to high shear mixing. In some embodiments, the hemp hulls may be subjected to high pressure in an extraction element. In some embodiments, the hemp hulls may be further subjected to a separation process.

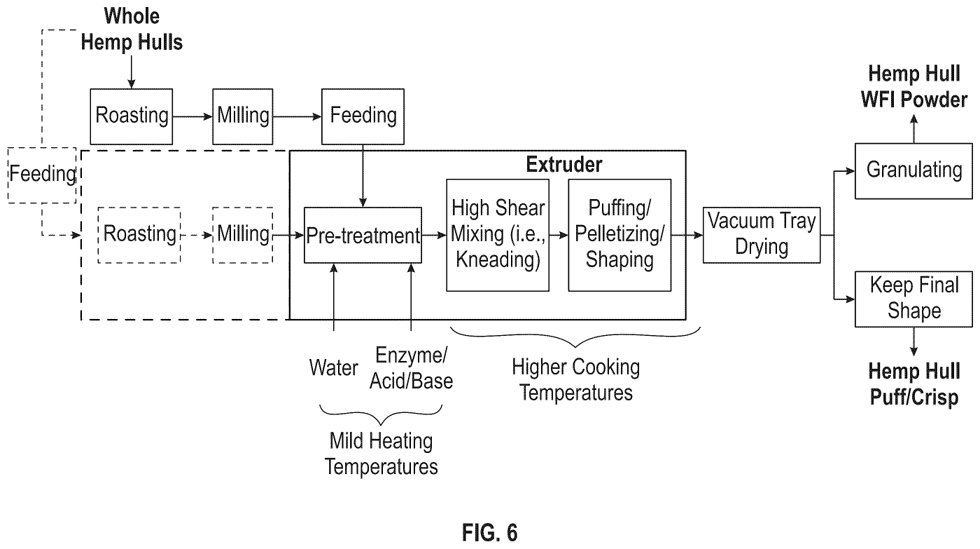

FIG. 6 illustrates a flow chart of an embodiment of the disclosure to produce hemp hull whole food ingredient powder or hemp hull puff/crisp.

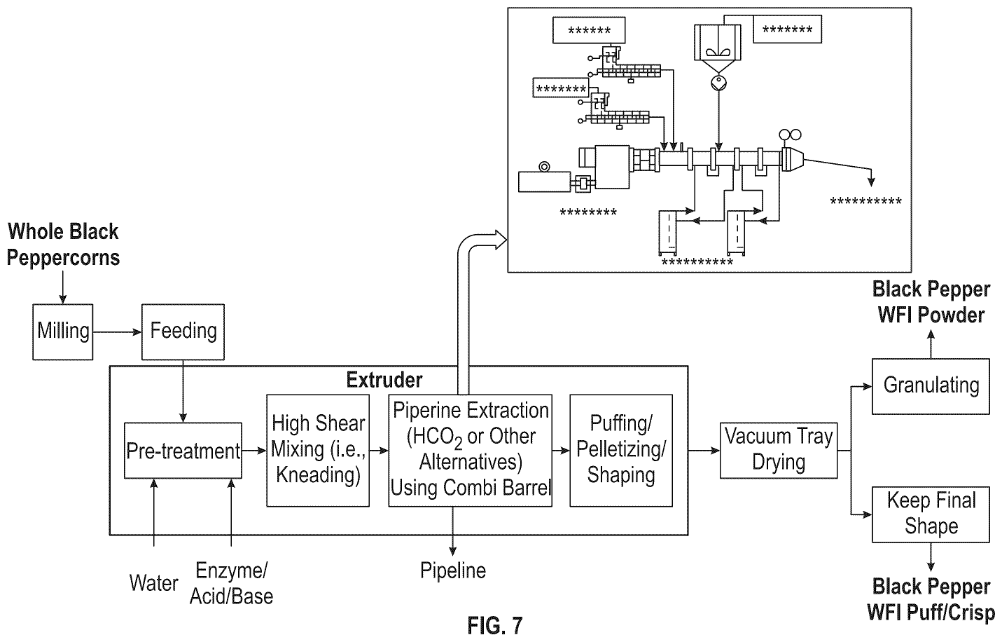

FIG. 7 illustrates a flow chart of an embodiment of the disclosure to produce black pepper whole food ingredient powder or black pepper puff/crisp.

In some embodiments, the one or more processes may include conventional processes. In some embodiments, the conventional processes are batch processes. In some embodiments, the batch processes may include stepwise processes for each unit operation requiring different equipment. Such processes may include supercritical CO₂, high pressure water extraction, roasting, mixing, milling, enzymatic and pH hydrolysis, drying, etc. In some embodiments, the method includes a combination of one or more of high heat, high pressure, and high shear.

The disclosure includes methods for producing a food composition comprising a whole food ingredient made by any of the methods as disclosed herein, which may then be used to mix with other edible components to provide food compositions as disclosed herein. Additionally, the disclosure includes a food composition for human or animal consumption, comprising a whole food ingredient, wherein the food product may be at least 50% (w/w) protein on a dry weight basis.

Whole food ingredients as disclosed herein may be used to create a number of food compositions, including, without limitation, a number of new food compositions reaction flavors, dairy alternative products, ready to mix beverages and beverage bases; extruded and extruded/puffed products; textured products such as meat analogs; sheeted baked goods; meat analogs and extenders; bar products and granola products; baked goods and baking mixes; granola; and soups/soup bases.

Methods to prepare a food product may include additional, optional steps of cooking, extruding, and/or puffing the food composition according to methods known in the art to form the food compositions comprising a whole food ingredient. The disclosure includes methods to make food compositions, comprising providing a whole food ingredient as described herein, providing an edible material, and mixing the whole food ingredient and the edible material. The edible material may be, without limitation, a starch, a flour, a grain, a lipid, a colorant, a flavorant, an emulsifier, a sweetener, a vitamin, a mineral, a spice, a fiber, a protein powder, nutraceuticals, sterols, isoflavones, lignans, glucosamine, an herbal extract, xanthan, a gum, a hydrocolloid, a starch, a preservative, a legume product, a food particulate, and combinations thereof. A food particulate may include cereal grains, cereal flakes, crisped rice, puffed rice, oats, crisped oats, granola, wheat cereals, protein nuggets, texturized plant protein ingredients, flavored nuggets, cookie pieces, cracker pieces, pretzel pieces, crisps, soy grits, nuts, fruit pieces, corn cereals, seeds, popcorn, yogurt pieces, and combinations of any thereof.

Extrusion may be advantageously used to process whole food ingredients with or without high protein substances, while controlling the functional properties such as density, rate and time of rehydration, shape, product appearance and mouthfeel.

A general procedure may be as follows and may be modified as known in the art. A flour mix may be prepared, and the dry ingredients are blended together in the premixture stage. In the optional preconditioning step (in a section of an extruder device known as a preconditioner) the steam and water are usually added at this stage to wet/moisten and warm the flour mix. Generally, the starch and protein are plasticized using heat, pressure and/or mechanical shear, then realigned and expanded as the mixture exits the extruder. The material coming from the extruder moisture may range from 1% to 50%, from 5% to 40%, from 10% to 35%, from 15% to 30%, and from 20% to 30%. Optionally, this extruded material may be dried to about 3%-5% moisture or less in a dryer portion. Methods to prepare a food composition may include the additional, optional steps of roasting, cooking, extruding, and/or puffing the food composition according to methods known in the art to form the food compositions comprising the whole food ingredient of the disclosure. Cooling may then optionally be performed to lower the temperature of the dried product to ambient conditions, followed by an optional packaging step.

Advantageously, according to this disclosure, whole food ingredients may improve the puffed products' drying process, by either the drying rate or the total amount of drying. Unexpectedly, food products of this disclosure are observed to tolerate both high levels of a whole food ingredient and high levels of protein while retaining structural integrity. While a manufacturer may typically vary the concentrations of various ingredients in a food product, food products of this disclosure were observed to tolerate surprising levels of protein, including at levels outside of what was known in the art. Also, unexpectedly, even a high amount of a whole food ingredient produced good puffed products that did not have inhibited puffing. Additionally, according to this disclosure, a whole food ingredient was observed to be easily combined with most protein combinations, even with soy protein. Unexpectedly, a high amount of a whole food ingredient may be helping the puffing action (enhancing). In some embodiments, a food product may be a puffed product with a high amount of a whole food ingredient.

An aspect of the disclosure includes processes for preparing a food product, whole food ingredient, or extract ingredient.

In some embodiments, a process for preparing a food product includes roasting hemp hulls to form roasted hemp hulls, milling roasted hemp hulls to form milled roasted hemp hulls, and feeding milled roasted hemp hull into an integrated extruder of form a food product as described herein. In some embodiments, the process further includes pre-treating the milled roasted hemp hulls with water, enzymes, acid, base, or a combination there. In some embodiments, the process further comprising high shear mixing the milled roasted hemp hulls. In some embodiments, the process further includes puffing, pelletizing or shaping the milled roasted hemp hulls. In some embodiments, the food process further includes vacuum drying the milled roasted hemp hulls. In some embodiments, the process includes granulating the milled roasted hemp hulls.

In some embodiments, a process for preparing a whole food ingredient includes roasting hemp hulls to form roasted hemp hulls, milling roasted hemp hulls to form milled roasted hemp hulls, and feeding milled roasted hemp hull into an integrated extruder of form a food product as described herein. In some embodiments, the process further includes pre-treating the milled roasted hemp hulls with water, enzymes, acid, base, or a combination there. In some embodiments, the process further comprising high shear mixing the milled roasted hemp hulls. In some embodiments, the process further includes puffing, pelletizing or shaping the milled roasted hemp hulls. In some embodiments, the food process further includes vacuum drying the milled roasted hemp hulls. In some embodiments, the process includes granulating the milled roasted hemp hulls.

In some embodiments, a process for preparing an extract ingredient includes roasting hemp hulls to form roasted hemp hulls, milling roasted hemp hulls to form milled roasted hemp hulls, and feeding milled roasted hemp hull into an integrated extruder of form a food product as described herein. In some embodiments, the process further includes pre-treating the milled roasted hemp hulls with water, enzymes, acid, base, or a combination there. In some embodiments, the process further comprising high shear mixing the milled roasted hemp hulls. In some embodiments, the process further includes puffing, pelletizing or shaping the milled roasted hemp hulls. In some embodiments, the food process further includes vacuum drying the milled roasted hemp hulls. In some embodiments, the process includes granulating the milled roasted hemp hulls.

In some embodiments, the integrated extruder has a screw speed from about 2 Hz, 4 Hz, 6 Hz, 8 Hz, 10 Hz, 12 Hz, 14 Hz, 16 Hz, 18 Hz, 20 Hz, 22 Hz, 24 Hz, 26 Hz 28 Hz, 30 Hz, 32 Hz, 34 Hz, 36 Hz, 38 Hz, 40 Hz or ranges including, between, and/or spanning the aforementioned values. In some embodiments, the integrated extruder has a screw speed from about 22 Hz.

In some embodiments, the hemp hulls are added at a steady state. In some embodiments, the hemp hulls are added at a rate from about 1 Hz, 3 Hz, 5 Hz, 7 Hz, 9 Hz, 11 Hz, 13 Hz, 15 Hz, 17 Hz, 19 Hz, 21 Hz or ranges including, between, and/or spanning the aforementioned values. In some embodiments, the hemp hulls are added at a rate from about 13 Hz. In some embodiments, the hemp hulls are added at speed from about 50 rpm, 75 rpm, 100 rpm, 125 rpm, 150 rpm, 175 rpm, 200 rpm, 225 rpm, 250 rpm, 275 rpm, 300 rpm, 325 rpm, 350 rpm, 375 rpm, 400 rpm, 425 rpm, 450 rpm, 475 rpm, 500 rpm, to about 750 rpm, 1000 rpm or ranges including, between, and/or spanning the aforementioned values. In some embodiments, the hemp hulls are added at speed from about 500 rpm to about 700 rpm.

In some embodiments, the process further includes adding water. In some embodiments, water is added at a rate from about 0.01 lbs/hr, 0.02 lbs/hr, 0.05 lbs/hr, 0.1 lbs/hr, 0.15 lbs/hr, 0.2 lbs/hr, 0.25 lbs/hr, 0.3 lbs/hr, 0.35 lbs/hr, 0.4 lbs/hr, 0.45 lbs/hr, 0.5 lbs/hr, 0.55 lbs/hr, 0.6 lbs/hr, 0.65 lbs/hr, 0.7 lbs/hr, 0.75 lbs/hr, 0.8 lbs/hr, 1.0 lbs/hr, 2.0 lbs/hr, 3.0 lbs/hr, 4.0 lbs/hr, 5.0 lbs/hr, 6.0 lbs/hr, 7.0 lbs/hr, 8.0 lbs/hr, 9.0 lbs/hr, 10.0 lbs/hr or ranges including, between, and/or spanning the aforementioned values. In some embodiments, water is added at a rate from about 0.4 lbs/hr. In some embodiments, water is added at a rate from about 5 lbs/h to about 10 lbs/h.

In some embodiments, the roasting hemp hulls may include roasting at one or more temperatures. In some embodiments, roasting hemp hulls is at a temperature range from about 100° C., 110° C., 120° C., 130° C., 140° C., 150° C., 100° C., 160° C., 170° C., 180° C., 190° C., 200° C., 210° C., 210° C., 220° C., 230° C., 240° C., 250° C., 260° C., 270° C., 280° C., 290° C., 300° C. or ranges including, between, and/or spanning the aforementioned values. In some embodiments, roasting hemp hulls is at a temperature range from about 100° C. to about 210° C. In some embodiments, roasting hemp hulls is at a temperature range from about 130° C. to about 180° C. In some embodiments, roasting hemp hulls is at a temperature range from about 160° C. to about 210° C. In some embodiments, roasting hemp hulls is at a temperature range from about 225° C. to about 275° C. In some embodiments, roasting hemp hulls is at a temperature range from about 130° C. In some embodiments, roasting hemp hulls is at a temperature range from about 160° C. In some embodiments, roasting hemp hulls is at a temperature range from about 180° C. In some embodiments, roasting hemp hulls is at a temperature range from about 210° C.

In some embodiments, the milling roast hemp hulls is at speed from about 50 rpm, 75 rpm, 100 rpm, 125 rpm, 150 rpm, 175 rpm, 200 rpm, 225 rpm, 250 rpm, 275 rpm, 300 rpm, 325 rpm, 350 rpm, 375 rpm, 400 rpm, 425 rpm, 450 rpm, 475 rpm, 500 rpm, to about 750 rpm, 1000 rpm or ranges including, between, and/or spanning the aforementioned values. In some embodiments, the milling roast hemp hulls is at speed from about 150 rpm to about 250 rpm.

In some embodiments, a method is provided for making a liquid product. In some embodiments, the method includes adding hemp hulls to an extruder at a speed from about 500 rpm to about 700 rpm, wherein the hemp hulls are added at a rate from about 3 lb/h to about 8 lb/h, wherein water is added from about 5 lb/h to about 10 lb/h, wherein the temperature is from about 130° C. to about 150° C., and screening the hemp hulls with a mesh screen, thereby collecting the liquid product.

In some embodiments, a method is provided for making an expanded food product. In some embodiments, the method includes forming a dough by admixing water and hemp hulls, extrusion cooking the dough to form a cooked dough, extruding the cooked dough through an extruder to form cooked dough pieces, and cutting the cooked dough pieces, wherein the cooked dough pieces retain one or more bioactives.

In some embodiments, a method is provided for producing a hemp hull containing composite food product. In the method includes extruding hemp hulls with an extruder, thereby forming a milled hemp hull, and adding the milled hemp hull to addition food ingredients, thereby producing a hemp hull containing composite food product.

### Food Products & Formulations

An aspect of the disclosure includes a food product. In some embodiments, the food product is derived from hemp hulls. In some embodiments, the food product is a solid food. In some embodiments, the food product is a semi-solid food. In some embodiments, the food product is a spread.

In some embodiments, the food product may comprise additional ingredients, such as sugar, simple and complex carbohydrates, flour, protein, fats, preservatives, artificial coloring, artificial flavoring, oils, emulsifiers, flavor enhancers, starches and grains.

In some embodiments, the food product includes a carbohydrate. A variety of carbohydrates are used in food products, such as various sugars and starches. Carbohydrates are an important source of energy for the body, including complex carbohydrates (like whole grains and vegetables) and simple carbohydrates (like sugar and refined grains). There are several common carbohydrates that are used in food products, including: starch, sugar, fructose, maltodextrin, dextrose, corn syrup, oligosaccharides, cellulose, complex carbohydrates, such as inulin that may be added as a prebiotic fiber in processed foods. According to the disclosure, the concentration of carbohydrates may vary depending on the intended use of a product. In some embodiments, carbohydrates may comprise from about 1% by weight to about 95% by weight of a food product. In some embodiments, carbohydrates may comprise from about 1% by weight to about 95% by weight of a food product. In some embodiments, a food product may comprise carbohydrates in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, dietary fiber may be present as a primary or secondary ingredient. For example it may be present as a hemp product, or added as a secondary ingredient. Fiber ingredients suitable for use herein include, but are not limited to, powdered cellulose, carboxylmethyl cellulose, oat bran, corn bran, wheat bran, rice bran, barley bran, Hemi cellulose, powdered cellulose, carboxymethyl cellulose, oat bran, corn bran, wheat bran, rice bran, inulin, sugar beet fiber, Fructo-oligo-saccarides (“FOS”) such as inulin, Guar Gum, Gum Arabicum, fiber from fruits (such as apples, oranges, grapes, berries, or other fruits) or fruit fiber, and mixtures. According to the disclosure, the concentration of dietary fiber may vary depending on the intended use of a product. In some embodiments, dietary fiber may comprise from about 1% by weight to about 95% by weight of a food product. In some embodiments, dietary fiber may comprise from about 1% by weight to about 95% by weight of a food product. In some embodiments, a food product may comprise dietary fiber in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include a sugar. Sugars, such as high fructose corn syrup, sucrose, glucose syrup, dextrose, and other sweeteners are often added to processed foods to make them more palatable. Additionally, sugar alcohols or maltodextrins, may be added to food products to reduce caloric content and/or to enhance nutritional profile of the product. In some embodiments, a food product may comprise one or a plurality of saccharides that are slowly or incompletely digested by humans, if not totally indigestible. These sugars can include isomaltose, panose and branched oligomers having a degree of polymerization of four or greater. Additional non-limiting examples of sugars include sucrose, HFCS, fructose, brown sugar (which can be either partially or fully refined), powdered sugar (also known as confectioner's sugar), high fructose corn syrup, honey, molasses, maple syrup, agave nectar, coconut sugar, date sugar, fruit juice concentrates, maltodextrin, dextrose, glucose syrup, maple syrup, molasses, and lactose. According to the disclosure, the concentration of sugars may vary depending on the intended use of a product. In some embodiments, sugar may comprise from about 1% by weight to about 95% by weight of a food product. In some embodiments, the sugar(s) component comprises from about 2% to about 10% by weight of the composition. In some embodiments, sugars may comprise from about 1% by weight to about 95% by weight of a food product. In some embodiments, a food product may comprise sugars in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include a starch. In some embodiments, food products may additionally comprise a starch ingredient(s) in amounts sufficient to provide about 5% to 45%, or about 10%-30%, or about 15%-25 starch in the food products. Starchy components may include not only pure added cereal flours or other granulations but also any starchy fraction provided by other ingredients such as oat bran or soy protein. In some embodiments, a starch may comprise any conventionally employed starch or cereal flour ingredient, for use in a ready-to-eat cereal. Exemplary suitable starchy cereals include cereal flours from major cereal grains including wheat, rice, corn (maize), oats, barley, rye, or starch fractions isolated from the cereal flowers including, for example cornstarch, wheat starch, rice starch, and various treated starches including pre-gelatinized starches and/or modified starches.

In some embodiments, the food product may include a flour. Several types of flour may be used according to the disclosure. For example, common types of flour used in food products, include all-purpose flour (which like most other flour may include protein content), Whole wheat flour, Bread flour, Cake flour, pastry flour, self-rising flour, and gluten-free flour. According to the disclosure, the concentration of flour may vary depending on the intended use of a product. For example, in baked goods like bread, cakes, and cookies, flour is typically a main ingredient and may be included at concentrations ranging from 50% to 100% of the total dry ingredients. In soups and sauces, flour is often used as a thickener and may be included at concentrations ranging from 1% to 5% of the total ingredients. Batter and breading: In fried foods like chicken or fish, flour is often used as part of the batter or breading and may be included at concentrations ranging from 20% to 50% of the total dry ingredients. In snack foods, like crackers and chips, flour may be included at concentrations ranging from 30% to 70% of the total ingredients. In some embodiments, flour may comprise from about 1% by weight to about 95% by weight of a food product. In some embodiments, flours may comprise from about 1% by weight to about 95% by weight of a food product. In some embodiments, a food product may comprise flour in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include a fat or a fat source. In some embodiments, the fat or fat source may include an oil. In some embodiments, the food product may include one or more fats or oils. Several types of fats and oils may be used according to the disclosure. For example, common types of fats include butter, margarine, vegetable oils, shortening, and lard. According to the disclosure, the concentration of fats may vary depending on the intended use of a product. In baked goods like cakes, cookies, and pastries, fats like butter, shortening, or oil are often used to provide moisture, flavor, and texture. In some embodiments, Fats may be included at concentrations ranging from 10% to 30% of the total ingredients. In some embodiments, like in sauces and dressings, fats like olive oil or mayonnaise are often used to provide flavor and texture. In some embodiments, Fats may be included at concentrations ranging from 10% to 30% of the total ingredients. In fried foods like chicken or French fries, fats like vegetable oil or lard are used for frying and may be included at concentrations ranging from 30% to 60% of the total ingredients. In snack foods like chips and crackers, fats like vegetable oil or palm oil are often used to provide flavor and texture. In some embodiments, Fats may be included at concentrations ranging from 10% to 30% of the total ingredients. In some embodiments, fats may comprise from about 1% by weight to about 95% by weight of a food product. In some embodiments, fats may comprise from about 1% by weight to about 95% by weight of a food product. In some embodiments, a food product may comprise fat in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include a protein. Proteins may be included in a range of concentrations depending on the product. Protein sources can include soy protein, soy flour, soy protein isolate, whey protein isolate, casein, gelatin, legume protein isolates, soy protein concentrate, egg albumin or egg white, wheat protein concentrate, legume protein concentrates and mixtures thereof. In some embodiments, meat products like beef jerky, sausages, or meatballs, may include proteins as a primary ingredient. Proteins may be included at concentrations ranging from about 10% to about 25% of the total ingredients. In dairy products like cheese, yogurt, or milk, proteins like casein or whey may be the primary ingredient. Proteins may be included at concentrations ranging from about 3% to about 15% of the total ingredients. In plant-based products like tofu, tempeh, or seitan, proteins from soy, peas, legumes, or wheat are may be primary ingredient. Proteins may be included at concentrations ranging from about 10% to about 25% of the total ingredients. In energy bars and protein bars, proteins like whey or soy, or any protein according to the disclosure may be added to provide a source of protein. Proteins may be included at concentrations ranging from about 10% to about 25% of the total ingredients. According to the disclosure, the concentration of proteins may vary depending on the intended use of a product. In some embodiments, proteins may comprise from about 1% by weight to about 95% by weight of a food product. In some embodiments, proteins may comprise from about 1% by weight to about 95% by weight of a food product. In some embodiments, a food product may comprise proteins in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, a food product may comprise an emulsifier. Emulsifiers like lecithin or mono- and diglycerides may be used to keep the oil and vinegar or other ingredients from separating. Emulsifiers may be included at concentrations ranging from 0.5% to 2% of the total ingredients. In baked goods like cakes and bread, emulsifiers like monoglycerides or polysorbates are used to improve the texture and extend the shelf life. Emulsifiers may be included at concentrations ranging from 0.5% to 2% of the total ingredients. In dairy products like ice cream and yogurt, emulsifiers like carrageenan or sodium alginate are used to prevent the ingredients from separating and to improve texture. Emulsifiers may be included at concentrations ranging from 0.5% to 2% of the total ingredients. According to the disclosure, the concentration of sugars may vary depending on the intended use of a product. In some embodiments, a food product may comprise an emulsifier in a range with high and low values independently selected from about 0.01%, 0.03%, 0.05%, 0.08%, 0.010%, 0.015%, 0.02%, 0.50%, 0.55%, 0.60%, 0.65%, 0.70%, 0.75%, 0.80%, 0.85%, 0.90%, 1.0%, 1.2%, 1.5%, 1.75%, 2%, or ranges including, between, and/or spanning the aforementioned values.

In some embodiments, the food product includes coloring. In some embodiments, the coloring is a coloring agent, color stabilizer, or color enhancer. In some embodiments, the coloring is a food safe ingredient. The term “coloring agent” as used herein refers to a compound that confers a color on a food product. The term “color stabilizer” as used herein refers to a compound that stabilizes a coloring agent or the perception of the color conferred by the coloring agent by the visual system. The term “color enhancer” as used herein refers to a compound that intensifies the coloring conferred by a coloring agent or the perception of such coloring. Examples of suitable coloring agents include but are not limited to artificial colorants, natural colorants, natural extracts (e.g., beet root extract, pomegranate fruit extract, cherry extract, carrot extract, red cabbage extract, red seaweed extract), modified natural extracts, natural juices (e.g., beet root juice, pomegranate fruit juice, cherry juice, carrot juice, red cabbage juice, red seaweed juice), modified natural juices, FD&C (Food Drug & Cosmetics) Red No. 3 (erythrosine), FD&C Green No. 3 (fast green FCF), FD&C Red No. 40 (allura red AC), FD&C Yellow No. 5 (tartazine), FD&C Yellow No. 6 (sunset yellow FCF), FD&C Blue No. 1 (brilliant blue FCF), FD&C Blue No. 2 (indigotine), titanium oxide, annatto, anthocyanins, betanins, beta-APE 8 carotenal, beta-carotene, black currant, burnt sugar, canthaxanthin, caramel, carmine/carminic acid, cochineal extract, curcumin, lutein, carotenoids, monascin, paprika, riboflavin, saffron, turmeric, and combinations thereof. Examples of suitable color stabilizers include but are not limited to antioxidants (e.g., ascorbic acid, vitamin E, rosemary extract, tocopherols, gluconate, metal ions), pH and/or ionic strength adjusting agents (e.g., sodium bicarbonate, potassium bicarbonate), chelating agents (e.g., EDTA), reactive metal complexes (ferric and ferrous salts [e.g., chloride, phosphate, citrate], zinc, copper, magnesium, and manganese), anti-microbial agents (e.g., citrus fruit extract), and combinations thereof. Examples of suitable color enhancers include but are not limited to co-pigments (i.e., non-colored compounds that bind coloring agents creating more color than unbound coloring agents [e.g., non-flavonoid phenols, flavonols, arginine]), metal ions (e.g., ferric salts), metal ion complexes, transition metal complexes, polyphenols, and combinations thereof. In some embodiments, a food product may include a coloring in a range with high and low values independently selected from about 0.1%, 0.2%, 0.3%, 0.4%, 0.5%, 0.6%, 0.7%, 0.8%, 0.9%, 1%, 1.2%, 1.4%, 1.6%, 1.8%, 2.0%, 2.2%, 2.4%, 2.6%, 2.8%, 3% or ranges including, between, and/or spanning the aforementioned values.

In some embodiments, the food product includes ash. In some embodiments, a food product may include ash in a range with high and low values independently selected from about 1%, 2%, 3%, 4%, 5%, 6%, 7%, 8%, 9%, 10% (w/w) or ranges including, between, and/or spanning the aforementioned values. In some embodiments, the food product includes ash from about 2.5% (w/w). In some embodiments, the food product includes ash from about 3.5% (w/w). In some embodiments, the food product includes ash from about 5.5% (w/w).

In some embodiments, the food product includes a carbohydrate to fiber ratio from about 5:1, 4:5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5 (w/w) or ranges including, between, and/or spanning the aforementioned values. In some embodiments, the fiber is dietary fiber. In some embodiments, the food product includes a carbohydrate to dietary fiber ratio from about 2:1. In some embodiments, the food product includes a carbohydrate to dietary fiber ratio from about 1:1. In some embodiments, the food product includes a carbohydrate to dietary fiber ratio from about 1:2.

In some embodiments, the food product may include one or more additional ingredients. If desired, the disclosed food products may additionally include a variety of materials designed to improve their aesthetic or nutritional qualities. These adjuvant materials can include vitamin and/or mineral fortification, colors, flavors, sweetener(s), and mixtures thereof. The precise ingredient concentration may vary. Generally, however, such materials can each comprise about 0.01% to about 5%, preferably about 0.1% to 2% dry weight of a food product. One especially useful material is common salt. In some embodiments, salt comprises about 0.1 to 5%, or about 0.5 to 4.0% of the food products.

In some embodiments, the food product is a liquid product. In some embodiments, the liquid product is produced from hemp hulls extruded by a process described herein. In some embodiments, the liquid product is a beverage. In some embodiments, the liquid product may be incorporated into a milk-based beverage. In some embodiments, the liquid product may be incorporated into a sports drink beverage. In some embodiments, the liquid product may be incorporated into a fruit juice beverage. In some embodiments, the liquid product may be incorporated into an alcoholic beverage. In some embodiments, the liquid product may be incorporated into a water-based beverage. In some embodiments, the liquid product may be a smoothie.

In some embodiments, the food product is a whole food ingredient. In some embodiments, the whole food ingredient is produced from hemp hulls extruded by a process as described herein.

In some embodiments, the whole food ingredient produced from a process as described herein is added as an emulsifier in a food product or formulation. In some embodiments, the whole food ingredient produced from a process as described herein is added as a stabilizer in a food product or formulation. In some embodiments, the whole food ingredient.

An aspect of the disclosure is directed to extruded and/or puffed products and/or cooked products comprising a whole food ingredient. Extruded and/or puffed ready-to-eat breakfast cereals and snacks such as crisps or scoops and pasta noodles are known in the art. Extrusion processes are generally well known in the art, though some advantageous properties of the whole food ingredients were surprisingly found here. For example, the superior processing ability of whole food ingredients according to the present disclosure may include formulations and processing outside of the expected ranges of processing times, ingredients, and concentrations.

In some embodiments, the food product is a pressed cake. In some embodiments, the pressed cake is produced from hemp hulls extruded by a process as described herein. In some embodiments, the pressed cake may be incorporated into a food bar. For example, but not limited to, granola bars, protein bars, candy bars, etc.

In some embodiments, the food product is an extruded puff. In some embodiments, the extruded puff is produced from hemp hulls extruded by a process as described herein.

In some embodiments, the food product is a cereal. In some embodiments, the cereal is produced from hemp hulls extruded by a process as described herein. In some embodiments, the cereal is a breakfast cereal. In some embodiments, the cereal is combined with granola. In some embodiments, the cereal is combined with oatmeal.

In some embodiments, the food product is a bakery product. In some embodiments, the cracker is produced from hemp hulls extruded by a process as described herein. In some embodiments, the bakery product is a cracker. In some embodiments, the bakery product is a bread. In some embodiments, the bakery product is a donut. In some embodiments, the bakery product is a bagel. In some embodiments, the bakery product is a pastry. In some embodiments, the bakery product is a cake.

In some embodiments, the food product is a crisp. In some embodiments, the crisp is produced from hemp hulls extruded by a process as described herein.

### Grain Products

The food product produced using the methods described herein may be in the form of crunchy curls, puffs, chips, crisps, crackers, wafers, flat breads, biscuits, crisp breads, protein inclusions, cones, cookies, flaked products, fortune cookies, etc. The food product may also be in the form of pasta, such as dry pasta or a ready-to-eat pasta. The product may be used as or in a snack food, cereal, or may be used as an ingredient in other foods such as a nutritional bar, breakfast bar, or breakfast cereal. In a pasta, the whole food ingredient may be, in a non-limiting example, be used in levels of about 10 g per 60 g serving (about 20%).

In some embodiments, a protein crisp includes a whole food ingredient as produced by a process as described herein.

A whole food ingredient, as disclosed herein, may be used to supplement dietary fiber of a food product, such as a grain product. Dietary fiber is an essential part of any healthy diet because of its ability to maintain gut function. But today, only 5% of Americans consume the recommended daily intake. A whole food ingredient, as described herein, may include bioactive compounds. A whole food ingredient, as described herein, may include bioactive compounds that have shown the ability to strengthen the gut lining in preclinical studies. A whole food ingredient, as disclosed herein, may comprise a unique gut strengthening combination of both dietary fiber and bioactives, far surpassing the potential health benefits of what is currently available.

A whole food ingredient, as disclosed herein, may be used in formulating foods made by extrusion and/or puffing and/or cooking processes, such as ready to eat breakfast cereals and snack foods. These materials are formulated primarily with cereal grains and may contain flours from one or more cereal grains. The cereal grains utilized, such as corn, wheat, rice, barley, and the like, have a high starch content but relatively little protein. A cereal having more protein content, therefore, may be desirable from a nutritional standpoint. A composition of the present disclosure may contain flour from at least one cereal grain, including corn and or rice, or alternatively, wheat, rye, oats, barley, and mixtures thereof. Cereal grains as used herein may be commercially available, and may be whole grain cereals, but may also be processed from crops according to conventional processes for forming refined cereal grains.

Refined cereal grain, as used herein, may also include derivatives of cereal grains such as starches, modified starches, flours, other derivatives of cereal grains commonly used in the art to form cereals, and any combination of such materials with other cereal grains. A refined corn for example, may be formed from U.S. No. 1 or No. 2 yellow dent corn by dry milling the corn to separate the endosperm from the germ and bran, and forming corn meal, corn grits, or corn flour from the endosperm. Refined wheat grain may be formed according to commercial milling practices from hard or soft wheat varieties, red or white wheat varieties, and may be a wheat flour containing little or no wheat bran, a wheat bran, or a milled wheat product containing flour, bran, and germ (whole wheat flour). Refined rye may be preferably a rye flour which may be formed according to commercial milling practices. Refined rice may be heads, second heads, or brewers rice which may be formed by conventional practices for dehulling rough rice and pearling the dehulled rice, and preferably rough grinding the pearled and dehulled rice into a rice flour. Oats are refined by conventional practices into oatmeal by dehulling and cleaning the oats to form oat groats and milling the oat groats to form oatmeal or oat flour. The refined oats may also be defatted. Barley may be refined according to conventional practices into barley flakes or barley grits by dehulling and cleaning the barley to form clean barley which may be pearled and flaked or ground to form the barley flakes or barley grits.

Breakfast cereal and snack materials may obtain desired flake structures by a process described throughout the disclosure as puffing. Without being bound to a single theory of operation, a food product may be puffed by causing trapped moisture in the flake to change very rapidly from a liquid state to a vapor phase. Rapid heating or a rapid decrease in pressure are commonly used in industry. Gun puffing may be an example of the principle of a rapid decrease in pressure. In this process the cereal flakes are first heated under high pressure and then the pressure may be rapidly released to achieve the puffing effect.

Initial moisture content of an unpuffed material can have a large effect on the puffing of a food product. The specific moisture content that may be best may depend on the particular type of puffing process being utilized. For instance, a moisture content of 12 to 14 percent may be preferred for gun puffing while to 12 percent may be preferred for puffing by a process that rapidly heats the flake. The optimum moisture content for any one puffing technique may routinely be determined experimentally. Additional processing steps may be utilized if it may be so desired. For instance, a toasting operation may be used after the puffing step to change the color of the flake to a more desired rich golden brown. Frequently, a slight toasting step also brings out a pleasant toasted flavor note.

### Baked Goods

Food compositions of the disclosure also include bakery products and baking mixes comprising whole food ingredients according to the disclosure according to known methods. The term “bakery product” includes, but may be not limited to, leavened or unleavened, traditionally flour-based products such as white pan and whole wheat breads (including sponge and dough bread), cakes, pretzels, muffins, donuts, brownies, cookies, pancakes, biscuits, rolls, crackers, pie crusts, pizza crusts, hamburger buns, pita bread, and tortillas. Crostini usually refers to small pieces of toasted or fried bread optionally served with a topping. Typically a crostini has a height (i.e. between the slices) of more than a cracker, ranging from 0.05″ to 2″, from 0.1″ to 1″ and from 0.5″ to 1″. Food products, according to the disclosure, may include a crostini, or similar product, that comprises a whole food ingredient according to the disclosure.

In accordance with embodiments of the disclosure, leavening agents may be included in the dough to produce food products, which involve a rising, such as crackers and breads. Exemplary leavening agents include yeast, baking powder, eggs, and other commercially available leavening agents. Leavening agents may generally comprise less than about 5%, by weight, of the dry ingredients.

Dough, in accordance with embodiments of the disclosure, may also include gums such as xanthum, guar, agar, and other commercially available hydrocolloids typically used for dough binding and conditioning. Additionally, food grade oils may be used to improve sheeting, texture, browning, and taste. Example oils include vegetable oil, soybean oil, sunflower oil, corn oil, and other commercially available oils. Lecithin may also be added to improve emulsification, water binding, and dough release.

In an embodiment, the amount of a whole food ingredient in the bakery products or bakery mixes may be in the range of, at least 0.1 to 20, at least 1 to 10, or at least 2 to 7 grams per 50 gram serving, or approximately 5 or 6 grams per serving. A method of producing a food composition of the disclosure includes forming a cohesive dough by measuring and mixing the dry ingredients using standard mixing equipment.

Bread, rolls, bagels, and English muffins according to the disclosure may have between about 4.8% to about 7% (2.7 g) whole food ingredients of the disclosure per 40 g serving (adding 2 g protein for high protein bread formulation.)

### Bars and Granolas

The present disclosure also includes food compositions such as granola cereals, and bar products, including such as granola bars, nutrition bars, energy bars, sheet and cut bars, extruded bars, baked bars, and combinations thereof. A baked food composition and or bar compositions may be produced according to standard industry recipes, while also substituting in a whole food ingredient of the present disclosure for at least some of the called-for ingredients.

For extruded compositions, protein fortification may be accomplished by supplementing a food bar with edible proteins from at least one high protein content source, as known in the art, and including the whole food ingredient of the present disclosure, either alone or in combination with additional protein. Based upon the weight of the extrudate, or core, a suitable amount of the at least one high protein content source may be about 20% to about 30% by weight. Protein content may be at least about 15% by weight, based upon the weight of the final product.

In the present disclosure, a liquid sweet ingredient, such as corn syrup, for example, high fructose corn syrup, may be used as a carbohydrate content source. In one embodiment, the liquid sweet ingredient provides a moist, chewy texture to the bar, provides sweetness, and serves to distribute the dry ingredients. The liquid sweet ingredient may include, without limitation, corn syrup, high fructose corn syrup, honey, tapioca syrup, among others as known in the art. Additionally, the liquid sweet ingredient, in combination with other binders known in the art, may be useful to bind the other ingredients, such as the protein content and other carbohydrate content sources together. Suitable amounts of the liquid sweet ingredient are about 25% to about 30% by weight, based upon the weight of the extrudate. At least one other carbohydrate content source may be optionally included in the bar of the present disclosure. Examples of suitable carbohydrate content sources for providing a caloric distribution within the above ranges are sugars, such as fructose granules, brown sugar, sucrose, and mixtures thereof, and cereal grains such as rice, oats, corn, and mixtures thereof. A snack may contain at least one sugar and at least one carbohydrate. Based upon the weight of the core, suitable amounts of these ingredients are about 0.1% to about 10% by weight of at least one sugar, and about 12% to about 18% by weight of at least one cereal grain.

A bar may also optionally comprise a fat. Suitable sources of fats include those known in the art to be suitable for bar-type products and include milk, chocolate, and coconut oils, creams, and butters; nut butters such as peanut butter, and an oil such as vegetable oil. Also, a liquid wetting agent may be present in the composition, to facilitate mixing and binding of the dry ingredients to enhance moistness and chewiness of the snack. Exemplary of such wetting agents are molasses, honey, and vegetable oils, and mixtures thereof. A suitable amount of the at least one wetting agent may be about 2% to 5% by weight. Suitable amounts of the flavoring ingredients range up to about 3% by weight. Also it is known in the art that carbohydrate content sources, useful in the present disclosure, may also be substantial sources of proteins and/or fats. For example, peanut flour, oats, and wheat germ each provide substantial amounts of proteins, carbohydrates, and fats. Dietary fiber may be included in the bar. Suitable amounts are about 1% to about 8%, or about 5% by weight fiber, based upon the weight of the final product. Suitable sources of dietary fiber are rolled oats and brans. The bar may be topped with conventional toppings, such as granola, crushed nuts, and the like, to enhance flavor and visual appeal. Suitable topping amounts are about 2% to 3% by weight of the final product.

In some embodiments, the whole food ingredient produced from a process as described herein is manufactured in a bar at high levels of fat. In some embodiments, the whole food ingredient produced from a process as described herein is manufactured in a bar at high levels of fat so as to not induce any phase separation. In some embodiments, the bar includes a fat content of about 20%. In some embodiments, the bar includes a fat content of about 25%. In some embodiments, the bar includes a fat content of about 30%. In some embodiments, the bar does not include an emulsifier.

In one embodiment, the nutritional snacks of the present disclosure are made by first mixing the liquid ingredients and the optional wetting agent. Next, the minor dry components are added to the mixed liquids. The minor dry components include ingredients such as, for example, minerals and vitamins, optionally premixed, and optionally including salt. The major dry ingredients may then admixed with the mixed liquids and minor dry ingredients to form a substantially homogeneous mixture. The major dry ingredients include e.g., sugars and cereal grains. The major dry ingredients also include the high protein content sources including the whole food ingredient of the disclosure. The flavoring ingredients, such as cocoa or coconut, may be added with the minor dry ingredients or with the major dry ingredients. All mixing may be in the same mixer or blender. Suitable mixing and blending equipment may include conventional vertical and horizontal type mixers and blenders.

Mixed ingredients may be transferred via conveyor belts and hoppers, for example, to a conventional bar extruder, having opposing rollers which force the mixture through a die to form the extrudate or core. The extrusion may be performed at about room temperature. Cooking or heating after extrusion may be usually unnecessary. An extruded shape may be a rectangular bar, but other shaped bars, known in the snack bar art, such as cylindrical, and semicylindrical shaped bars may be made using appropriate extruder dies.

In accordance with the present disclosure, the granola cereals and bar products, the dry ingredients may include a food particulate. A food particulate may include, without limitation, any edible food particulate. Such particulates may include flours, meals, cereal grains, cereal flakes, crisped rice, puffed rice, oats, crisped oats, granola, wheat cereals, protein nuggets, textured soy flour, textured soy protein concentrate, texturized protein ingredients such as those disclosed herein, flavored nuggets, cookie pieces, cracker pieces, pretzel pieces, crisps, soy grits, nuts, fruit pieces, vegetable pieces, corn cereals, seeds, popcorn, yogurt pieces, and combinations of any thereof.

For example, for grain-based bars, an appropriate amount of whole food ingredients includes between about 20% to about 33.3% (20 g) whole food ingredients per 60 g serving (with for example, 15 g protein in a high protein bar). Where the bar contains a fruit and/or vegetable, an appropriate amount of whole food ingredient may include about 20% (8 g) whole food ingredients per 45 g serving (adding 6 g to a total of 8 g of fiber in a high protein type bar.)

After extrusion, the product may be dried. The final product may have a moisture content of from, about 0.1% to about 30%, about 1% to about 15%, or about 1% to about 8%, depending on the desired characteristics of the finished product. In one embodiment, an extruded nutritional protein bar may include 21.33 g/60 g of whole food ingredients of the present disclosure, with the balance including carbohydrate, nuts, oils, with proportions determined by conventional processes known in the art.

### Liquids

Food compositions of the present disclosure also include functional drinks, nutritional beverages (protein, energy, and meal replacement), smoothies and smoothie bases, juices, soups and soup bases, fats and oils. For example, salad dressings may include about 8 g whole food ingredients of the disclosure per 30 g serving; a fruit juice, fruit flavored drink, fruit nectar may include about 1% by weight of whole food ingredient product of the disclosure. A vegetable juice such as a tomato juice may include between about 2.5% to about 20% (8 g) whole food ingredient of the disclosure per 240 mL serving. A smoothie may contain between about 3.5% to 20% by weight or between 9 and 20 g of whole food ingredient product of the disclosure, for example, about 40 g per 450 mL serving.

For a soup or soup base (mix), prepared soups, dry soup mixes, and condensed soups, a whole food ingredient may be added in an amount of between 0.96% to 3.3% by weight (8 g) per 242 g serving. For a confectionary, such as a chocolate dessert (peanut butter cup), a food product of the disclosure may, for example, include about 2.67 g per 40 g serving.

### Dairy Products

In one embodiment, a food composition may include an alternative dairy product comprising a whole food ingredient, according to the disclosure. An alternative dairy product, according to the disclosure, includes, without limitation, products such as faux skimmed milk, faux whole milk, faux cream, faux fermented milk product, faux cheese, faux yogurt, faux butter, faux dairy spread, faux butter milk, faux acidified milk drink, faux sour cream, faux ice cream, faux flavored milk drink, or a faux dessert product based on milk components such as custard. Methods for producing alternative dairy products using alternative proteins, such as plant-based proteins as disclosed herein, including nuts (almond, cashew), seeds (hemp), legumes (pea), rice, and soy are known in the art. These known methods for producing alternative dairy products using a plant-based protein may be adapted to use with a whole food ingredient.

An alternative dairy product, according to the disclosure, may additionally comprise non-milk components, such as oil, protein, carbohydrates, and mixtures thereof. Dairy products may also comprise further additives such as enzymes, flavoring agents, microbial cultures, salts, thickeners, sweeteners, sugars, acids, fruit, fruit juices, any other component known in the art as a component of, or additive to a dairy product, and mixtures thereof.

A whole food ingredient, according to the disclosure, may be used to create a whole food ingredient-based “milk” beverage produced by using the whole food ingredient, optionally, by combining the product as a powder with oils and carbohydrates to form an emulsion. Methods for creating vegan protein milks using soybeans as the protein source are known in the art and protein source may simply be substituted with whole food ingredient protein. As a non-limiting example, a typical unsweetened “milk” drink includes, per 243 ml serving, a total of 4 g carbohydrates which may include 1 g of sugar, 4 g of fat or oil from any source, and whole food ingredient solids sufficient to provide between about 1-10 g of protein, the drink being in the form of a stable emulsion of oil, water, and protein. The ratio of whole food ingredient to the other ingredients may be varied depending on the desired protein level of the drink and the desired organoleptic properties. Typically, the amount will vary between about 0.1-10% g protein per mL beverage, or about 0.5 to 7%, 1% to 5% or about 1.1-1.3%. The resulting slurry or purde may optionally be brought to a boil in order to e.g., improve its flavor, and to sterilize the product. Heating at or near the boiling point may be continued for a period of time, 15-20 minutes, followed by optional removal of insoluble residues by e.g., filtration.

A whole food ingredient, according to the disclosure, may be used to create a whole food ingredient-based “yogurt” beverage produced by using whole food ingredients, optionally, by combining whole food ingredients with the other ingredients in powder form. Methods for creating vegan yogurt using soybeans as the protein source are known in the art, and protein sources may simply be substituted with whole food ingredient protein, for example, to create the yogurts of the disclosure. For example, a whole food ingredient may be used as 1 to about 10%

Whole food ingredient solids sufficient to provide between about 1-10 g of protein per serving. Other ingredients in the yogurt may include, without limitation, as known in the art, nut milks (almond, cashew, for example), fats or oils (such as coconut cream, coconut oils), sugar, and thickening or gelling agents including, without limitation, agents such as locust bean gums, pectin, and the like. The composition, in some embodiments, will contain no less than 2.5% fat from a plant source, such as, without limitation, almond, cashew, and/or coconut, and no less than 3.5% protein. Frozen yogurts may also be prepared and may have similar compositions.

A whole food ingredient may be used to create a whole food ingredient-based “ice cream” beverage produced by using whole food ingredients, optionally, by combining whole food ingredients with the other ingredients in powdered form. Methods for creating vegan ice cream using soybeans as the protein source are known in the art, and protein sources may simply be substituted with whole food ingredient protein, for example, to create the ice creams of the disclosure. For example, whole food ingredients may be used as 1.1% to 7% (10.7 g) whole food ingredient solids sufficient to provide between about 1-10 g of protein per serving. Other ingredients in the ice cream may include, without limitation, as known in the art, creams, fats or oils (such as coconut cream, coconut oil), sugar, and thickening or gelling agents, including, without limitation, agents such as locust bean gum, pectin, emulsifiers such as lecithin, and the like. The composition, in some embodiments, will contain no less than 10% fat from a plant source, such as, without limitation, almond, cashew, and/or coconut, and no less than 3.5% protein and no less than 35% total solids.

The present disclosure also includes beverages and beverage bases comprising a whole food ingredient which may be used as non-dairy-based meal replacement beverages. A whole food ingredient, according to the disclosure, may be used to prepare a meal replacement beverage that may optionally be non-dairy-based. Methods for creating vegan meal replacement beverages using soybeans as the protein source are known in the art, and a protein source may be substituted with whole food ingredient protein of the disclosure. For example, a typical meal replacement drink might include, per 243 ml serving, a total of 4 g carbohydrates which may include 1 g of sugar, 4 g of fat or oil from any source, and whole food ingredient solids sufficient to provide between about 2-30 g of protein. The ratio of whole food ingredients may be varied depending on the desired protein level of the drink and the desired organoleptic properties. Typically, the amount will vary between about 0.1-15% g protein per mL beverage, or about 0.5 to 7%, 1% to 5% or about 1.1-1.3%. A resulting slurry or purde may optionally be brought to a boil in order to e.g., improve its flavor, and sterilize the product. Heating at or near the boiling point may be continued for a period of time, 15-20 minutes, followed by optional removal of insoluble residues by e.g., filtration. A ready-to-mix beverage powder may include 32.7 g of whole food ingredient per 35 g serving. Examples of products include protein shakes and smoothies, and dietary and nutritional beverages, including meal replacement beverages and smoothies.

### Reaction Flavors

In one embodiment, the present disclosure includes a whole food ingredient effective at masking, ameliorating, or otherwise changing unappealing flavors in a food product. Seasonings, vitamins, minerals, and/or preservatives may be added before or after the extruding and/or cooking and/or puffing steps. Edible oils and/or fats may also be added; or emulsifiers, sweeteners, and combinations thereof.

The Maillard reaction is a chemical reaction between amino acids and reducing sugars that can give browned food its distinctive flavor. Seared steaks, pan-fried dumplings, cookies, and other kinds of biscuits, breads, toasted marshmallows, and many other foods undergo this reaction. The reaction is a form of non-enzymatic browning, which typically proceeds rapidly from around 140 to 165° C. (280 to 330° F.). Many recipes call for an oven temperature high enough to ensure that a Maillard reaction occurs. At higher temperatures, caramelization and, subsequently pyrolysis may become more pronounced. In a Maillard reaction, the reactive carbonyl group of the sugar reacts with the nucleophilic amino group of the amino acid and forms a complex mixture of poorly characterized molecules responsible for a range of aromas and flavors. This process may be accelerated in an alkaline environment (e.g., lye applied to darken pretzels; see lye roll), as the amino groups (RNH3+→RNH2) are deprotonated, and therefore have an increased nucleophilicity.

In one embodiment, the present disclosure includes a method to prepare a reaction flavor composition. In some embodiments, the whole food ingredient is a reaction flavor composition. In some embodiments, the whole food ingredient does not require a reaction to impart flavor characteristics. In some embodiments, an edible material comprises providing at least one flavor component capable of facilitating Maillard and/or Strecker reactions. In another embodiment, the method includes mixing the whole food ingredient and the reaction flavor component. In yet another step, the method includes processing the mixture to form the reaction flavor composition.

### Artificial Meat

In an embodiment, the present disclosure also includes a method to prepare a textured plant-based protein product useful for products such as meat-structured meat analogs or meat extenders. In one embodiment, this textured plant-based meat analog or meat extender has a texture associated with meat. The method optionally provides a “meat structured protein product,” which may be made from the “texturized protein product” as known in the art. Of high importance to a meat-structured protein, product may be a texturized protein product which refers to a product comprising protein fiber networks and/or aligned protein fibers that produce meat-like textures. It may be obtained from a dough after application of e.g., mechanical energy (e.g., spinning, agitating, shaking, shearing, pressure, turbulence, impingement, confluence, beating, friction, wave), radiation energy (e.g., microwave, electromagnetic), thermal energy (e.g., heating, steam texturizing), enzymatic activity (e.g., transglutaminase activity), chemical reagents (e.g., pH adjusting agents, kosmotropic salts, chaotropic salts, gypsum, surfactants, emulsifiers, fatty acids, amino acids), other methods that lead to protein denaturation and protein fiber alignment, or combinations of these methods, followed by fixation of the fibrous and/or aligned structure (e.g., by rapid temperature and/or pressure change, rapid dehydration, chemical fixation, redox), and optional post-processing after the fibrous and/or aligned structure may be generated and fixed (e.g., hydrating, marinating, drying, coloring). Methods for determining the degree of protein fiber network formation and/or protein fiber alignment are known in the art. In some embodiments, at least about 55%, at least about 65%, at least about 75%, at least about 85%, or at least about 95% of the protein fibers are substantially aligned. Protein fiber networks and/or protein fiber alignments may impart cohesion and firmness, whereas open spaces in the protein fiber networks and/or protein fiber alignments may tenderize the meat-structured protein products and provide pockets for capturing water, carbohydrates, salts, lipids, flavorings, and other materials that are slowly released during chewing to lubricate the shearing process and to impart other meat-like sensory characteristics.

In one embodiment, the method to make a textured plant-based protein product includes the step of providing a whole food ingredient according to the present disclosure. Further, the food product, according to the present disclosure, has reduced undesirable flavor and/or reduced undesirable aroma compared with a food product without the whole food ingredient, as described herein. A method of making may include providing an additional material, such as an additional high-protein material, fiber, starch, or other materials; and mixing whole food ingredients and the additional material to form a mixture; optionally preconditioning the mixture, e.g., roasting the whole food ingredient, or adding steam and/or water to the mixture, and extruding the mixture under heat and pressure under conditions capable of forming a textured plant-based protein product useful for products such as meat-structured meat analogs or meat extenders that contain no animal products. The method to prepare a textured plant-based protein product may also include the step of providing an optional carbohydrate component. The carbohydrate ingredients may be classified as a starch, a flour, or an edible fiber, and the carbohydrate component may comprise one or more types of starch, flour, edible fiber, and combinations thereof.

Starch may be a primary carbohydrate source used to help the formation of the product texture in textured plant-based protein products. Typical starches used include rice starch, wheat starch, oat starch, corn starch, potato starch, cassava starch, and tapioca starch, although starch from any source may be contemplated. Overall, the swelling ability of starch, solubility, amount of amylose leaching out during gelatinization, and the ability to produce a viscous paste, have an effect on the textured plant-based protein product. Chemical alterations occur due to structural changes of the macromolecules in the feed blend, such as starch gelatinization and protein denaturation, as well as the incorporation of water into the molecular matrix, all of which convert the raw feed particles into a viscoelastic dough under a pressurized environment. Physical changes, on the other hand, are related to product expansion due to a drastic pressure drop and water evaporation during die exit. In one embodiment, the textured plant-based protein product includes an edible fiber, including but not limited to the whole food ingredient of the present disclosure. Examples of suitable edible fiber include but are not limited to bamboo fiber, barley bran, carrot fiber, citrus fiber, corn bran, soluble dietary fiber, insoluble dietary fiber, oat bran, pea fiber, soy fiber, soy polysaccharide, wheat bran, wood pulp cellulose, modified cellulose, seed husks, oat hulls, citrus fiber, carrot fiber, corn bran, soy polysaccharide, barley bran, and rice bran. The fiber may be present in the dry pre-mix from about 0.1% to about 10% by weight.

### Bioactive Content

In some embodiments, the food product includes one or more bioactives. In some embodiments, the food product includes 0.25, 0.5, 1, 1.5, 2.0, 2.5, 3.0, 3.5, 4.0, 4.5, 5.0, 7.5, 10, 12.5, 15, 17.5, 20, 22.5, 25, 27.5, 30, 32.5, 35, 37.5, 40, 42.5, 45, 47.5, 50%, 55%, 60% w/w of one or more bioactives, or ranges including, between, and/or spanning the aforementioned values. In some embodiments, the food product includes about 0.5% to about 5% w/w of one or more bioactives. In some embodiments, the food product includes about 0.5% to about 10% w/w of one or more bioactives. In some embodiments, the food product includes about 1% w/w to about 10% of one or more bioactives. In some embodiments, the food product includes about 3.0% to about 20% w/w one or more bioactives. In some embodiments, the food product includes about 5.0% to about 15% w/w of one or more bioactives. In some embodiments, the food product includes about 8.0% to about 15% w/w of one or more bioactives. In some embodiments, the food product includes about 10% to about 20% w/w of one or more bioactives. In some embodiments, the food product includes about 15% w/w to about 25% of one or more bioactives. In some embodiments, the food product includes about 20% to about 25% w/w of one or more bioactives. In some embodiments, the food product includes about 25% w/w one or more bioactives. In some embodiments, the food product includes about 30% to about 40% w/w of one or more bioactives. In some embodiments, the food product includes about 35 to about 50% w/w of one or more bioactives. In some embodiments, the food product includes about 40% to about 50% w/w one or more bioactives. In some embodiments, the food product includes about 45% to about 50% w/w of one or more bioactives. In some embodiments, the food product includes about 50% w/w one or more bioactives. In some embodiments, the food product includes about 55% w/w one or more bioactives. In some embodiments, the food product includes about 60% w/w one or more bioactives.

In some embodiments, the food product retains one or more bioactives from an extrusion process described herein. In some embodiments, the food product retains 1, 10, 20, 30, 40, 50, 60, 70, 80, 90, 100, 150, 200, 250, 300, 350, 400, 450, 500, 550, 600, 650, 700, 750, 800, 850, 900, 950, 1000, 2000 mg/Kg of the one or more bioactives, or ranges including, between, and/or spanning the aforementioned values.

In some embodiments, the one more or more bioactives is a tyramine containing hydroxycinnamic acid amide. In some embodiments, the tyramine containing hydroxycinnamic acid amide includes, in a non-limiting example, N-trans-caffeoyltyramine, N-cis-caffeoyltyramine, N-trans-feruloyltyramine, N-cis-feruloyltyramine, p-coumaroyltyramine, or a combination thereof. In some embodiments, the food product includes about 0.01% to about 20% w/w of N-trans-caffeoyltyramine. In some embodiments, the food product includes about 0.01% to about 20% w/w of N-trans-feruloyltyramine.

In some embodiments, the food product, whole food ingredient, whole food powder, extract ingredient, pressed cake, liquid ingredient, or formulation as described herein is administered at a dose in the range of about 1-2000 mg/kg body weight. In some embodiments, the food product or formulation as described herein is administered at a dose in the range of about 1-10, 1-20, 1-30, 1-40, 1-50, 1-60, 1-70, 1-80, 1-90, 1-100, 1-200, 1-300, 1-400, 1-500, 1-600, 1-700, 1-800, 1-900, 1-1000, 1-11, 1-12, 1-13, 1-13, 1-14, 1-15, 1-16, 1-17, 1-18, 1-19, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 20-30, 20-40, 20-50, 20-60, 20-70, 20-80, 20-90, 20-100, 20-200, 20-300, 20-400, 20-500, 20-600, 20-700, 20-800, 20-900, 20-1000, 30-40, 30-50, 30-60, 30-70, 30-80, 30-90, 30-100, 30-200, 30-300, 30-400, 30-500, 30-600, 30-700, 30-800, 30-900, 30-1000, 40-50, 40-60, 40-70, 40-80, 40-90, 40-100, 40-200, 40-300, 40-400, 40-500, 40-600, 40-700, 40-800, 40-900, 40-1000, 50-60, 50-70, 50-80, 50-90, 50-100, 50-200, 50-300, 50-400, 50-500, 50-600, 50-700, 50-800, 50-900, 60-70, 60-80, 60-90, 60-100, 60-200, 60-300, 60-400, 60-500, 60-600, 60-700, 60-800, 60-900, 60-1000, 70-80, 70-90, 70-100, 70-200, 70-300, 70-400, 70-500, 70-600, 70-700, 70-800, 70-900, 70-1000, 80-90, 80-100, 80-200, 80-300, 80-400, 80-500, 80-600, 80-700, 80-800, 80-900, 80-100, 90-100, 90-200, 90-300, 90-400, 90-500, 90-600, 90-700, 90-800, 90-900, 90-1000, 100-150, 100-200, 100-300, 100-400, 100-500, 100-600, 100-700, 100-800, 100-900, or 100-1000 mg/kg of body weight. In some embodiments, the the food product, whole food ingredient, whole food powder, extract ingredient, pressed cake, liquid ingredient, or formulation as described herein is administered at a dose of about 20, 30, 50, 55, 60, 65, 70, 80, 90, 95, 100, 150, 200, 250, 500, 750, 1000, 1250, 1500, 1750, 2000 mg/kg of the body weight. In some embodiments, the food product, whole food ingredient, whole food powder, extract ingredient, pressed cake, liquid ingredient, or formulation as described herein is administered at a dose less than about 1, 5, 10, 15, 20, 25, 30, 35, 40, 50, 75, 100 mg/m2 of the body surface area. In some embodiments, the food product, whole food ingredient, whole food powder, extract ingredient, pressed cake, liquid ingredient, or formulation as described herein is administered at a dose greater than about 1, 2, 3, 4, 5, 6, 7, 8, 9, 10, 15, 20, 25, 30, 40, 50, 55, 60, 65, 70, 75, 80, 85, 90, 95, or 100 g/kg of a subjects body weight.

In some embodiments, the food product, whole food ingredient, whole food powder, extract ingredient, pressed cake, liquid ingredient, or formulation comprises total tyramine containing hydroxycinnamic acid amide from about 0.1 mg-10 mg, 0.1 mg-25 mg, 0.1 mg-30 mg, 0.1 mg-50 mg, 0.1 mg-75 mg, 0.1 mg-100 mg, 0.5 mg-10 mg, 0.5 mg-25 mg, 0.5 mg-30 mg, 0.5 mg-50 mg, 0.5 mg-75 mg, 0.5 mg-100 mg, 1 mg-10 mg, 1 mg-25 mg, 1 mg-30 mg, 1 mg-50 mg, 1 mg-75 mg, 1 mg-100 mg, 2 mg-10 mg, 2 mg-25 mg, 2 mg-30 mg, 2 mg-50 mg, 2 mg-75 mg, 2 mg-100 mg, 3 mg-10 mg, 3 mg-25 mg, 3 mg-30 mg, 3 mg-50 mg, 3 mg-75 mg, 3 mg-100 mg, 4 mg-100 mg, 5 mg-10 mg, 5 mg-25 mg, 5 mg-30 mg, 5 mg-50 mg, 5 mg-75 mg, 5 mg-300 mg, 5 mg-200 mg, 7.5 mg-15 mg, 7.5 mg-25 mg, 7.5 mg-30 mg, 7.5 mg-50 mg, 7.5 mg-75 mg, 7.5 mg-100 mg, 7.5 mg-200 mg, 10 mg-20 mg, 10 mg-25 mg, 10 mg-50 mg, 10 mg-75 mg, 10 mg-100 mg, 15 mg-30 mg, 15 mg-50 mg, 15 mg-100 mg, 20 mg-20 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, the food product, whole food ingredient, whole food powder, extract ingredient, pressed cake, liquid ingredient, or formulation comprises total n-trans-caffeoyltyramine/n-trans-feruloyltyramine from about 20 mg-60 mg, 27 mg-60 mg, 20 mg-45 mg, or 27 mg-45 mg. In some embodiments, the food product, whole food ingredient, whole food powder, extract ingredient, pressed cake, liquid ingredient, or formulation comprises total n-trans-caffeoyltyramine/n-trans-feruloyltyramine from about 1 mg-5 mg, 1 mg-7.5 mg, 2.5 mg-5 mg, 2.5 mg-7.5 mg, 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the food product, whole food ingredient, whole food powder, extract ingredient, pressed cake, liquid ingredient, or formulation comprises total n-trans-caffeoyltyramine/n-trans-feruloyltyramine as described herein is greater than, equal to, or about 0.1 mg, 0.3 mg, 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 300 mg, about 400 mg, about 500 mg, about 600 mg, about 700 mg, about 800 mg, about 900 mg, or about 1000 mg or ranges including, between, and/or spanning the aforementioned values. In some embodiments, the food product, whole food ingredient, whole food powder, extract ingredient, pressed cake, liquid ingredient, or formulation comprises total n-trans-caffeoyltyramine/n-trans-feruloyltyramine as described herein dose is about less than about 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg or ranges including, between, and/or spanning the aforementioned values.

In some embodiments, the food product, whole food ingredient, whole food powder, extract ingredient, pressed cake, liquid ingredient, or formulation comprises n-trans-caffeoyltyramine from about 1 mg-5 mg, 1 mg-7.5 mg, 2.5 mg-5 mg, 2.5 mg-7.5 mg, 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the food product, whole food ingredient, whole food powder, extract ingredient, pressed cake, liquid ingredient, or formulation comprises n-trans-caffeoyltyramine greater than, equal to, or about 0.1 mg, 0.3 mg, 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 300 mg, about 400 mg, about 500 mg, about 600 mg, about 700 mg, about 800 mg, about 900 mg, about 1000 mg or ranges including, between, and/or spanning the aforementioned values. In some embodiments, the food product, whole food ingredient, whole food powder, extract ingredient, pressed cake, liquid ingredient, or formulation comprises n-trans-caffeoyltyramine as described herein dose is about less than about 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg or ranges including, between, and/or spanning the aforementioned values.

In some embodiments, the food product, whole food ingredient, whole food powder, extract ingredient, pressed cake, liquid ingredient, or formulation comprises n-trans-feruloyltyramine from about 1 mg-5 mg, 1 mg-7.5 mg, 2.5 mg-5 mg, 2.5 mg-7.5 mg, 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the food product, whole food ingredient, whole food powder, extract ingredient, pressed cake, liquid ingredient, or formulation comprises n-trans-feruloyltyramine greater than, equal to, or about 0.1 mg, 0.3 mg, 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 300 mg, about 400 mg, about 500 mg, about 600 mg, about 700 mg, about 800 mg, about 900 mg, about 1000 mg or ranges including, between, and/or spanning the aforementioned values. In some embodiments, the food product, whole food ingredient, whole food powder, extract ingredient, pressed cake, liquid ingredient, or formulation comprises n-trans-feruloyltyramine as described herein dose is about less than about 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg or ranges including, between, and/or spanning the aforementioned values.

In some embodiments, the food product retains the natural micronutrients from an extrusion process described herein. In some embodiments, the food product retains 1, 10, 20, 30, 40, 50, 60, 70, 80, 90, 100, 150, 200, 250, 300, 350, 400, 450, 500, 550, 600, 650, 700, 750, 800, 850, 900, 950, 1000, 2000 mg/Kg of the micronutrients, or ranges including, between, and/or spanning the aforementioned values. In some embodiments, the food product further includes the addition of one or more micronutrients. In some embodiments, the one or more micronutrients include manganese, vitamin A, vitamin B, vitamin C, vitamin K, copper, fiber, iron, chromium, calcium, protein, carbohydrates, fat, fiber, sugars, or starch.

In some embodiments, the food product includes about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25% w/w moisture content, or ranges including, between, and/or spanning the aforementioned values.

In some embodiments, the food product of this disclosure may further comprise one or more acceptable excipients and/or carriers for oral consumption. Suitable excipients and/or carriers include maltodextrin, calcium carbonate, dicalcium phosphate, tricalcium phosphate, microcrystalline cellulose, dextrose, rice flour, magnesium stearate, stearic acid, croscarmellose sodium, phospholipid, sodium starch glycolate, crospovidone, sucrose, vegetable gums, lactose, methylcellulose, povidone, carboxymethylcellulose, corn starch, cyclodextrins, chitosan and its derivatives and the like (including mixtures thereof). In some embodiments, the carrier includes at least one of calcium carbonate, magnesium stearate, maltodextrin, and mixtures thereof. The various ingredients and the excipient and/or carrier are mixed and formed into the desired form using conventional techniques.

In some embodiments, the food product may further comprise a nutraceutically acceptable excipient or nutraceutically acceptable carrier. In some embodiments, the nutraceutically acceptable excipient or nutraceutically acceptable carrier is selected from medium chain triglycerides, diglycerides, and monoglycerides, caprylic acid, linoleic acid, linoleic acid, oleic acid, keto-oleic acid. In some embodiments, the nutraceutically acceptable excipient or nutraceutically acceptable carrier further includes a second fatty acid component having one or more non-activated fatty acids selected from linoleic acid, α-linoleic acid, γ-linoleic acid, oleic acid, eicosapentaenoic acid (EPA), docosahexaenoic acid (DEIA), or derivatives thereof.

In some embodiments, the food product may further include one or more secondary agents selected from vitamin A, vitamin B, vitamin B-1, vitamin B-2, vitamin B-6, vitamin B-12, vitamin C, vitamin D, vitamin D3, vitamin E, selenium, β-carotene, Ginkgo biloba, goldenseal, valerian, ginseng, echinacea, grape seed extracts, ephedra, yucca concentrates, green tea extract, rice bran extract, wheat germ, wheat germ extract, beeswax, red yeast rice extract, stevia leaf extract, flaxseed oil, borage seed oil, coenzyme Q10, glucosamine derivatives, methylsulfonylmethane, pantothenic acid, biotin, thiamin, riboflavin, niacin, folic acid, palmitic acid, and derivatives thereof. In some embodiments, the food product may include one or more secondary agents selected from policosanols, phospholipid, guggulipids, rice bran extract, wheat germ, wheat germ extract, beeswax, and red yeast rice extract, and such the food product may be formulated to promote a healthy heart and circulatory system. In other embodiments, the food product may include one or more secondary agents selected from vitamin B-1, vitamin B-2, vitamin B-6, vitamin B-12, vitamin C, vitamin D, vitamin D3, vitamin E, selenium, goldenseal, valerian, ginseng, and echinacea and such the food product may be formulated to promote healthy cell proliferation. In still other embodiments, the food product may include one or more secondary agents selected from vitamin A, vitamin C, vitamin E, and n-carotene, and such the food product may be formulated to promote healthy eyes. In yet other embodiments, the food product may include one or more secondary agents selected from vitamin A, vitamin C, vitamin E, selenium, Ginkgo biloba, goldenseal, valerian, ginseng, echinacea, ephedra, green tea extract, and yucca concentrate. In some embodiments, the food product may further include a dietary supplement. In some embodiments, the food product may be formulated to promote metabolic health. In some embodiments, the food product may be formulated to provide increased metabolic nutrients.

In some embodiments, the food product may further comprise at least one or more secondary agents, which is a phytonutrient (e.g., isoflavonoids, oligomeric proanthcyanidins, indol-3-carbinol, sulforaphone, fibrous ligands, plant phytosterols, ferulic acid, anthocyanocides, triterpenes, omega 3/6 fatty acids, conjugated fatty acids such as conjugated linoleic acid and conjugated linolenic acid, polyacetylene, quinones, terpenes, catechins, gallates, and quercetin). Sources of plant phytonutrients include, but are not limited to, lecithin, isoflavones, brown rice germ, royal jelly, bee propolis, acerola berry juice powder, Japanese green tea, grape seed extract, grape skin extract, carrot juice, bilberry, flaxseed meal, bee pollen, Ginkgo biloba, primrose (evening primrose oil), red clover, burdock root, dandelion, parsley, rose hips, milk thistle, ginger, Siberian ginseng, rosemary, curcumin, garlic, lycopene, grapefruit seed extract, spinach, and broccoli.

In some embodiments, the food product may further comprise an antioxidant. As used herein, the term “antioxidant” refers broadly to a molecule that inhibits the oxidation of other molecules. In some embodiments, the antioxidant is present in an amount of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg or greater, or a value within a range defined by any two of the aforementioned values.

In some embodiments, the food product may further comprise a stimulant. As used herein, the term “stimulant” refers to one or more compounds that temporarily increase the rate of body functions. Stimulants can include, for example, amineptine, amiphenazole, amphetamines, bromantan, caffeine, carphedon, ephedrines, fencamfamine, mesocarb, pentylentetrazol, pipradol, salbutamol, salmeterol, terbutaline, and related substances. In some embodiments, the stimulant is present in an amount of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg or greater, or a value within a range defined by any two of the aforementioned values.

In some embodiments, the food product may further comprise a lipid. As used herein, the term “lipid” refers to fatty acids and derivatives thereof, as well as substances related biosynthetically or functionally to such compounds. In some embodiments, the lipid is present in an amount of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg or greater, or a value within a range defined by any two of the aforementioned values.

In some embodiments, the food product further includes omega 3 fatty acids. Omega 3 fatty acids can be prepared in powder formulations and can include, for example, eicosapentaenoic acid, docosahexaenoic acid, linolenic acid, hexadecatrienoic acid, stearidonic acid, eicosatrienoic acid, eicosatetraenoic acid, heneicosapentaenoic acid, docosapentaenoic acid, tetracosapentaenoic acid, or tetracosahexaenoic acid, or analogs or derivatives thereof, or combinations thereof. In some embodiments, the omega 3 fatty acid is present in an amount of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg or greater, or a value within a range defined by any two of the aforementioned values.

In some embodiments, the food product further includes one or more pH control agents.

In some embodiments, the one or more pH control agents are selected from the group consisting of Acetic acid, Adipic acid, Citric acid, Fumaric acid, Glucono-δ-lactone, Gluconic acid, Lactic acid, Malic acid, Maleic acid, Tartaric acid, Succinic acid, Propionic acid, Ascorbic acid, Phosphoric acid, Sodium orthophosphate, Potassium orthophosphate, Calcium orthophosphate, Sodium diphosphate, Potassium diphosphate, Calcium diphosphate, Pentasodium triphosphate, Pentapotassium triphosphate, Sodium polyphosphate, Potassium polyphosphate, Carbonic acid, Sodium carbonate, Sodium bicarbonate, Potassium carbonate, Calcium carbonate, Magnesium carbonate, Magnesium oxide, or any combination thereof.

In some embodiments, the food product may further comprise a flavoring agent.

In some embodiments, the flavoring agent may be a flavor selected from berry, strawberry, chocolate, cocoa, lemon, butter, almond, cashew, macadamia nut, coconut, blueberry, blackberry, raspberry, peach, lemon, lime, mint, orange, banana, chili pepper, cinnamon, and pineapple.

In some embodiments, the food product may further comprise one or more coating layers. In some embodiments, at least one of the one or more coating layers may be an enteric coating, and in still further embodiments, the core may further include one or more agents selected from solubilizers, stabilizers, colorants, plastizers diluents, fillers, disintegrants, binders, lubricants, surfactants, hydrophobic vehicles, water soluble vehicles, emulsifiers, buffers, humectants, moisturizers, antioxidants, or preservatives. In some embodiments, the core, at least one of the one or more coating layers, or a combination thereof further includes one or more secondary agents.

In some embodiments, the food product further includes one or more alkaloids. In some embodiments, the one or more alkaloid is selected from piperine, chavicine, isochavicine, isopiperine, capsaicin, vanillyl alcohol, vanillin, vanillic acid, acetovanillon, vanillylmandelic acid, and mohovanillic acid.

An aspect of the disclosure includes a food product can be combined with a carrier and provided in any suitable form for consumption by or administration to a subject. In some embodiments, the food product is black pepper. In some embodiments, the food product is a white peppercorn. In some embodiments, the food product is green peppercorn. Suitable consumable forms include, but are not limited to, a dietary supplement, food ingredient or additive, a medical food, or nutraceutical.

A food ingredient or additive is an edible substance intended to result, directly or indirectly, in its becoming a component or otherwise affecting the characteristic of any food (including any substance intended for use in producing, manufacturing, packing, processing, preparing, treating, packaging, transporting, or holding food). A food product, in particular a functional food, is a food fortified or enriched during processing to include additional complementary nutrients and/or health-beneficial ingredients. A food product according to this disclosure can, e.g., be in the form of butter, margarine, sweet or savory spreads, condiment, biscuits, nutrition bars, energy bars and cereal bars, bread, cake, cereal, candy, confectionery, soup, milk, yogurt or a fermented milk product, grains, cheese, juice-based, fruit based and vegetable-based beverages, fermented beverages, shakes, flavored waters, teas, coffees, oil, prepared meals, dairy product analogs, eggs and egg product analogs, soups and soup mixes, or any other suitable food.

A dietary supplement is a product taken by mouth that contains a compound of the disclosure and is intended to supplement the diet. A nutraceutical is a product derived from a food source that provides extra health benefits, in addition to the basic nutritional value found in the food. Dietary supplements and nutraceuticals can be found in many forms such as tablets, coated tablets, pills, capsules, pellets, granules, softgels, gelcaps, liquids, powders, emulsions, suspensions, elixirs, syrup, and any other form suitable for use.

In some embodiments, a food product may include a carrier and a preservative to reduce or retard microbial growth. In some embodiments, the preservative is about 5% w/w of the food product. In some embodiments, the preservative is about 0.5%, about 1.0%, about 1.5%, about 2.0%, about 2.5%, about 3.0%, about 3.5%, about 4.0%, about 4.5%, about 5.0% w/w of the food product, or ranges including, between, and/or spanning the aforementioned values.

Non-limiting examples of preservatives include sodium benzoate, methyl parabens, propyl parabens, sodium nitrite, sulphur dioxide, sodium sorbate and potassium sorbate. Other suitable preservatives include, but are not limited to, salts of edetate, (also known as salts of ethylenediaminetetraacetic acid, or EDTA, such a disodium EDTA) and natural antimicrobials.

In some embodiments, the food ingredient is formulated as a nutraceutical. In some embodiments, the food ingredient is formulated as a dietary supplement, food ingredient or additive, a medical food, nutraceutical or pharmaceutical composition. A food ingredient or additive is an edible substance intended to result, directly or indirectly, in its becoming a component or otherwise affecting the characteristic of any food (including any substance intended for use in producing, manufacturing, packing, processing, preparing, treating, packaging, transporting, or holding food). A food product, in particular a functional food, is a food fortified or enriched during processing to include additional complementary nutrients and/or beneficial ingredients. A food product according to this disclosure can, e.g., be in the form of butter, margarine, sweet or savory spreads, condiment, biscuits, health bar, bread, cake, cereal, candy, confectionery, soup, milk, yogurt or a fermented milk product, cheese, juice-based and vegetable-based beverages, fermented beverages, shakes, flavored waters, tea, oil, or any other suitable food. In some embodiments, the food product is a whole-food product in which the concentration of the compound has been enriched through particular post-harvest and food production processing methods to levels that provide an efficacious amount of the compound.

A dietary supplement is a product taken by mouth that contains a compound or extract of the disclosure and is intended to supplement the diet. A nutraceutical is a product derived from a food source that provides extra health benefits, in addition to the basic nutritional value found in the food. A pharmaceutical composition is defined as any component of a drug product intended to furnish pharmacological activity or other direct effect in the diagnosis, cure, mitigation, treatment, or prevention of disease or to affect the structure or any function of the body of humans or other animals. Dietary supplements, nutraceuticals and pharmaceutical compositions can be found in many capsules, forms such as tablets, coated tablets, pills, capsules, pellets, granules, softgels, gelcaps, liquids, powders, emulsions, suspensions, elixirs, syrup, and any other form suitable for use.

### Methods and Uses

An aspect of the disclosure includes methods for producing a food product. Other aspects of the disclosure include providing the food product to a subject.

One may also administer the food product or formulation described herein in a local rather than systemic manner. The food product or formulation may, if desired, be presented in a pack or dispenser device, which may contain one or more unit dosage forms containing the food product or formulation. The pack may, for example, comprise metal or plastic foil, such as a blister pack. Instructions for administration may accompany the pack or dispenser device. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs or the approved product insert. Compositions may include a compound and/or salt described herein formulated in a compatible pharmaceutical excipient may also be prepared, placed in an appropriate container, and labeled for treatment of an indicated condition.

The food product or formulation can be provided to an administering physician or other health care professional in the form of a kit. The kit is a package which houses a container which contains the compound(s) in a suitable pharmaceutical composition and instructions for administering the pharmaceutical composition to a subject. The kit can optionally also contain one or more additional therapeutic agents. The kit can also contain separate doses of a food product or formulation for serial or sequential administration. The kit can optionally contain one or more diagnostic tools and instructions for use.

The kit can contain suitable delivery devices, for example, syringes, and the like, along with instructions for administering the food product or formulation and any other therapeutic agent. The kit can optionally contain instructions for storage, reconstitution (if applicable), and administration of any or all food products or formulations included. The kits can include a plurality of containers reflecting the number of administrations to be given to a subject.

For preparing solid formulations such as tablets or capsules, the compound or extract is mixed with a carrier (e.g., conventional tableting ingredients such as corn starch, lactose, sucrose, sorbitol, talc, stearic acid, magnesium stearate, dicalcium phosphate or gums) and other diluents (e.g., water) to form a solid composition. This solid composition is then subdivided into unit dosage forms containing an effective amount of the compound of the present disclosure. The tablets or pills containing the compound or extract can be coated or otherwise compounded to provide a dosage form affording the advantage of prolonged action.

The liquid forms in which the compound or extract of the disclosure may be incorporated for oral or parenteral administration include an aqueous solution, suitably flavored syrups, aqueous or oil suspensions, and flavored emulsions with edible oils as well as elixirs and similar vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin. Liquid preparations for oral administration may take the form of, for example, solutions, syrups, or suspensions, or they may be presented as a dry product for reconstitution with water or other suitable vehicles before use. Such liquid preparations may be prepared by conventional means with acceptable additives such as suspending agents (e.g., sorbitol syrup, methyl cellulose, or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

Methods of preparing formulations or compositions of this disclosure may include a step of bringing into association a food product or formulation of the present disclosure with the carrier and, optionally, one or more accessory and/or active ingredients. In general, the food product or formulations are prepared by uniformly and intimately bringing into association a compound or extract of the present disclosure with liquid carriers, or finely divided solid carriers, or both, and then, if necessary, shaping the product. As such, the disclosed food product or formulation may consist of, or consist essentially of a compound or extract described herein in combination with a suitable carrier.

When a food product or formulation of the present disclosure is administered as pharmaceuticals, nutraceuticals, food additive, or dietary supplements to humans and animals, they can be given per se or as a food product, whole food ingredient, extract ingredient, pressed cake, liquid ingredient, or formulation containing one or more bioactive. A food product or formulation may be consumed by a subject to provide less than 100 mg of one or more bioactives disclosed herein per day. In certain embodiments, the food product or formulation provides between 10 and 60 mg/day of a tyramine containing hydroxycinnamic acid amide. The effective amount can be established by methods known in the art and be dependent upon bioavailability, toxicity, etc.

While it is contemplated that individual tyramine containing hydroxycinnamic acid amides may be used in the food product or formulation of this disclosure, it is further contemplated that two or more of the food products or formulations could be combined in any relative amounts to produce custom combinations of ingredients containing one or more bioactives in desired ratios to enhance product efficacy, improve organoleptic properties or some other measure of quality important to the ultimate use of the product.

This disclosure provides methods for improving, restoring, or maintaining digestive health. In accordance with such methods, an effective amount of a compound or extract of this disclosure is provided to a subject in need thereof so that the subject's digestive function is improved or maintained, thereby addressing the underlying pathogenesis of one or more chronic gastrointestinal disorders and promoting the health, well-being, and quality of life of the subject. In some embodiments, the subject is a veterinary, companion, farm, laboratory, or zoological animal. In other embodiments, the subject is a human.

In some embodiments, the food product or formulation improves the digestive health of a subject. In some embodiments, the food product or formulation ameliorates a disease or condition associated with digestive health in a subject. In some embodiments, treating or ameliorating digestive health in a subject is not a treatment or improving inflammation. In some embodiments, the food product or formulation ameliorates a disease or condition associated with digestive health in a subject by increasing HNF4α expression. In some embodiments, the food product or formulation treats or ameliorates a disease or condition associated with digestive health in a subject and reverses the loss of Paneth cells that occur from a high fat diet. In some embodiments, the food product or formulation treats or ameliorates a disease or condition associated with digestive health in a subject that increases intestinal villus. In some embodiments, the food product or formulation treats or ameliorates a disease or condition associated with digestive health in a subject by increasing Paneth cell formation. In some embodiments, the food product or formulation treats or ameliorates a disease or condition associated with digestive health in a subject by decreasing a condition associated with an allergic response.

In an embodiment, the food product or formulation treats or improves at least one factor associated with the digestive health of a subject. In other aspects, the food product or formulation improves the digestive health of a subject by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects, the food product or formulation improves digestive health by of reducing a disease or condition associated with digestive health from a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%. In some embodiments, disease or condition associated with the digestive health level of Paneth cells, HNF4α level, allergic response, or intestinal villus levels.

In an embodiment, the food product or formulation has an anti-inflammatory activity capable of reducing the levels of inflammation in the liver or intestine. In other aspects, the food product or formulation has an anti-inflammatory activity capable of reducing the levels of inflammation by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects, the food product or formulation has an anti-inflammatory activity capable of reducing the levels of inflammation in the liver or intestines from a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%. In some embodiments, the inflammation is chronic inflammation. In some embodiments, the composition reduces a symptom associated with inflammation. In some embodiments, the composition treats, reduces, or eliminates a symptom associated with inflammation in a subject. In some embodiments, the composition treats, reduces, or eliminates inflammation in a subject.

In some embodiments, inflammation symptoms may include, without limitation, edema, hyperemia, erythema, bruising, tenderness, stiffness, swollenness, fever, chills, stuffy nose, stuffy head, breathing problems, fluid retention, blood clots, loss of appetite, increased heart rate, formation of granulomas, fibrinous, pus, non-viscous serous fluid, or ulcer and pain.

In some embodiments, inflammation symptoms can be associated with a large, unrelated group of disorders that underlay a variety of diseases and disorders. In some embodiments, the immune system is often involved with inflammation disorders, demonstrated in both allergic reactions and some myopathies, with many immune system disorders resulting in abnormal inflammation.

A subject in need of a food product, whole food ingredient, or extruded ingredient of this disclosure includes a subject with observable symptoms of a chronic gastrointestinal disorder (e.g., a subject with abdominal pain, blood in stool, pus in the stool, fever, weight loss, frequent diarrhea, fatigue, reduced appetite, tenesmus, and rectal bleeding), as well as a subject who has no observable symptoms of a chronic gastrointestinal disorder but has been determined to be susceptible to developing the gastrointestinal disorder (i.e., a subject at risk of developing the gastrointestinal disorder).

The term “effective amount,” as used herein, means an amount of the compound, extract, or formulation containing the compound or extract which is sufficient to significantly improve a disorder. As used herein, the term “improve” or “improved” should be taken broadly to encompass improvement in an identified characteristic of a disease state, said characteristic being regarded by one of skill in the art to generally correlate, or be indicative of, the disease in question, as compared to a control, or as compared to a known average quantity associated with the characteristic in question. For example, “improved” digestive health associated with the application of a compound or extract of the disclosure can be demonstrated by comparing the digestive health (e.g., abdominal symptoms, stool, fever, weight, appetite, and/or epithelial barrier integrity) of a human treated with the compound or extract, as compared to the digestive health of a human not treated. Alternatively, one could compare the digestive health of a human treated with a compound or extract of the disclosure to the average digestive health of a human, as represented in scientific or medical publications known to those of skill in the art. In the present disclosure, “improved” does not necessarily demand that the data be statistically significant (i.e., p<0.05); rather, any quantifiable difference demonstrating that one value (e.g., the average treatment value) is different from another (e.g., the average control value) can rise to the level of “improved.”

Of concern when determining an effective amount to be used in humans is balancing the desired effects (benefits) against risks associated with the use of a compound. At issue for such risk/benefit assessments are the types of adverse effects observed and the likelihood that they will occur. Also considered is the fact that the effective amount may vary with the particular disorder being treated, e.g., IBD, IBS, UC, or CD, the age and physical condition of the end user, the severity of the condition, the duration of the treatment, the particular carrier utilized, and like factors.

In general, a suitable daily dose of a compound or extract of the disclosure will be that amount of a compound or extract, which is the lowest dose that is effective at producing a desired benefit, in this case, an effect that improves digestive health and consequently overall health and well-being. Such an effective dose will generally depend upon the factors described herein. For oral administration, the dose may range from about 0.0001 mg to about 10 grams per kilogram of body weight per day, about 5 mg to about 5 grams per kilogram of body weight per day, about 10 to about 2 grams per kilogram of body weight per day, or any other suitable dose. If desired, the effective daily dose of the compound or extract may be administered as two, three, four, five, six, or more sub-doses administered separately at appropriate intervals throughout the day, optionally, in unit dosage forms. In some embodiments, dosing is one administration per day.

The compound or extract of the disclosure can be used alone or in combination with a particular diet or standard of care. By way of illustration, a compound or extract of this disclosure may be combined with a gluten-free diet or used in combination with an aminosalicylate, a corticosteroid, a thiopurine, methotrexate, a JAK inhibitor, a sphingosine 1-phosphate (SIP) receptor inhibitor, an anti-integrin biologic, an anti-IL12/23R or anti-IL23 biologic, and/or an anti-tumor necrosis factor agent or biologic.

Administration of a compound or extract of the disclosure improves digestive function, thereby addressing the underlying pathogenesis of one or more gastrointestinal disorders and promoting the health, well-being, and quality of life of the subject. Ideally, an effective amount of a compound or extract provides a measurable improvement in the levels or activity of HNF4α activity and/or intestinal epithelial barrier and/or digestive function compared to a subject not receiving treatment. More particularly, the use of a compound or extract of the disclosure preferably prevents, slows the progression of, delays, or treats an intestinal disorder such as IBD, IBS, UC, and/or CD.

In some embodiments, the food product or formulation may be provided to treat a chronic gastrointestinal disorder. The terms “chronic gastrointestinal disorder,” “gastrointestinal epithelial cell barrier function disorder,” “chronic disease related to disruption of the intestinal epithelial barrier,” and the like refer to conditions in which individuals have a chronic or recurring immune response and inflammation of the gastrointestinal (GI) tract. The most common diseases or disorders are irritable bowel syndrome (IBS), inflammatory bowel disease (IBD), Crohn's disease (CD), ulcerative colitis (UC), and celiac disease. Other chronic gastrointestinal disorders include, but are not limited to, necrotizing enterocolitis, indeterminate colitis, chronic colitis, HIV enteropathy, Helicobacter gastritis, NSAID-enteropathy/enteritis, pouchitis, discontinuous or patchy disease, ileal inflammation, extracolonic inflammation, granulomatous inflammation in response to ruptured crypts, aphthous ulcers, transmural inflammation, microscopic colitis, diverticulitis, diversion colitis, short bowel syndrome, GI mucositis, chemotherapy-induced mucositis, radiation-induced mucositis, and interstitial cystitis.

In some embodiments, the food product or formulation may be provided to treat Ulcerative colitis. UC is a disease that causes inflammation and sores, called ulcers, in the lining of the large intestine. The inflammation usually occurs in the rectum and lower part of the colon, but it may affect the entire colon. UC may occur in people of any age and is believed to be the result of the body's immune system reacting to a virus or a bacterium by causing ongoing inflammation in the intestinal wall. People with ulcerative colitis have abnormalities of the immune system, but it has not been shown that these abnormalities are the cause or the result of the disease.

The most common symptoms of ulcerative colitis are abdominal pain and bloody diarrhea. Patients also may experience fatigue, weight loss, loss of appetite, rectal bleeding, and loss of body fluids and nutrients. About half of the patients have mild symptoms. Others suffer frequent fever, bloody diarrhea, nausea, and severe abdominal cramps.

In some embodiments, the food product or formulation may be provided to treat, prevent, or ameliorate Crohn's disease. Crohn's disease is characterized by intestinal inflammation and the development of intestinal stenosis and fistulas; neuropathy often accompanies these symptoms. In some instances, it is believed that Crohn's disease results from a failure of the intestinal mucosal barrier, possibly resulting from genetic susceptibilities and environmental factors (e.g., smoking), which exposes the immune system to antigens from the intestinal lumen, including bacterial and food antigens. Another hypothesis is that persistent intestinal infection by pathogens such as Mycobacterium paratuberculosis, Listeria monocytogenes, abnormal Escherichia coli, or paramyxovirus stimulates the immune response; alternatively, symptoms result from a dysregulated immune response to ubiquitous antigens, such as normal intestinal microflora and the metabolites and toxins they produce.

The presence of IgA and IgG anti-Saccharomyces cerevisiae antibodies (ASCA) in the serum has been found to be highly diagnostic of pediatric Crohn's disease. Further, in active cases of Crohn's disease, elevated concentrations of TNF-α and IL-6 are secreted into the blood circulation, and TNF-α, IL-I, IL-6, and IL-8 are produced in excess locally by mucosal cells. In this respect, it has been suggested that cytokine profiles in stool samples could be a useful diagnostic tool for Crohn's disease.

In some embodiments, the food product or formulation may be provided to treat, prevent, or ameliorate irritable bowel syndrome. IBS is a disorder that affects mainly the bowel or large intestine. IBS causes cramping, bloating, gas, diarrhea, and constipation. With IBS, the nerves and muscles in the bowel are extra sensitive and may be activated during or shortly after a meal or exercise, thereby causing cramping and diarrhea. Foods that cause symptoms include milk products, chocolate, alcohol, caffeine, carbonated drinks, and fatty foods. In some cases, simply eating a large meal will trigger symptoms.

In some embodiments, the food product or formulation may be provided to treat, prevent, or ameliorate necrotizing enterocolitis. Necrotizing enterocolitis is an acquired disease, primarily in premature infants or sick newborns, in which intestinal tissue dies. In necrotizing enterocolitis, the lining of the intestinal wall dies, and the tissue sloughs off. The cause for this disorder is unknown, but it is thought that a decreased blood flow to the bowel keeps the bowel from producing the normal protective mucus. Bacteria in the intestine may also be a cause. At risk are small, premature infants, infants who are fed concentrated formulas, infants in a nursery where an outbreak has occurred (suggesting an infectious cause), and infants who have received blood exchange transfusions.

Symptoms include abdominal distention, vomiting, lethargy, feeding intolerance, blood in the stool, temperature instability, and diarrhea. Diagnosis usually involves an abdominal x-ray, and examination for occult stool blood, elevated white count, thrombocytopenia, and lactic acidosis.

In some embodiments, the food product or formulation may be provided to treat, prevent, or ameliorate Celiac disease. Celiac disease is a digestive disease that damages the small intestine with absorption of nutrients from food. People who have digestive interference who have celiac disease cannot tolerate gluten, a protein found in wheat, rye, and barley. When people with celiac disease eat foods or use products containing gluten, their immune system responds by damaging the small intestine.

Celiac disease is a genetic disease that may be triggered or become active for the first time after surgery, pregnancy, childbirth, viral infection, or severe emotional stress. A subject with celiac disease may present with diarrhea and abdominal pain, irritability, depression, gas, recurring abdominal bloating, foul-smelling or fatty stool, weight loss/gain, fatigue, unexplained anemia, bone or joint pain, osteoporosis, osteopenia, behavioral changes, tingling numbness in the legs (from nerve damage), muscle cramps, seizures, missed menstrual periods (often because of excessive weight loss), infertility, recurrent miscarriage, delayed growth, failure to thrive in infants, pale sores inside the mouth (called aphthous ulcers), tooth discoloration or loss of enamel, and itchy skin rash (dermatitis herpetiformis). Celiac disease may be diagnosed using test blood for measuring levels of Immunoglobulin A (IgA), anti-tissue transglutaminase (tTGA), and IgA antiendomysium antibodies (AEA).

In some embodiments, the food product or formulation may be provided to treat, prevent, or ameliorate HIV enteropathy. HIV enteropathy is a syndrome characterized by chronic, well-established diarrhea (greater than one month in duration) without an identified infectious cause after a thorough evaluation in an HIV-positive individual. It is thought to be due to the direct or indirect effects of HIV on the enteric mucosa.

In some embodiments, the food product or formulation may be provided to treat, prevent, or ameliorate an infection. In some embodiments, the infection is Heliobacter pylori. Heliobacter pylori can cause infections of the stomach that may contribute to the development of dyspepsia (heartburn, bloating, and nausea), gastritis (inflammation of the stomach), and ulcers in the stomach and duodenum. H. pylori infection can be diagnosed by endoscopic biopsy followed by testing of the removed tissue for the bacteria, a breath test, or a blood test (measuring antibodies against these bacteria present in the blood). Symptoms include discomfort, bloating, nausea, and perhaps vomiting, as well as ulcers.

In some embodiments, the food product or formulation may be provided to treat, prevent, or ameliorate NSAID-Enterophay/Enteritis. The anti-inflammatory, analgesic, and antipyretic properties of NSAIDs are well-established and can be used in a wide range of disorders. A major limitation of NSAIDs' clinical utility is their gastroduodenal epithelial toxicity. NSAID toxicity is not site-specific to the gastroduodenum and can induce toxicity in the more distal intestine.

In some embodiments, a food product, whole food ingredient, or extruded ingredient of this disclosure may be provided to a subject to treat, prevent, improve, or maintain a subject's digestive health. In some embodiments, the subject is provided the food product, whole food ingredient, or extruded ingredient of this disclosure, thereby treating, improving, preventing, or maintaining the subject's digestive health. In some embodiments, a food product, whole food ingredient, or extruded ingredient of this disclosure may be provided to a subject to reverse hepatic steatosis in a subject. In some embodiments, the subject is provided the food product, whole food ingredient, or extruded ingredient of this disclosure, thereby reversing hepatic steatosis in the subject. In some embodiments, a food product, whole food ingredient, or extruded ingredient of this disclosure may be provided to a subject to treat obesity in a subject. In some embodiments, the subject is provided the food product, whole food ingredient, or extruded ingredient of this disclosure, thereby treating obesity in the subject. In some embodiments, a food product, whole food ingredient, or extruded ingredient of this disclosure may be provided to a subject to treat non-alcoholic fatty liver disease (NAFLD) in a subject. In some embodiments, the subject is provided the food product, whole food ingredient, or extruded ingredient of this disclosure, thereby treating NAFLD in the subject. In some embodiments, a food product, whole food ingredient, or extruded ingredient of this disclosure may be provided to a subject to treat non-alcoholic steatotic hepatitis (NASH) in a subject. In some embodiments, the subject is provided the food product, whole food ingredient, or extruded ingredient of this disclosure, thereby treating NASH in the subject. In some embodiments, a food product, whole food ingredient, or extruded ingredient of this disclosure may be provided to a subject to treat or maintain insulin levels in a subject. In some embodiments, the subject is provided the food product, whole food ingredient, or extruded ingredient of this disclosure, thereby treating or maintaining insulin levels in the subject. In some embodiments, a food product, whole food ingredient, or extruded ingredient of this disclosure may be provided to a subject to treat or maintain type-2 diabetes in a subject. In some embodiments, the subject is provided the food product, whole food ingredient, or extruded ingredient of this disclosure, thereby treating type-2 diabetes in the subject. In some embodiments, a food product, whole food ingredient, or extruded ingredient of this disclosure may be provided to a subject to reduce fat in a subject. In some embodiments, the subject is provided the food product, whole food ingredient, or extruded ingredient of this disclosure, thereby reducing fat in the subject. In some embodiments, reducing fat includes reducing belly fat. In some embodiments, providing the food product, whole food ingredient, or extruded ingredient promotes fat clearance from the liver.

### EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### Example 1

In this example, a new, continuous process for the extraction of bioactives from hemp hulls was performed. Specifically, this example aimed to obtain products with a higher percentage of bioactives than the unprocessed hemp hulls.

An extruder was constructed using a 13 barrel (LID=52.5) set-up, including an endplate. The main feed was positioned in barrel 1 via a feeder. A combi barrel was used for barrel 6 to side-feed the starch or flour. A combi barrel was used for barrel 11, along with a side feeder with an open barrel set upside down for the purpose of dewatering. Additional set-ups for dewatering will be discussed later. Water was injected mainly into barrel 7 for week #1 of the trial and into barrel 4 for week #2 of the trial. In week #1, a roasted and milled powdered product was obtained, as well as a product with a dough-like consistency and one with a puffed texture. In week #2, a press cake and a liquid extraction portion was obtained. The water was injected using a piston pump with a container on a scale.

At the very beginning of the trial, the focus was on first successfully roasting the whole hemp hulls, which was done using screw design as described herein. Data points for samples Roast 1-4 were obtained using a screw design described herein. The screw design was used to roast and also mill the whole hemp hulls to a desired particle size. The screw design was used to test different screw speeds and product formulations. In the screw design, an additional feed section was added for the addition of starch in the formulation. It was quickly realized that the product could handle more shear, so the screw design was designed to have more mixing/shearing sections. The last screw design of week #1 was designed to be more aggressive with stronger mixing sections and left-handed elements. The screw design was used during week #2 of the trial and was designed to attempt to separate the solid material from the liquid portion of the product. In all of these designs, a combi barrel was used in 11 with a dewatering set-up. The temperature profile of the barrels played an important role in whether some extraction was achieved in the open vacuum dome in barrel 11. At temperatures between 130° C.-150° C. some water separation could be seen in the open vacuum dome. At 100° C. a slurry of hemp hulls and water collected in the dome. Once the open vacuum dome filled up with this slurry material, the extruder would have to be shut down and cleaned out before another attempt at collection. This was because the slurry of hemp hulls and water would start to bubble in the vacuum dome and would inevitably explode after a few minutes. The successful temperature profiles included a temperature of 80° C. both near the water injection site and near the combi barrel dewatering set-up in barrel 11. Barrels 6 and 7 could be more elevated in temperature (130° C.-200° C.). High temperature, pressure, and shear are important components of this reaction. It was found that Barrels 6 and 7 needed to be heated above 130° C. to obtain the initial liquid separation in the dewatering barrel. The liquid was pipetted out of the vacuum dome, and samples were taken for further testing. The liquid separation was collected in the open vacuum dome. To prevent a slurry and hemp hulls from coming up into the open vacuum dome, a 40 mesh screen was placed between the open barrel and the vacuum dome as a temporary solution to assist in the collection of samples. The specific mechanical energy for the successful extraction portion of the trial fell between 0.028 and 0.140. These values will be used to scale up to a larger machine. Samples #35-36 (used whole hemp hulls) and #39-40 (used milled/roasted hulls) were the most successful in obtaining an extraction portion.

Formulation 1 represents only the roasted and milled material before the addition of water. The screw designs were used to test only the effectiveness of the roasting and milling unit operation. During this test, screw speed and throughputs were tested to understand the correlation between those two variables and how they affect the roasting characteristics and the final particle size. Slower screw speeds and higher throughputs created smaller particles in the milling process. The screw speeds of 50 RPM-100 RPM had 100% degree of fill, and the material was backing up in the vent. A satisfactory particle size was achieved by increasing throughput and screw speed (160 RPM). Formulations 2-10 consisted of either hemp hulls and water to form a paste-like product or hemp hulls, water, and starch to form a dough-like product. For this process, water was injected into barrel 7, and the unit operation from barrels 2 to 6 was roasting and milling. For this reason, lower screw speeds were favored to give the roasting and milling process more residence time. Additionally, lower screw speeds created a higher degree of fill in the extruder and, therefore, smaller particle sizes in the milling process. Samples of products with smaller, fine particulates and products with larger particles were collected for further testing. The screw design was used for this portion of the test. This design includes mixing in the roasting/milling section and mixing downstream to incorporate the addition of water and, in some formulations, corn starch. The addition of corn starch early on seemed to create slippage within the screws and prevented efficient milling of the hemp hulls. The particle size of the end-product was larger with the addition of corn starch in comparison to the formulation without the use of starch and with the same conditions. The open feed throat for the starch would experience backing up. Further, water was back flowing into the starch feeder with a screw design as described herein. The order in which the raw materials were fed was important to prevent the back flowing of the water. More specifically, hemp hulls should be fed first, then the starch, and finally, the water can be added slowly (1 pph per minute). An end product with a dough-like or moldable consistency was created using the described screw design. A formula of 23 lb/hr hemp hulls, 7 lb/hr starch, and 8 lb/hr water is the minimal formula needed to create a product with a moldable consistency.

Samples consisted of a formulation containing hemp hulls, water, and flour or soy protein. This formulation was used to create a puffed product. For this test, a die plate was used containing 8 mm×2 die inserts. The process was relatively stable and cohesive. A steady strand with some expansion was achieved, but the cell structure did not retain. A higher flour content creates good stranding (about 18%). Pea protein was also tested in this formulation. The protein did not bind as well to the material. The final product is drier than with the use of flour (also at 18%). But, even at 6% pea protein in the formulation, the final product is very dry and powdery. Pictured to the right is the puffed, stranded product using flour.

Samples focused on the extraction portion of the trial were also produced. For this test, barrel 11 was a combi barrel that was set up for dewatering. Three different set-ups were tested for dewatering. The first set-up that was tested consisted of a side feeder with an open barrel set upside down for dewatering. A slurry of hemp hulls and water collected under the upside-down barrel; meanwhile, a sludge came out through open discharge. The slurry produced was determined to be insufficient for customer needs. A similar set-up was tested, but instead of the upside-down open barrel being used for dewatering, an open vacuum dome attached to a valve was used. Another set-up was tested with an open vacuum dome attached directly to the open barrel 11. The design with the open vacuum dome attached directly to barrel 11 was used for the collection of all samples. A 40 mesh screen was inserted between the open barrel and the open vacuum dome to help keep the slurry of hemp hulls and water inside the extruder. A small amount of water separation containing bioactives collected in the vacuum dome opening, and the material was able to be pipetted into sample containers for further testing. The screw designs were used for the dewatering process, with the screw design was the most promising and the only design to achieve any noticeable liquid separation. A small amount of water extraction was possible using 5 lb/hr hemp hulls and 8 lb/hr water. The barrel temperatures of 6 and 7 were important to this test. Barrels 6 and 7 needed to be between 130° C.-150° C. for water to collect in the vacuum dome. Alternatively, the previously roasted and milled hemp hulls were tested in this process. When using the milled hemp hulls, extraction was seen at 17 lb/hr hulls and 16 lb/hr water. Decreasing the screw speed to 400 RPM from 600 RPM appeared to increase the rate at which the extracted material collects in barrel 11. The liquid that separated out had a pleasant, sweet aroma.

The extruder successfully created product(s) with a higher percentage of bioactives than the unprocessed hemp hulls using the screw design at a throughput rate between 15 lb/h-46 lb/h and an extruder screw speed of 600 RPM. The roasted and milled hemp hulls were used during the most successful liquid extraction trial runs (two-pass system). The screw design was used to roast and mill the whole hemp hulls, and the screw design was used for the extraction portion of the trial.

The liquid extraction was achieved by utilizing an open barrel with an open vacuum dome and a bucket for collecting material. A mesh screen was used in between barrel 11 and the open vacuum dome as a temporary solution to help the customer more easily collect samples of the liquid extraction portion of the extrudate. Otherwise, a slurry of material would pool into the opening, along with some occasional separation of the liquid portion. A small amount of extraction collects in the open vacuum dome when the following conditions are met; barrels 6 and 7 are heated between 130° C.-150° C., water is injected into barrel 4, screw speed is about 600 RPM, and water is used at about a 2 to 1 ratio to the whole hemp hulls or about a 1 to 1 ratio to the roasted/milled hemp hulls.

A finished product containing a dough-like or puffed texture is attainable with the addition of starch and or flour to the formulation.

### Example 2

Experiments were performed to roast hemp hulls to improve sensory attributes, milling feasibility, and bioactive bioaccessibility.

For this example, the extruder roasting temperature for barrels #1-5 were set at 250° C., barrel #6 at 100° C., and barrels #7-13 were set at ambient temperature. The screw speed was set to 50-100 rpm. The feed rate was 10 lb/hr of hemp hulls. The product provides 100% degree of fill in the screws with no water.

The final product from this example produced a nutty/toasty/sesame aroma. The color was light brown, and the texture was crispy.

### Example 3

Experiments were performed to determine an efficient milling process for a whole food ingredient powder.

For this example, an aggressive screw design was tested with three sets of 2 kneading blocks to provide high shearing/intense milling unit operations. The adjustments to screw speed and hemp hull feed rate produced a range of whole food ingredient particle sizes at 200° C. at 70 rpm, and 10 lb/hr hemp hull feed rate produced very fine powder (similar to a spray dried powder). An increase to 160 rpm reduced screw fill and produced a larger particle size. Addition of water at 9 lb/h at 250° C., 160 rpm, and 23 lb/h hemp hull to fine particle size with approximately 23% water.

### Example 4

Experiments were performed to add excipients as structure-function enablers for hemp hull whole food ingredient products.

For this example, the use of structure-function enablers improved the functionality of bioactives. The steam explosion (puffing) and excipients increased bioaccessibility. The melt flow increased dissolution properties in the presence of excipients. Steam-enabled direct expansion with excipients: water only produced a fine particle size at 450 rpm, 250° C., 23 lb/h hemp hull, and 9 lb/h water, cornstarch at 1.5 lb/h (3% formulation) produced a gelatinous hemp hull whole food ingredient strand, but the steam explosion did not occur; flour at 7 lb/h, water at 26 lb/h, hemp hull at 23 lb/h, and 7 mm die produced continuous, direct-expanded hemp hull whole food ingredients strands; and soy protein concentrate at 23 lb/h produced no puffing and less hemp hump with more soy protein concentrate produced a crisp.

### Example 5

Experiments were performed to achieve phase separation in an extruder to guide the extraction process.

For this example, side feeders were added to convey liquid from an extruder. It was observed that this hindered pressure of the extruder. A hopper was added to barrel #8 to collect fractions. The side feeders were removed, and a vacuum dome/vent pipe was added to collect a liquid fraction. A 40-mesh screen was added to filter the liquid. The extruder was run at 600 rpm, 5 lb/h hemp hulls, 8 lb/h water, and the barrels were at 130° C. to 150° C. The product produced was a turbid green extract from whole hemp hulls. The product produced was a brown, translucent extract using milled hemp hulls.

### Example 6

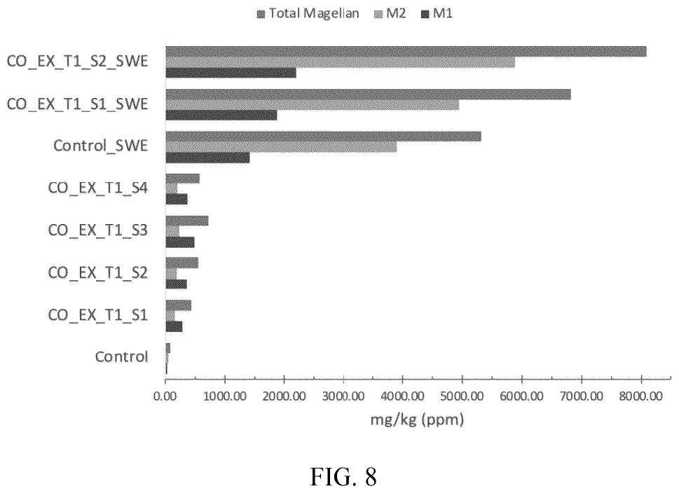

Experiments were performed to determine the yields of bioactives from whole food ingredient powders. Using an extruder described herein utilizing a roasting/milling of hemp hulls at 250° C. and 200 rpm. ASE SWE yielded >8000 ppm total n-trans-caffeoyltyramine/n-trans-feroyltyramine, a 52% increase relative to SWE control. This result was compared to an ASE ethanol extraction. The ASE ethanol extraction yielded >8000 ppm total bioactive, a 52% increase relative to SWE control. It was determined that extrusion improved the release of bioactive from the hemp full matrix, and SWE maximized yields. The ethanol extraction of hemp hull whole food ingredients was not a sufficient workstream compared to current extrusion conditions. The results of this trial are illustrated in FIG. 8.

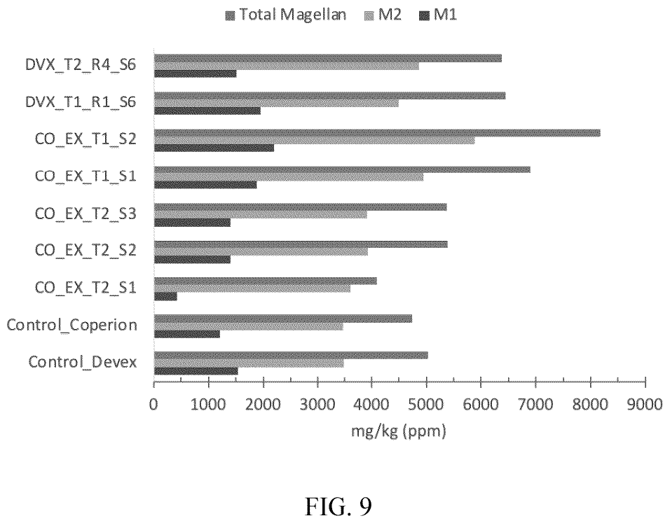

Experiments were also performed to compare the extrusion of hemp hulls paired with SWE improved bioactive yields. Using an extruder as described herein as compared to a first trial at 190° C., 15 barg (218 psi), 1:10 solvent:feed ratio. It was determined that the extruded material contains higher bioactive content than unprocessed feed after SWE. The extruder, as described herein, produced a yield of 54% increase compared to the control, whereas the first trial produced a 25% increase compared to the control. The results of this trial are illustrated in FIG. 9.

Experiments were also performed to compare phase separation in cold extrusion and an extruder, as described herein. For this experiment, samples were collected and quantified on a dry basis using the assumption of 1 hour run time. On average, 66% of bioactive was extracted from the press cake. Most bioactive was left behind in the press cake rather than the extract. The loss of the bioactive in uncollected liquid phase was determined. The results of this trial are illustrated in Table 1.

TABLE 1

| | n-trans- | n-trans- | Total |
|---|---|---|---|
| | caffeoyltyramine | feruloyltyramine | Bioactive |
| Sample ID | (mg/kg) | (mg/kg) | (mg/kg) |
| CONTROL | 1179 | 3768 | 4947 |
| CO_EX_T1_S2 | 2201 | 5889 | 8090 |
| CO_EX_T1_S3 | 1363 | 3888 | 5251 |
| CO_EX_T2_S5_PC | 775 | 2266 | 3041 |
| CO_EX_T2_S6_PC | 942 | 2929 | 3871 |
| CO_EX_T2_S7_PC | 787 | 2373 | 3160 |
| CO_EX_T2_S8_PC | 736 | 2347 | 3083 |
| CO_EX_T2_S5_EX | 182 | 156 | 338 |
| CO_EX_T2_S6_EX | 17 | 16 | 33 |
| CO_EX_T2_S7_EX | 406 | 171 | 577 |
| CO_EX_T2_S8_EX | 839 | 280 | 1119 |

### Example 7

In this example, extrusion sample parameters are provided describing the extrusion extracts, the extrusion press cake, and the extruded powders.

TABLE 2

| | | | Barrel | | Screw | Additional |
|---|---|---|---|---|---|---|
| | Details | Feed Rates | Temp | | Speed | Parameters |
| Extrusion extracts | Twice-extruded | 17 lb/hr HH, | 80-150-70° | C. | 600 | 15% torque, |
| | hemp hulls (HH) | 16 lb/hr water | | | rpm | 2.0 kW |
| | From Whole HH | 5 lb/hr HH, | 80-100-70° | C. | 600 | 25% torque, |
| | | 8 lb/hr water | | | rpm | 3.0 kW |
| | From Whole HH | 23 lb/hr HH, | 80-200-85° | C. | 600 | 16% torque |
| | | 23 lb/hr water | | | rpm | |
| | From Whole HH | 17 lb/hr HH, | 80-150-80° | C. | 600 | 14% torque |
| | | 16 lb/hr water | | | rpm | with 40- |
| | | | | | | mesh filter |
| | | | | | | screen |
| Extrusion press cake | Twice-extruded | 17 lb/hr HH, | 80-150-70° | C. | 600 | 15% torque, |
| (spent solids of | hemp hulls | 16 lb/hr water | | | rpm | 2.0 kW |
| matching extract | From Whole HH | 5 lb/hr HH, | 80-100-70° | C. | 600 | 25% torque, |
| above) | | 8 lb/hr water | | | rpm | 3.0 kW |
| | From Whole HH | 23 lb/hr HH, | 80-200-85° | C. | 600 | 16% torque |
| | | 23 lb/hr water | | | rpm | |
| | From Whole HH | 17 lb/hr HH, | 80-150-80° | C. | 600 | 14% torque |
| | | 16 lb/hr water | | | rpm | with 40- |
| | | | | | | mesh filter |
| | | | | | | screen |
| Extruded powders | Roasting/extruding | 23 lb/h HH | 250° | C. | 220 | |
| (milling unit | whole HH | | | | rpm | |
| operation)extracted | Roasting/extruding | | 250° | C. | 200 | |
| with ETHANOL | whole HH | | | | rpm | |
| | Roasting/extruding | 23 lb/h HH | 250-90° | C. | 325 | |
| | whole HH with | | | | rpm | |
| | more shear | | | | | |
| | Roasting/extruding | | 250° | C. | 200 | |
| | whole HH, 50% | | | | rpm | |
| | water with no | | | | | |
| | starch | | | | | |
| Extruded powders | Roasting/extruding | 23 lb/h HH | 250° | C. | 220 | |
| (milling unit | whole HH | | | | rpm | |
| operation) extracted | Roasting/extruding | | 250° | C. | 200 | |
| with subcritical water | whole HH | | | | rpm | |
| (SWE) | | | | | | |

### Example 8

In this experiment, a proximate analysis of hemp hulls was performed. It was determined that the extrusion process minimally impacts most macromolecular components of extruded materials. Even under shear and heat conditions, extrusion protects hemp hull components and increases the accessibility of components for analysis. The results of this experiment are described in Table 3.

TABLE 3

| | | | | | | Total |
|---|---|---|---|---|---|---|
| | Moisture | Fat | Protein | Ash | Total | dietary |
| Sample | (% w/w) | (% w/w) | (% w/w) | (% w/w) | carbs | fiber |
| Control, untreated | 6.91 | 7.90 | 11.60 | 2.46 | 71.13 | 78.1 |
| whole HH for | | | | | | |
| extrusion trials | | | | | | |
| Extruded HH - small | 4.83 | 7.06 | 11.35 | 2.54 | 74.22 | 73.9 |
| particle size | | | | | | |
| Extruded HH - large | 6.53 | 6.86 | 11.23 | 2.53 | 72.85 | 75.9 |
| particle size | | | | | | |
| Whole HH - Lot | 7.43 | 10.79 | 12.12 | 2.58 | 67.08 | 72.2 |
| Grower A | | | | | | |
| Whole HH - Lot | 6.51 | 16.82 | 15.43 | 3.79 | 57.45 | 62.9 |
| Blended | | | | | | |
| Milled HH | 7.36 | 2.56 | 9.75 | 2.66 | 77.67 | 78.7 |
| Whole HH | 6.55 | 15.09 | 17.6 | 5.7 | 55.06 | 65.1 |

### Example 9

In this experiment, a fiber analysis of hemp hulls was performed. The extrusion process was determined to increase hemicellulose content in hemp hulls, making it more available for analysis. Cellulose depolymerizes at 160-180° C., but extrusion at higher temperatures (>180° C.) shows no depolymerization/degradation of cellulose. Low molecular weight dietary fiber content is not increasing, suggesting that polysaccharide fiber components are not degrading into oligosaccharides. The results of this experiment are described in Tables 4 and 5.

TABLE 4

| | Dietary fiber (g/100 g) | | | | |
|---|---|---|---|---|---|
| | High | High | Integrated | | Acid Detergent Fiber |
| | molecular | molecular | TOTAL DF | Low | (% w/w) (cellulose, |
| | weight | weight | (insoluble + | molecular | lignin, insoluble |
| Sample | insoluble DF | soluble DF | soluble) | weight DF | minerals) |
| Control | 77.4 | 0.7 | 78.1 | <0.6 | 65.9 |
| (untreated | | | | | |
| whole HH) | | | | | |
| Extruded | 75.4 | 0.5 | 75.9 | <0.6 | 58.0 |
| HH: Small | | | | | |
| particle size | | | | | |
| Extruded | 75.4 | 0.5 | 75.9 | <0.6 | 61.7 |
| HH: Large | | | | | |
| particle size | | | | | |

TABLE 5

| | Fiber Components | | | | |
|---|---|---|---|---|---|
| Sample | Pectin | Hemicellulose | Cellulose | Beta-glucan | Lignin |
| Control | 0.4 | 10 | 43.0 | <0.1 | 22.9 |
| (untreated | | | | | |
| whole | | | | | |
| HH) | | | | | |
| Extruded | 0.26 | 18.1 | 40.4 | <0.1 | 17.6 |
| HH: Small | | | | | |
| particle | | | | | |
| size | | | | | |
| Extruded | 0.22 | 15.2 | 39.5 | <0.1 | 22.2 |
| HH: Large | | | | | |
| particle | | | | | |
| size | | | | | |

### Example 10

In this experiment, an analysis of the material properties of hemp hulls was performed. It was determined that the bulk density of hemp hulls depends on particle size, which can be controlled within the extruder. The results of this experiment are described in Table 6.

TABLE 6

| | Material Properties | | | |
|---|---|---|---|---|
| | Bulk | Water Activity | Peroxide | Particle |
| Sample | Density | at 25° C. | Value | Size |
| Control | 0.25 g/ml | 0.355 | 0.29 mEq/kg | — |
| (untreated | TAPPED | | | |
| whole HH) | | | | |
| Extruded | 0.59 g/ml | 0.289 | 3.68 mEq/kg | Median size |
| HH: Small | TAPPED | | | (d50): 445 μm |
| particle | | | | |
| size | | | | |
| Extruded | 0.56 g/ml | 0.429 | 8.71 mEq/kg | Median size |
| HH: Large | TAPPED | | | (d50): 581 μm |
| particle | | | | |
| size | | | | |
| Whole HH: | 0.29 g/ml | 0.392 | 3.47 mEq/kg | — |
| Lot | TAPPED | | | |
| Grower A | | | | |
| Whole HH: | 0.27 g/ml | 0.362 | 0.40 mEq/kg | — |
| Lot | TAPPED | | | |
| Blended | | | | |
| Milled HH | 0.68 g/ml | 0.447 | 1.06 mEq/kg | — |
| | TAPPED | | | |
| Whole HH | 0.23 g/ml | 0.333 | 2.58 mEq/kg | — |
| | TAPPED | | | |

### Example 11

In this experiment, an analysis of the material properties of hemp hulls was performed. It was determined that the extrusion process increases the mineral contents of hemp hulls compared to the untreated material due to improved accessibility. The results of this experiment are described in Table 6.

TABLE 6

| | Minerals (ppm, w/w) | | | | | | | |
|---|---|---|---|---|---|---|---|---|
| Sample | Ca | Cr | Cu | Fe | Mn | Mg | Mo | K |
| Control | 1530 | 0.34 | 15.5 | 75.8 | 63.9 | 1570 | 0.26 | 3850 |
| (untreated | | | | | | | | |
| whole | | | | | | | | |
| HH) | | | | | | | | |
| Extruded | 1710 | 1.43 | 16.1 | 106 | 71.1 | 1690 | 0.40 | 4200 |
| HH: | | | | | | | | |
| Small | | | | | | | | |
| particle | | | | | | | | |
| size | | | | | | | | |
| Extruded | 1730 | 1.31 | 16.6 | 117 | 69.7 | 1670 | 0.43 | 4410 |
| HH: | | | | | | | | |
| Large | | | | | | | | |
| particle | | | | | | | | |
| size | | | | | | | | |

### Example 12

In this experiment, the trace mineral contents of hemp hulls were performed. The extrusion process was determined to increase trace mineral contents hemp hulls compared to the untreated material due to improved accessibility for analysis. The results of this experiment are described in Table 7.

TABLE 7

Trace minerals (ppm, w/w

| Sample | Al | Ba | Ni | Sr |
|---|---|---|---|---|
| Control | 5.0 | 7.60 | 0.48 | 17.5 |
| (untreated | | | | |
| whole HH) | | | | |
| Extruded | 5.7 | 7.96 | 0.54 | 18.5 |
| HH: Small | | | | |
| particle size | | | | |
| Extruded | 5.4 | 8.22 | 0.58 | 19.3 |
| HH: Large | | | | |
| particle size | | | | |

### Example 13

In this experiment, phenolic contents of hemp hulls were performed. The extrusion process was determined to increase the phenolic contents of hemp hulls compared to the untreated materials due to improved accessibility for analysis and availability of compounds from the hemp hull matrix. The results of this experiment are described in Table 8.

TABLE 8

| | Total phenolic | |
|---|---|---|
| Sample | content | |
| Control (untreated whole HH) | 8,140 | μg GAE/g |
| Extruded HH: Small particle size | 10,800 | μg GAE/g |
| Extruded HH: Large particle size | 8,160 | μg GAE/g |

### Example 14

In this experiment, THC, CBD, and cannabinoid content were analyzed. It was determined that the extrusion process increases the availability of THC, CBD, and cannabinoid compounds for analysis, but overall contents remain low. Milled material does not show high contents of these compounds, suggesting that the extrusion process provides more shear to release the compounds from the hemp hull matrix and make them more available for extraction and/or accessibility. It is believed that the increase in the THC/CBD/cannabinoids in hemp hulls provides a “fingerprint” for the extrusion process. The results of the experiment are described in Tables 9, 10, and 11.

TABLE 9

| Sample | THCV | THCVA | THCA | Δ8-THC | Δ9-THC |
|---|---|---|---|---|---|
| Control | <1.00 | <1.00 | <1.00 | <1.00 | <1.00 |
| (untreated | | | | | |
| whole HH) | | | | | |
| Extruded | <1.00 | <1.00 | 1.15 | <1.00 | 1.72 |
| HH: Small | | | | | |
| particle size | | | | | |
| Extruded | <1.00 | <1.00 | 1.17 | <1.00 | 1.59 |
| HH: Large | | | | | |
| particle size | | | | | |
| Whole HH: | <1.00 | <1.00 | <1.00 | <1.00 | <1.00 |
| Lot Grower | | | | | |
| A | | | | | |
| Whole HH: | <1.00 | <1.00 | <1.00 | <1.00 | <1.00 |
| Lot Blended | | | | | |
| Milled HH | <1.00 | <1.00 | <1.00 | <1.00 | <1.00 |
| Whole HH | <1.00 | <1.00 | <1.00 | <1.00 | <1.00 |

THCV-tetrahydrocannabivarin;
THCVA-tetrahydrocannbinolic acid;
THCA-tetrahydrocannabinolic acid

TABLE 10

| Sample | CBDV | CBNA | CBCA | CBD | CBL |
|---|---|---|---|---|---|
| Control | 1.07 | <1.00 | 1.03 | 14.9 | <1.00 |
| (untreated | | | | | |
| whole HH) | | | | | |
| Extruded | 2.65 | <1.00 | 2.16 | 38.2 | 1.72 |
| HH: Small | | | | | |
| particle size | | | | | |
| Extruded | 2.50 | <1.00 | 2.14 | 37.2 | 1.59 |
| HH: Large | | | | | |
| particle size | | | | | |
| Whole HH: | <1.00 | <1.00 | <1.00 | 10.9 | <1.00 |
| Lot Grower | | | | | |
| A | | | | | |
| Whole HH: | <1.00 | <1.00 | <1.00 | 13.8 | <1.00 |
| Lot Blended | | | | | |
| Milled HH | <1.00 | <1.00 | <1.00 | <1.00 | <1.00 |
| Whole HH | <1.00 | <1.00 | <1.00 | 1.24 | <1.00 |

CBDV-Cannabidivarin;
CBNA-Cannabinolic Acid;
CNBA-Cannabinolic Acid;
CBC-Cannabichromene;
CBD-Cannabidiol

TABLE 11

| Sample | CBDA | CBDVA | CBG | CBGA | CBN |
|---|---|---|---|---|---|
| Control | 44.9 | 3.44 | <1.00 | 3.44 | <1.00 |
| (untreated | | | | | |
| whole HH) | | | | | |
| Extruded | 51.2 | 3.09 | <1.00 | 3.09 | <1.00 |
| HH: Small | | | | | |
| particle size | | | | | |
| Extruded | 49.3 | 3.15 | <1.00 | 3.15 | <1.00 |
| HH: Large | | | | | |
| particle size | | | | | |
| Whole HH: | 35.5 | 2.24 | <1.00 | 2.24 | <1.00 |
| Lot Grower | | | | | |
| A | | | | | |
| Whole HH: | 34.7 | 2.20 | <1.00 | 2.20 | <1.00 |
| Lot Blended | | | | | |
| Milled HH | 2.58 | <1.00 | <1.00 | <1.00 | <1.00 |
| Whole HH | 8.76 | <1.00 | <1.00 | <1.00 | <1.00 |

CBDA-Cannabidiolic Acid;
CBDVA-Cannabidivarinic Acid;
CBG Cannabigerol;
CGBA-Cannabigerolic Acid;
CBN Cannabinol

### Example 15

In this experiment, the amino acid profile of hemp hulls was analyzed. It was determined that regardless of the high processing temperatures applied in the extruder (250° C.), the hemp hull amino acid profiles are not impacted. The stability of the amino acid profile in extruded hemp hull powders offers a “fingerprint” of the product related to the use of the extrusion process. The results of the experiment are described in Tables 12 and 13.

TABLE 12

| | Amino Acids (% w/w) | | | | | | | | | |
|---|---|---|---|---|---|---|---|---|---|---|
| Sample | A | V | M | L | D | Y | W | I | P | HP |
| Control | 0.41 | 0.42 | 0.1 | 0.61 | 0.97 | 0.23 | 0.04 | 0.34 | 0.51 | 0.09 |
| (untreated | | | | | | | | | | |
| whole | | | | | | | | | | |
| HH) | | | | | | | | | | |
| Extruded | 0.40 | 0.48 | 0.09 | 0.67 | 1.11 | 0.20 | 0.04 | 0.34 | 0.51 | 0.12 |
| HH: | | | | | | | | | | |
| Small | | | | | | | | | | |
| particle | | | | | | | | | | |
| size | | | | | | | | | | |
| Extruded | 0.41 | 0.49 | 0.1 | 0.69 | 1.14 | 0.24 | 0.04 | 0.36 | 0.54 | 0.19 |
| HH: | | | | | | | | | | |
| Large | | | | | | | | | | |
| particle | | | | | | | | | | |
| size | | | | | | | | | | |

HP—hydroxy proline

TABLE 13

| | Amino Acids (% w/w) | | | | | | | | |
|---|---|---|---|---|---|---|---|---|---|
| Sample | R | H | F | K | G | S | T | E | C |
| Control | 0.77 | 0.21 | 0.46 | 0.36 | 0.43 | 0.44 | 0.34 | 0.04 | 0.14 |
| (untreated | | | | | | | | | |
| whole | | | | | | | | | |
| HH) | | | | | | | | | |
| Extruded | 0.64 | 0.19 | 0.46 | 0.32 | 0.41 | 0.42 | 0.35 | 1.70 | 0.24 |
| HH: | | | | | | | | | |
| Small | | | | | | | | | |
| particle | | | | | | | | | |
| size | | | | | | | | | |
| Extruded | 0.71 | 0.20 | 0.47 | 0.34 | 0.42 | 0.43 | 0.37 | 1.93 | 0.23 |
| HH: | | | | | | | | | |
| Large | | | | | | | | | |
| particle | | | | | | | | | |
| size | | | | | | | | | |

### Example 16

In this experiment, the effects of particle size on hemp hulls were analyzed. The extruded hemp hulls were added to a sieve stack and shaken until particles were no longer falling through the mesh screens. It was determined that the highest bioactive content lies within 250-2000 μm particle sizes. The results of the experiment are described in Table 14.

TABLE 14

| | | | | | | Total | |
|---|---|---|---|---|---|---|---|
| | | | | | Total | Bioactive | |
| | Bioactive (ppm) in the sieve particle size | | | | Bioactive | of non- | |
| | ranges | | | | Mass | sieved | |
| | 500-2000 | 250-500 | 125-250 | 63-125 | Fractions | material | Process |
| Sample | μm | μm | μm | μm | (ppm) | (ppm) | Parameters |
| HH WFI | 7339 | 8130 | 5624 | 1856 | 6788 | 5338 | 400 rpm |
| small | | | | | | | 25 1b/hr |
| particle | | | | | | | S.D #7 |
| size | | | | | | | |
| Twice- | 0 | 8507 | 5661 | 3358 | 6300 | 4040 | 600 rpm |
| extruded | | | | | | | 27 lb/hr |
| powder | | | | | | | S.D #3/11 |
| CO- | 8932 | 7952 | 4277 | 1823 | 6809 | 8090 | 200 rpm |
| EX_T1_S2 | | | | | | | 25 1b/hr |
| | | | | | | | S.D #6 |

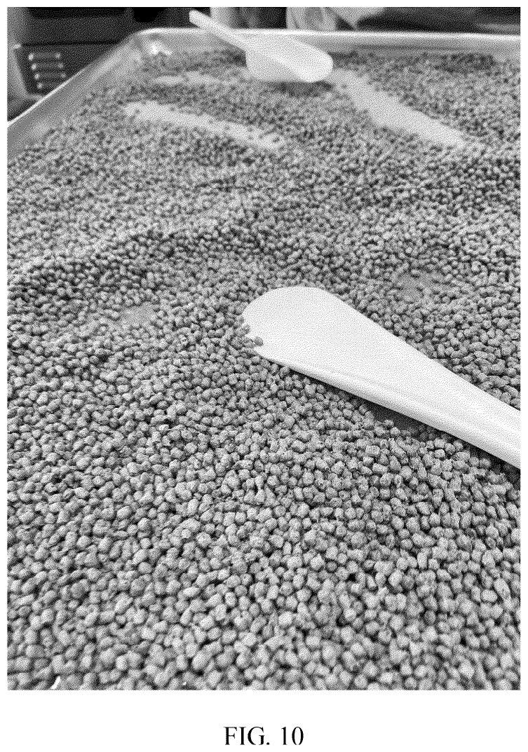

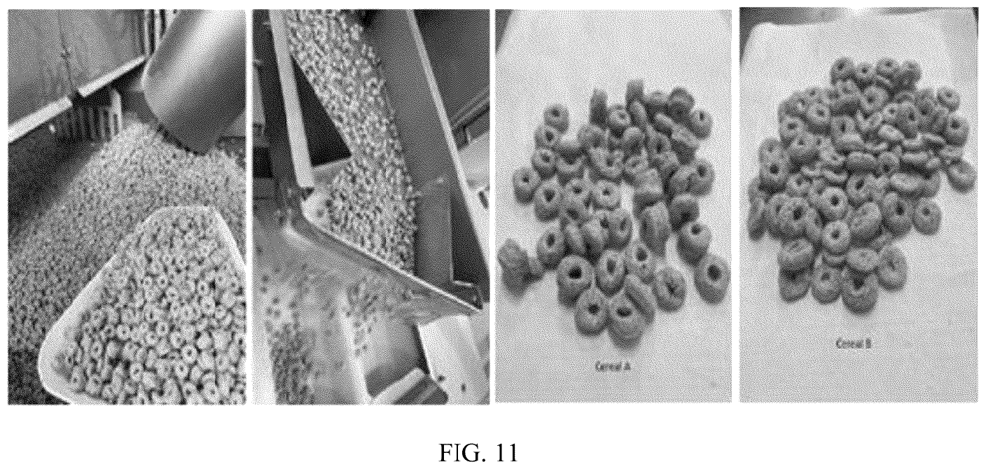

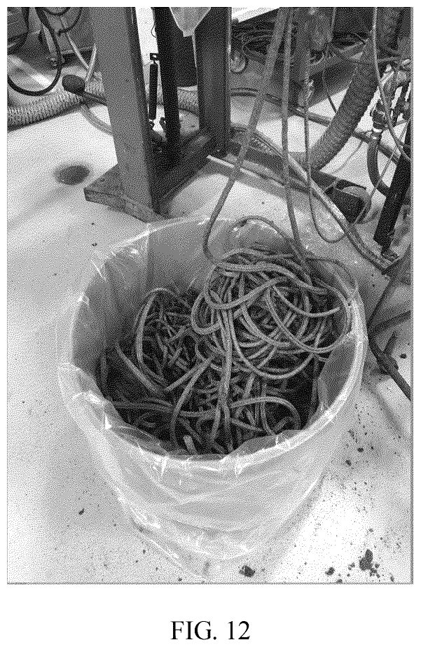

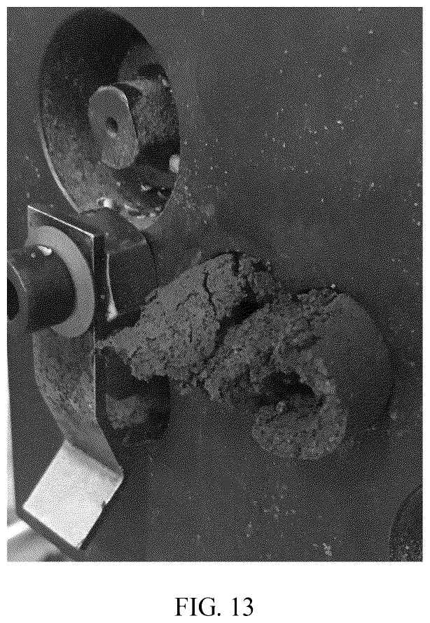

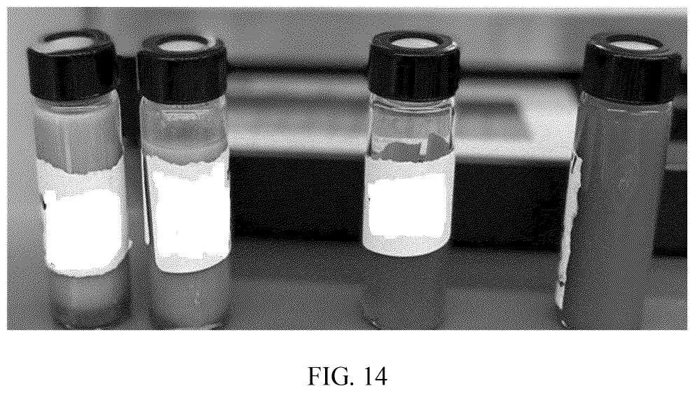

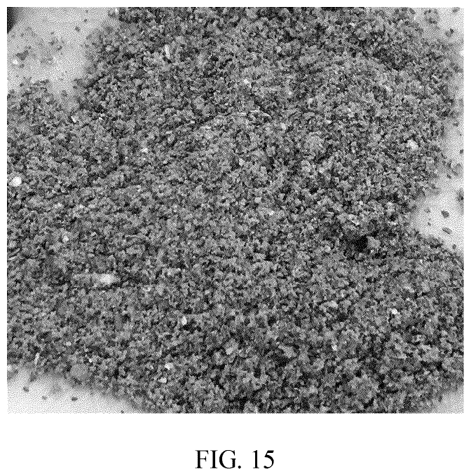

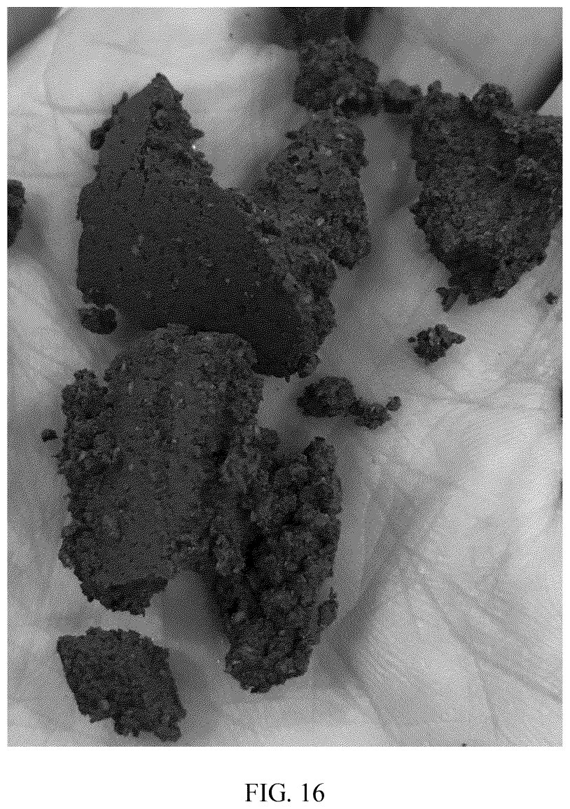

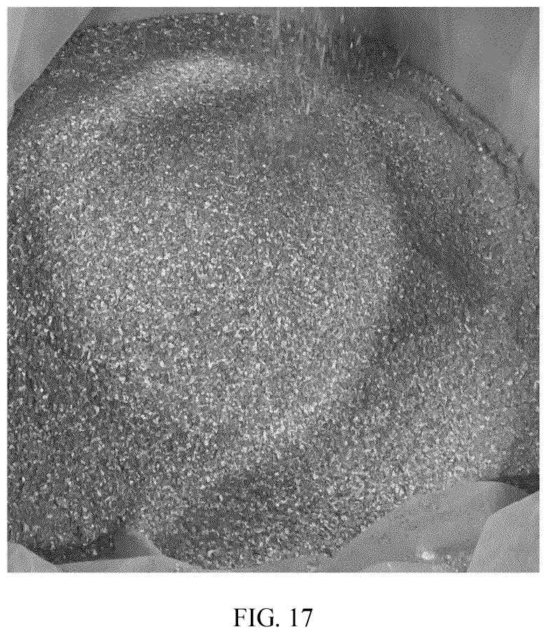

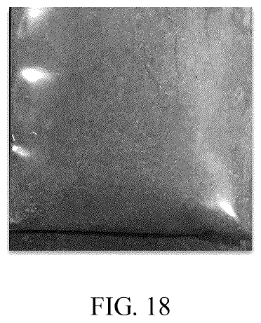

In the following examples, new cereal, crisp, and crostini products were produced. Specifically, this example aimed to validate the successful continuous production of Hemp Food Ingredients incorporated into Cereals, Crisps, & Crostinis. Optimizations were performed on the screws, flow rates, feed rates, and other parameters in order to vary the properties of the feed material and the efficiency of the extruder. The following examples were performed using the following equipment: a MVIRMNA “SaniMix” mixer, a K-Tron T32 gravimetric feeder (Main Feeder) Stuffer screw to force powder into an extruder, a BCTG Polytwin™ twin-screw extruder, 62 mm diameter, 20:1 L/d, w/rotary cutter, a 224 kW (300 hp) drive at 1000 RpM maximum screw speed High Sanitary Single-pass Dryer, 2 heating zones and 1 cooling zone SCBU “UniMill”mill (for destruction of dried waste product), and a bulk bag packaging system with inline metal detection, modified for bulk box filling. Table 15 describes example recipes used in Examples 17 through 20, to produce cereal, crisp, and crostini products incorporating hemp hull fiber. FIGS. 10 to 18 illustrate photos of products in accordance with the disclosure.

TABLE 15

| | Cereal A | Cereal B | Crisp A | Crisp B | Crostini |
|---|---|---|---|---|---|
| Ingredients | (%) | (%) | (%) | (%) | (%) |
| Hemp Hulls | 12 | 12 | 12 | 12 | 12 |
| Powder | | | | | |
| (~50% fiber) | | | | | |
| Soy Protein | 28 | 0 | 59 | 59 | 0 |
| Concentrate | | | | | |
| Yellow Corn | 57.4 | 85.4 | 27.5 | 0 | 0 |
| Flour | | | | | |
| All Purpose | 0 | 0 | 0 | 27.5 | 86.5 |
| Wheat Flour | | | | | |
| Coloring | 0.1 | 0.1 | 0 | 0 | 0 |
| Seasoning | 0 | 0 | 0 | 0 | 1 |
| Sugar | 2 | 2 | 1 | 1 | 0 |
| CaCO₃ | 0.5 | 0.5 | 0.5 | 0.5 | 0.5 |

### Example 17

In this example, the processability of the hemp hulls was verified. The extruder was started up using 50 kg/h hemp hulls, 0% water, and 150 rpm extruder speed. Barrel temperatures should be set as follows: 2-3, 4-5, 6-7, and 8-9 set for 200° C., 200° C., 180° C., and 120° C. The feed rate was slowly increased to 25 kg/h per time until 100 kg/h was achieved while also increasing the extruder screw speed up to 300 rpm. The hemp hull powder was observed exiting from the extruder and was considered acceptable. The powder was observed to have a green color, which in some embodiments is less desired. The material was collected for about 3 hours onto metal trays to allow brief cooling before being put into lined fiber drums.

A particle size distribution of the product was collected using Rotap, and the results are displayed in Table 16. The Moisture content was observed to be 2.32%.

TABLE 16

| Mesh (μm) | 18 (1000) | 30 (595) | 45 (354) | 70 (210) | 100 (149) | 140 (105) | pan |
|---|---|---|---|---|---|---|---|
| % Retained | 12.3% | 63.3% | 82.2% | 98.8% | 99.0% | 99.0% | 100% |
| (Cumulative) | | | | | | | |

This example included the possibility for adjustments to the screw profile by adding additional polygon blocks at the end for more grinding/crushing work into the material; however, adjustments were not made during this example.

### Example 18

In this example, the processability of the hemp hulls to create adequate cereals was verified. The extruder was started using Cereal A dry blend, as shown in Table 16. The dry blend contained 9.61% moisture. The extruder was started up with 150 kg/h, 250 rpm extruder speed, and 40% water. Production mode was 200 kg/h, 350 rpm extruder speed, and 25% water. Continue decreasing water until 8% water is achieved. The cutter speed was 2500 rpm. A target of 180° C. end plate temperature would show good expansion out of the die. Changed to Cereal B on the fly. Cereal products were observed to have satisfactory characteristics. Results from both samples can be seen in Table 17.

TABLE 17

| Sample | | |
|---|---|---|
| Blend | Cereal A | Cereal B |
| Bulk Density (grams/2 Liters) | 132.4 | 116.9 |
| Moisture out of extruder (%) | 8.62 | 9.59 |
| Moisture out of dryer (%) | 2.44 | 2.74 |

### Example 19

In this example, the processability of the hemp hulls to create adequate crisps was verified. The extruder was started after adding a stuffer screw into the feed throat and relocating a water injection port. The process efficiency was improved after these changes. Blend for Crisp A was made first, then switched to Crips B blend on the fly. Results from both samples can be seen in Table 18.

TABLE 18

| Sample | | |
|---|---|---|
| Blend | Crisp A | Crisp B |
| Bulk Density (grams/2 Liters) | 289.1 | 296.8 |
| Moisture out of extruder (%) | 14.55 | 14.32 |
| Moisture out of dryer (%) | 3.04 | 3.01 |

### Example 20

In this example, the processability of the hemp hulls to create adequate crostinis was verified. The extruder was operated at 200 kg/h and 300 rpm extruder speed. Water addition was 16%, and the cutter speed was set to 700 rpm to make a good product but thin enough to dry through the dryer. The process needed to be heated slowly for the ropes to expand well. All products were observed to have satisfactory characteristics.

### Example 21

In this example, suitable roasting of raw whole food ingredients was verified. This example aimed to determine extrusion parameters that may achieve adequate roasting of the raw hemp hulls to facilitate milling. Successful roasting was observed to enable fine particle sizes. Roasting also improved sensory attributes and bioaccessibility of bioactives. Surprisingly, approximately double the bioactive concentration of bioactives was observed in roasted HEMP HULL compared to the control.

Example parameters to provide sufficient roasting include a roasting temperature of 250° C. (barrels #1-5 at 250° C. with barrel 6 at 100° C. and barrels 7-13 at ambient temperature), while Barrel 4 had an open vent. A successful roasting was characterized by a change in aroma from green/grassy to nutty/toasty/sesame, a slight change in color from green/white to brown, and a crisper texture. High roasting and milling efficiency were achieved by modifying the extruder's fill rate, which is anticipated to increase the shear forces on the whole food ingredient. One example of a desired fill rate was tested with a 50 rpm screw speed and 10 lb/hr feed rate (hemp hull throughput). These parameters were observed to provide 100% degree of fill to the process. This example also verified a screw design consisting of conveying elements with a small-pitch section that increased fill.

### Example 22

In this example, the suitable particle size of roasted hemp hulls without prior milling was verified. This experiment aimed to determine extrusion parameters that may reduce the particle size of roasted hemp hulls, thus avoiding the need for external milling unit operation upstream of extrusion. It was observed that extrusion conditions enabled desired fine particle size (and various sizes) using roasting and shear elements. The screw design conveyed elements with three sets of 2 kneading blocks (3-lobed) to provide shearing/milling operations. A temperature of 220° C. in the first few barrels with a screw speed of 70 rpm at 10 lb/hr produced a fine powder. The extruder had vents that could be closed to build more pressure in the extruder and change the stress conditions. The experiment progressed by increasing the roasting barrel temperature to 250° C. and increasing the screw speed to 160 rpm at 23 lb/hr to achieve fine particle size. Here, the suitable particle size of roasted hemp hulls without prior milling was verified. This example also successfully explored the use of a higher throughput water pump to increase the water flow rate to create a slurry/dough. In another example, water was injected at ˜23% formulation at 9 lb/hr.

### Example 23

In this example, puffing for a HEMP HULL whole food ingredient product was verified. One aim of this experiment was to make a slurry/dough using milled HEMP HULL (within the extruder) to enable puffing for a HEMP HULL whole food ingredient product. Whole, untreated HEMP HULL was used to develop a HEMP HULL whole food ingredient puff with the addition of water and/or excipients. At least two different water pumps were tested, which allowed ˜30 lb/hr, as compared to the previous pump (8-9 lb/hr). Additionally, during this example, the screw design was updated to add more kneading elements downstream to further increase shearing and reduce particle size. Operating the extruder at 180 rpm without water at 250° C. produced a fine particle size. Once the water was added, fine particle size was produced at 450 rpm, 250° C., 46.5% water, 23 lb/h HEMP HULL, and 9 lb/h H2O. While in continuous operation, cornstarch was optionally added as a binding agent. While in continuous operation, all-purpose flour was optionally added as a binding agent, and better-puffing action than starch was observed. Continuous puffed strands (that did not break) shows the stability of HEMP HULL WFI matrix during the puffing action.

Puffed strands were generated using a 7 mm die (containing two openings) with 23 lb/h HEMP HULL, 7 lb/h flour, and 26 lb/h water. While in continuous operation, optional changes to different dies, die sizes, and process conditions provided an opportunity to improve whole food ingredient puffs.

While in continuous operation, soy protein concentrate was optionally added as a binding agent. However, during this run at a 23 lb/h soy feed rate, the HEMP HULL whole food ingredient was not initially wet enough to compress or puff. Accordingly, changes to the ratios of water and feedstocks (HEMP HULL and soy protein concentrate) were made to affect the puffing ability. Specifically, while in continuous operation, the inclusion of less HEMP HULL and more soy protein concentrate was indicated for successful puffs.

### Example 24

In this example, two feedstocks were tested. First, extrusion-treated hemp hulls (roasted and milled), and second whole hemp hulls (untreated). This experiment aimed to mill the whole HEMP HULL to gather enough extrusion-treated material to be used as a feedstock for further EI development. An intense shear screw design was used to successfully create a fine particle size (a powder with satisfactory particle size was observed). Some successful extrusion conditions used in this example to produce extrusion-treated (milled) HEMP HULL were 400 rpm, 25 lb/h HEMP HULL feed, and 250-90-85° C. barrel temperatures (85° C. at the end of the extruder).

### Example 25

In this example, the liquid fraction was separated from the solid press cake/spent feed. One aim of this experiment was to begin an EI process. Example setpoints that were observed to produce a good paste with a particle size similar to spray dried powder particle sizes were: Barrels 2-5 (120° C.), Barrels 7-13 (85° C.), side feeder at barrel 8 at 362 rpm, extruder at 600 rpm, Torque 15%, and a HEMP HULL feed at 7 lb/hr, with a water feed rate of 23 lbs/hr. During this experiment, another screw design with aggressive kneading blocks and left-handed elements was tested, and successfully built pressure while maintaining high shear. In another configuration, the extruder successfully used two side feeders (one as an outlet for liquid and one as an additional hopper). In particular, one screw design was tested that produced satisfactory without needing starch, flour, or soy protein. This design output a nice press cake with suitable shaping characteristics and good compressibility. With the new screw design, the extruder produced very fine milling of the hemp hulls, even with the addition of water at the beginning of the extruder. Press-cake (fiber-rich) was also produced simultaneously at the end of the extruder. During the operation of this example, the general conditions were: 100-150° C. upstream barrels, 80° C. downstream barrels, 400 rpm screws, 17 lb/h HEMP HULL, and water feeds. In another configuration, the conditions were: 600 rpm, 5 lb/h HEMP HULL, 8 lb/h water, 3.0 kW, and 25% torque.

In some trial runs of this example, liquid undesirably builds up in the tube and may be vented out of the extruder as a green/gray slurry. Additionally, some “burping” through the hopper was observed where the liquid fraction was pooling up, likely due to pressure and steam within the extruder. These issues were successfully overcome, and satisfactory fine powders and satisfactory liquid fractions were produced.

### Example 26

In this example, a study was undertaken to evaluate an extrusion process of an embodiment of the disclosure. For this study, hemp hull fiber as a CCP in the destruction of Salmonella spp was analyzed to determine if a 5-log reduction would result from the process.

Three separate batches of raw hemp hulls were provided. Product arrived at the lab in excellent condition and initial product moisture and water activity (Aw) values of each batch were obtained. For the thermal treatment validation, Enterococcus faecium NRRL B-2354 also designated as E. faecium ATCC 8459 was employed as a surrogate organism with equal to or greater heat resistance than Salmonella. This surrogate organism has also been used in thermal processing validations for a variety of nut, seed, flour, and other low moisture matrices.

Each product batch was inoculated with a separate culture batch. All culture batches were grown and harvested according to the Almond Board of California protocol to maximize thermal resistance as follows: a. Streak culture (active or frozen) onto Tryptic Soy Agar (TSA) plates i. Incubate at 35° C. for 24±2 hours; b. Transfer cells from isolated typical colonies into Tryptic Soy Broth (TSB) (10 mL), i. Incubate at 35° C. for 24±2 hours; c. Transfer loop of broth culture into TSB (10 mL),i. Incubate at 35° C. overnight (18±2 hours); d. Spread overnight culture (1 mL/plate) over large TSA plates (150×15 mm), i. Incubate at 35° C. for 24±2 hours; and e. Add 6 mL of 0.1% peptone to each plate, loosen bacterial lawn with a sterile spreader, and use a sterile pipette to collect cells into a sterile container.

The raw hemp hull batches were then inoculated and stabilized. Stabilization lasted until each product was no greater than 0.05 water activity (Aw) or 1% moisture above its original, uninoculated state. A lower stabilized moisture and water activity provides the inoculated bacteria with greater resistant to thermal lethality and provides a “worst case” for the study. Pre- and postinoculation moistures and water activities for the Hemp Hull batches were as described in Table 19.

TABLE 19

| Raw Hemp | Pre-inoculation | Post-inoculation | Pre-Inoculation | Post-Inoculation |
|---|---|---|---|---|
| Hull Batch | Moisture % | Moisture % | Water Activity | Water Activity |
| Batch 1 | 8.56 | 6.45 | 0.44 | 0.27 |
| Batch 2 | 7.79 | 5.37 | 0.40 | 0.21 |
| Batch 3 | 10.45 | 5.55 | 0.55 | 0.20 |

After stabilization, the lab tested two separate 25 g sample of each product batch. Samples were plated onto Tryptic Soy Agar, incubated for 2 hours and overlaid with KF Streptococcus agar, incubated at 35° C. for 48 hours prior to enumeration. The level of each inoculated sample was at least 107 per gram. The heat resistance of the inoculum on the product was also checked prior to the on-site validation study. A reduction of less than 2.5 logs after the prescribed heating trial (280° F. for 15 minutes) indicated the appropriate heat resistance of the surrogate culture. Pre- and post-heating inoculum levels were as described in Table 20.

TABLE 20

| Raw Hemp | Max. Initial | Min. Post-Heating | |
|---|---|---|---|
| Hull Batch | Inoculum Level | Inoculum Level | Difference |
| Batch 1 | 8.20 logs | 7.50 logs | 0.70 logs |
| Batch 2 | 8.12 logs | 7.69 logs | 0.43 logs |
| Batch 3 | 7.17 logs | 7.77 logs | 0.40 logs |

On the day of the validation trial, a Deibel Labs representative was onsite to conduct the study. For the trials, 45 lbs. of inoculated raw hemp hulls (combined from all three inoculated batches) were blended into a 529 lb. test batch for 30 minutes. Three adg blended control samples were collected from the batch to determine the initial surrogate concentration. Processing parameters for each trial are described in Table 21.

TABLE 21

| | Trial 1 Parameters | | Trial 2 Parameters | |
|---|---|---|---|---|
| | (Steady State) | | (Steady State) | |
| Extruder | 22 | Hz | 22 | Hz |
| Screw Speed | | | | |
| Product | 13 | Hz | 13 | Hz |
| Feed Rate | | | | |
| Water Added | 0.4 | lbs/hr | 0.4 | lbs/hr |

| | | |
|---|---|---|
| Zone 1 | Off/51.4-58.3° C. | Off/59.2-60.2° C. |
| Temperature | | |
| (Set/Actual) | | |
| Zone 2 | 160° C./155.5-171.9° C. | 160° C./159.2-160.7° C. |
| Temperature | | |
| (Set/Actual) | | |
| Zone 3 | 210° C./202.3-220.7° C. | 210° C./207.9-211.8° C. |
| Temperature | | |
| (Set/Actual) | | |
| Zone 4 | 210° C./204.1-215.8° C. | 210° C./206.8-212.2° C. |
| Temperature | | |
| (Set/Actual) | | |
| Zone 5 | 180° C./178.6-182.2° C. | 180° C./178.5-180.9° C. |
| Temperature | | |
| (Set/Actual) | | |
| Zone 6 | 160° C./159.1-160.9° C. | 140° C./130.1-147.2° C. |
| Temperature | | |
| (Set/Actual) | | |
| Zone 7 | 130° C./121.4-136.1° C. | 100° C./99.3-101.1° C. |
| Temperature | | |
| (Set/Actual) | | |

The extruder was evaluated for this validation. It is a twin screw designed for low moisture extruded products. The extruder has 7 zones. Six of the 7 zones are electrically heated. All heated zones are electrically heated, and water cooled. The modular barrel segments are jacketed for cooling. City water is used to cool the barrels. Electric solenoid valves control the amount of cooling to each barrel. All cooling water goes down the drain. Dry hemp hulls are delivered to the feed section of the extruder via volumetric twin screw feeder. Water is added during the startup procedure briefly. Water is added during the first 11 minutes of startup. Stable product is reached around the 40 minute mark. Material from the feeder is conveyed and cooked as it passes through the extruder. There is no die for this process. As product exits the extruder barrel it is collected in tubs. Tubs are passed through the metal detector and transferred to baking sheets for cooling. Trays are placed on a racking system for cooling before final bagging of the product.

During the validation, the extrusion room was divided into a raw side and finished product side. Operators on the raw side, loaded and operated the extruder. Personnel on the finished product side, collected the processed product as it exited the extruder and recorded validation parameters.

Sample collection pans were sanitized and labeled before samples were collected as they exited the extruder. Sterile scoops were used to aseptically transfer samples to sterile Whirl-Pak® bags and immediately chilled by placing the samples into a cooler with icepacks. A total of eighteen ˜50 g samples were collected under the first parameter setting: six early extrusion samples and twelve steady state samples. A total of fifteen ˜50 g samples were collected under the second parameter setting: three early extrusion samples and twelve steady state samples.

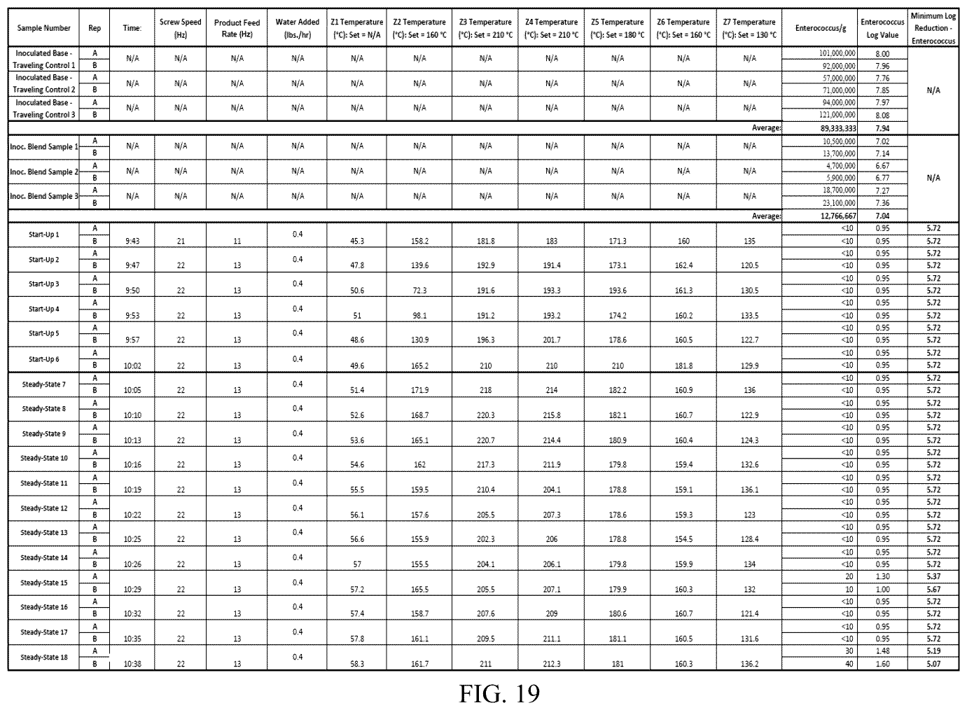

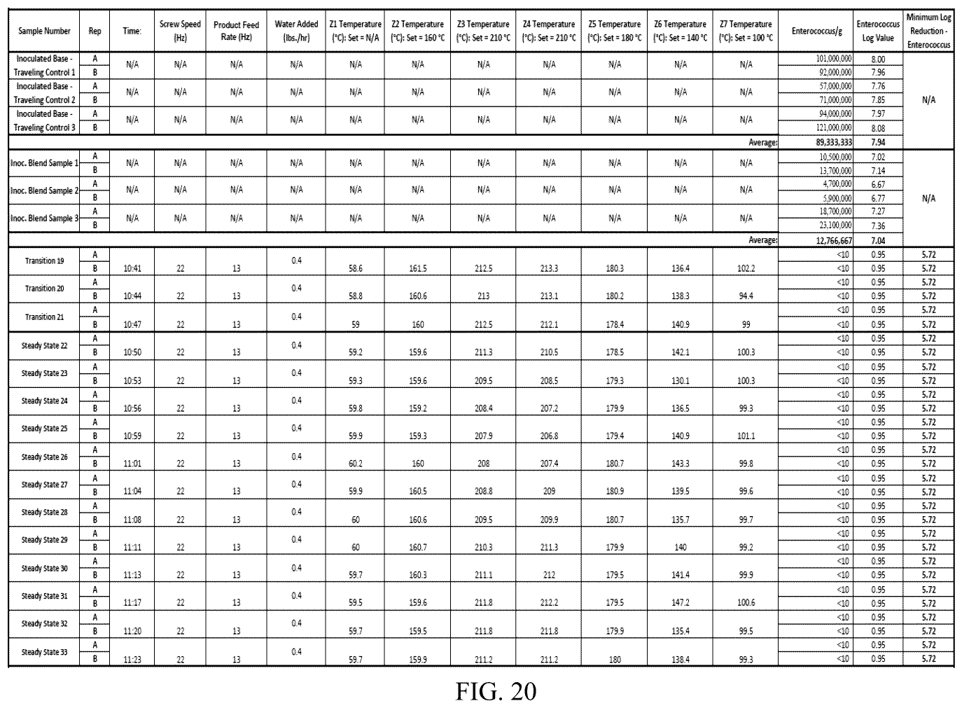

All trial samples, three blended traveling controls and three traveling controls were shipped, under refrigeration. Each lab sample was diluted with sterile phosphate buffer, stomached thoroughly, and plated, in duplicate, for direct counts of surviving cells on Tryptic Soy Agar, incubated for 2 hours and overlaid with KF Streptococcus agar. Agar plates were incubated at 35° C. for 48 hours prior to enumeration. Plates were counted and minimum log reduction was calculated for each test series. Results are presented in FIG. 19 and FIG. 20.

For this microbial challenge study, raw Hemp Hulls were inoculated with E. faecium. This organism has been found to be an acceptable surrogate for Salmonella spp. when used for processing validations in a variety of low moisture foods. E. faecium has typically demonstrated equal or higher heat resistance than Salmonella. Therefore, a reduction of 5 log E. faecium would be equivalent to a 5+ log reduction of Salmonella spp. Log reduction for the trials were as follows: Trial 1=5.69 logs and Trial 2=5.72 logs. The Hemp Hull Fiber extrusion process performed validated the 5-log minimum reduction in potential Salmonella under the parameters employed by this study. The data overwhelmingly supported this conclusion.

### Example 27

In this example, an extruder and extrusion process includes the following parameters:

Water Added=0.4 lbs./hr Minimum

Product Feed Rate: 13 Hz Minimum

Screw Speed: 22 Hz Minimum

Heated Zone Temperatures—(May meet or exceed the settings from Example 26): Zone 2=160° C., Zone 3=210° C., Zone 4=210° C., Zone 5=180° C., Zone 6=140° C., and Zone 7=100° C.

The extruder must be properly maintained and calibrated so zone temperature variability does not exceed what was observed for the validation trials

### Example 28

In this example, a yogurt, according to the disclosure, includes 68.7% by weight almond milk, 21.9% of cashew milk, 3.35% of coconut cream, 4.75% of a whole food ingredient, 1.18% of dextrose, 0.05% of locust bean gum, 0.05% of pectin, and 0.02% of live bacterial cultures customary for yogurt preparations, such as mixtures of lactic acid producing bacteria Lactobacillus bulgaricus and Streptococcus thermophilus. For a frozen dessert, example of amounts of a whole food ingredient may include about 4 g whole food ingredient per 79 g serving or 6.67 g whole food ingredient per 85 g serving.

### Example 29

In this example, an ice cream includes 45.5% by weight of water, 32% of coconut cream (34.7% fat), 4.5% of a whole food ingredient, 17% of sugar, 0.6% of gum, 0.2% of lecithin, 0.2% of sea salt.

### Example 30

In this example, a milk-based beverage includes 2.7 g whole food ingredients per 240 mL serv, 4 g carbohydrates, which may include 1 g of sugar, and 4 g of fat or oil from any source.

Accordingly, some aspects described herein relate to the following numbered alternatives:

1. An integrated extruder, comprising, a twin screw barrel assembly, and two co-rotating screws housed within the twin screw barrel assembly.

2. The integrated extruder of alternative 1, wherein the twin screw barrel assembly further comprises one or more feeder, compounder, mixer, reactor, or a combination thereof.

3. The integrated extruder of alternative 1 or 2, wherein the twin screw barrel assembly further comprises a heater or cooling system.

4. The integrated extruder of any one of alternatives 1 to 3, wherein the twin screw barrel assembly further comprises a mesh filter.

5. The integrated extruder of any one of alternatives 1 to 4, wherein the twin crew barrel assembly comprises a plurality of barrels which are connected to each other.

6. The integrated extruder of alternative 5, wherein the plurality of barrels may comprise a combi barrel with a hole in the side allowing for an additional input or an additional output.

7. The integrated extruder of any one of alternatives 1 to 3, wherein the twin screw barrel assembly comprises a first feeder positioned adjacent to a barrel configured to allow material to be input into the twin barrel assembly.

8. The integrated extruder of alternative 7, wherein the two screw barrel assembly comprises a second feeder positioned adjacent to a combi barrel configured to allow for the input of feed material into the twin barrel assembly.

9. The integrated extruder of any one of alternatives 1 to 8, further comprising a die plate positioned at the end of the barrel assembly.

10. The integrated extruder of any one of alternatives 1 to 9, further comprising a pressure release opening valve attached to the twin screw barrel assembly.

11. The integrated extruder of any one of alternatives 1 to 10, further comprising a water injection element attached to the twin screw barrel assembly.

12. A process for preparing a food composition, comprising: roasting hemp hulls to form roasted hemp hulls, milling roasted hemp hulls to form milled roasted hemp hulls, and feeding milled roasted hemp hulls into an integrated extruder as described herein or any one of alternatives 1 to 11 to form a hemp hull extract ingredient.

13. The process of alternative 12, further comprising pre-treating the milled roasted hemp hulls with water, enzymes, acid, base, or a combination there.

14. The process of alternative 12 or 13, further comprising high shear mixing the milled roasted hemp hulls.

15. The process of any one of alternatives 12 to 14, further comprising applying a high pressure extraction to the milled roasted hemp hulls.

16. The process of any one of alternatives 12 to 15, further comprising separating one or more solids and hemp hull extracts from the milled roasted hemp hulls.

17. The process of any one of alternatives 12 to 16, wherein roasting hemp hulls is at a temperature range from about 225° C. to about 275° C.

18. The process of any one of alternatives 12 to 18, wherein the milling roast hemp hulls is at speed from about 150 rpm to about 250 rpm.

19. A process for preparing a food composition, comprising roasting hemp hulls to form roasted hemp hulls, milling roasted hemp hulls to form milled roasted hemp hulls, and feeding milled roasted hemp hull into an integrated extruder as described herein or of any one of alternatives 1 to 11 to form a hemp hull whole food ingredient or hemp hull puff or crisp.

20. The process of alternative 19, further comprising pre-treating the milled roasted hemp hulls with water, enzymes, acid, base, or a combination there.

21. The process of alternative 19 or 20, further comprising high shear mixing the milled roasted hemp hulls.

22. The process of any one of alternatives 19 to 21, further comprising puffing, pelletizing or shaping the milled roasted hemp hulls.

23. The process of any one of alternatives 19 to 22, further comprising vacuum drying the milled roasted hemp hulls.

24. The process of any one of alternatives 19 to 23, further comprising granulating the milled roasted hemp hulls.

25. The process of any one of alternatives 19 to 24, wherein roasting hemp hulls is at a temperature range from about 225° C. to about 275° C.

26. The process of any one of alternatives 19 to 25, wherein the milling roast hemp hulls is at speed from about 150 rpm to about 250 rpm.

27. A food composition, the composition comprising extruded hemp hulls, wherein the extruded hemp hulls retain one or more bioactives from an extrusion process as described in alternatives 12 to 18 or in alternatives 19 to 26.

28. The food composition of alternative 27, wherein the food composition is a liquid product.

29. The food composition of alternative 27, wherein the food composition is an extract ingredient.

30. The food composition of alternative 27, wherein the food composition is a whole food ingredient.

31. The food composition of alternative 27, wherein the food composition is a pressed cake.

32. The food composition of alternative 27, wherein the food composition is a cracker.

33. The food composition of alternative 27, wherein the food composition is a cereal.

34. The food composition of alternative 27, wherein the food composition is an extruded puff.

35. The food composition of any one of alternatives 27 to 32, wherein the one or more bioactives is a tyramine containing hydroxycinnamic acid amide.

36. The food composition of alternative 33, wherein the tyramine containing hydroxycinnamic acid amide is selected from the group consisting of N-trans-caffeoyltyramine, N-cis-caffeoyltyramine, N-trans-feruloyltyramine, N-cis-feruloyltyramine, p-coumaroyltyramine.

37. The food composition of any one of alternatives 27 to 34, further comprising one or more micronutrients.

37. The food composition of any one of alternatives 27 to 35, further comprising one or more acceptable excipient or carrier for oral consumption.

38. A method for making a liquid product, the method comprising adding hemp hulls to an extruder as described in any one of alternatives 1 to 11 at a speed from about 500 rpm to about 700 rpm, wherein the hemp hulls are added at a rate from about 3 lb/h to about 8 lb/h, wherein water is added from about 5 lb/h to about 10 lb/h, wherein the temperature is from about 130° C. to about 150° C.; and screening the hemp hulls with a mesh screen, thereby collecting the liquid product.

39. A method for making an expanded food product, the method comprising forming a dough by admixing water and hemp hulls, extrusion cooking the dough to form a cooked dough, extruding the cooked dough through an extruder as described herein or any one of alternatives 1 to 11 to form cooked dough pieces, and cutting the cooked dough pieces, wherein the cooked dough pieces retain one or more bioactives.

40. A method of producing a hemp hull containing composite food product, the method comprising extruding hemp hulls with an extruder as described herein or any one of alternatives 1 to 11, thereby forming a milled hemp hull, and adding the milled hemp hull to addition food ingredients, thereby producing a hemp hull containing composite food product.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more’ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed:

1. A food product, comprising:
a hemp fiber;
a protein source;
a sugar;
a flour;
N-trans-caffeoyltyramine; and
N-trans-feruloyltyramine.

2. The food product of claim 1, further comprising a fat source.

3. The food product of claim 1 or 2, further comprising a vitamin or mineral.

4. The food product of claim 1 or 2, wherein the hemp fiber is a hemp hull fiber.

5. The food product of claim 1 or 2, wherein the hemp fiber is between about 10% and about 20% of the food product.

6. The food product of claim 1 or 2, wherein the protein source is soy protein.

7. The food product of claim 6, wherein the protein source is between about 25% and about 60% (w/w) of the food product.

8. The food product of claim 1 or 2, wherein the flour is between about 25% and about 90% (w/w) of the food product.

9. The food product of claim 1 or 2, wherein the sugar is between about 1% and about 5% (w/w) of the food product.

10. The food product of claim 1 or 2, further comprising calcium carbonate.

11. The food product of claim 9, wherein the calcium carbonate comprises between about 0.1% and about 1% (w/w) of the food product.

12. The food product of claim 1 or 2, wherein the N-trans-caffeoyltyramine comprises about 0.01% to about 20% (w/w) of the food product.

13. The food product of claim 1 or 2, wherein the N-trans-feruloyltyramine comprises about 0.01% to about 20% (w/w) of the food product.

14. The food product of claim 1 or 2, wherein the food product contains less than about 1% THC (w/w) of the food product.

15. The food product of claim 1 or 2, wherein the food product comprises about 2% to about 10% (w/w) moisture of the food product.

16. The food product of claim 2, wherein the fat source comprises about 5% to about 20% (w/w) of the food product.

17. The food product of claim 1 or 2, wherein the food product comprises about 1% to about 10% (w/w) ash of the food product.

18. The food product of claim 1 or 2, wherein the food product has a carbohydrate to dietary fiber ratio from about 2:1 to about 1:2.

19. The food product of claim 18, wherein the food product has a carbohydrate to dietary fiber ratio from about 1:1.

20. The food product of claim 1 to 19, wherein the food product is formulated as a bar, cracker, baked good, cereal, granola, functional beverage, or powdered beverage mix.

21. A plant-based crisp food product, comprising:
a pea protein;
a pea starch;
a hemp fiber;
N-trans-caffeoyltyramine; and
N-trans-feruloyltyramine.

22. The plant-based crisp food product of claim 21, further comprising a vitamin or mineral.

23. The plant-based crisp food product of claim 21 or 22, wherein the hemp fiber is a hemp hull fiber.

24. The plant-based crisp food product of claim 21 or 22, further comprising calcium carbonate.

25. The plant-based crisp food product of claim 21 or 22, wherein the pea protein is between about 15% and about 80% (w/w) of the plant-based crisp food product.

26. The plant-based crisp food product of claim 21 or 22, wherein the pea starch is between about 15% and about 90% (w/w) of the plant-based crisp food product.

27. The plant-based crisp food product of claim 26, further comprising a sugar.

28. The plant-based crisp food product of claim 27, wherein the sugar is between about 1% and about 5% of the plant-based crisp product.

29. The plant-based crisp food product of claim 21 or 22, further comprising calcium carbonate.

30. The plant-based crisp food product of claim 21 or 22, wherein the calcium carbonate comprises between about 0.1% and about 1% (w/w) of the plant-based crisp food product.

31. The plant-based crisp food product of claim 21 or 22, wherein the N-trans-caffeoyltyramine comprises about 0.01% to about 20% (w/w) of the plant-based crisp food product.

32. The plant-based crisp food product of claim 21 or 22, wherein the N-trans-feruloyltyramine comprises about 0.01% to about 20% (w/w) of the plant-based crisp food product.

33. The plant-based crisp food product of claim 21 or 22, wherein the food product contains less than about 1% THC (w/w) of the plant-based crisp food product.

34. The plant-based crisp food product of claim 21 or 22, wherein the food product comprises about 2% to about 10% (w/w) moisture plant-based crisp food product.

35. The plant-based crisp food product of claim 21 or 22, further comprising a fat source.

36. The plant-based crisp food product of claim 35, wherein the fat source comprises 5% to 20% (w/w) of the plant-based crisp food product.

37. The plant-based crisp food product of claim 21 or 22, wherein the food product comprises about 1% to about 10% (w/w) ash of the plant-based crisp food product.

38. The plant-based crisp food product of claim 21 or 22, wherein the food product has a carbohydrate to dietary fiber ratio from about 2:1 to about 1:2.

39. The plant-based crisp food product of claim 38, wherein the food product has a carbohydrate to dietary fiber ratio from about 1:1.

40. A process for preparing a food composition, comprising:
roasting hemp hulls to form roasted hemp hulls;
milling roasted hemp hulls to form milled roasted hemp hulls; and
feeding milled roasted hemp hull into an integrated extruder of form a food product of any one of claims 1 to 20.

41. The process of claim 40, further comprising pre-treating the milled roasted hemp hulls with water, enzymes, acid, base, or a combination there.

42. The process of claim 40 or 41, further comprising high shear mixing the milled roasted hemp hulls.

43. The process of any one of claims 40 to 41, further comprising puffing, pelletizing or shaping the milled roasted hemp hulls.

44. The process of any one of claims 40 to 43, further comprising vacuum drying the milled roasted hemp hulls.

45. The process of any one of claims 40 to 44, further comprising granulating the milled roasted hemp hulls.

46. The process of any one of claims 40 to 45, wherein roasting hemp hulls is at a temperature range from about 225° C. to about 275° C.

47. The process of any one of claims 40 to 46, wherein the milling roast hemp hulls is at speed from about 150 rpm to about 250 rpm.

48. A method for making a liquid product, the method comprising:
adding hemp hulls to an extruder at a speed from about 500 rpm to about 700 rpm, wherein the hemp hulls are added at a rate from about 3 lb/h to about 8 lb/h, wherein water is added from about 5 lb/h to about 10 lb/h, wherein the extruder temperature is from about 130° C. to about 150° C.; and
screening the hemp hulls with a mesh screen,
thereby collecting the liquid product.

49. A method for making an expanded food product, the method comprising:
forming a dough by admixing water and hemp hulls;
extrusion cooking the dough to form a cooked dough;
extruding the cooked dough through an extruder to form cooked dough pieces; and
cutting the cooked dough pieces,
wherein the cooked dough pieces retain one or more bioactives.

50. A method of producing a hemp hull containing composite food product, the method comprising:
extruding hemp hulls with an extruder, thereby forming a milled hemp hull; and
adding the milled hemp hull to addition food ingredients,
thereby producing a hemp hull containing composite food product.
